(19) United States
(12) Patent Application Publication
(10) Pub. No.: US 2023/0390224 A1
(43) Pub. Date: Dec. 7, 2023

(54) UNITARY ORAL DOSAGE FORM IN BASE OF SPHERICAL AND/OR SPHEROIDAL SHAPED PARTICLES

(71) Applicant: ATOMIC PHARMACEUTICS INC., Wilmington, DE (US)

(72) Inventors: Julián SECO CALVO, Las Condes (CL); Ignacio MURUA DAZA, Vitacura (CL)

(73) Assignee: ATOMIC PHARMACEUTICS INC., Wilmington, DE (US)

(21) Appl. No.: 18/205,308

(22) Filed: Jun. 2, 2023

Related U.S. Application Data
(60) Provisional application No. 63/348,688, filed on Jun. 3, 2022.

(51) Int. Cl. A61K 31/167; A61K 9/00; A61K 9/16; A61K 31/192; A61K 31/196; A61K 31/4402; A61K 31/165; A61K 31/235; A61K 31/00

(52) U.S. Cl. CPC A61K 31/167; A61K 9/0095; A61K 9/1652; A61K 9/1611; A61K 9/1664; A61K 9/1617; A61K 31/192; A61K 31/196; A61K 31/4402; A61K 31/165; A61K 31/235; A61K 31/658

(57) ABSTRACT

Described herein are therapeutic beverages that include a beverage type liquid medium and a plurality of spherical- and/or spheroidal-shaped particles dispersed within the liquid medium, wherein the plurality of the spherical- and/or spheroidal-shaped particles comprise at least one active pharmaceutical ingredient (API) disposed within the plurality of the spherical- and/or spheroidal-shaped particles. Also, described herein are methods of producing the described therapeutic beverages.

### RELATED APPLICATION

The present patent document claims the benefit of the filing date under 35 U.S.C. § 119(e) of Provisional U.S. Patent Application Ser. No. 63/348,688, filed Jun. 3, 2022, which is hereby incorporated by reference.

### BACKGROUND OF THE INVENTION

Described herein is a pharmaceutical dosage form, e.g., a single dose therapeutic formulation suitable for oral administration (e.g., in a form of a beverage) to a subject including spherical- and/or spheroidal-shaped particles suspended in a medium, e.g., an aqueous solution.

Nowadays, there are different pharmaceutical unit dosages for administration of active pharmaceutical ingredients (APIs) to obtain the desired therapeutic effect(s). At the same time, it is important to facilitate the drug administration so that patient compliance with treatment is achieved. (Pages-Puigdemont N., Valverde-Merino M., Medication Adherence; Modifiers and Improvement Strategies”, Ars Pharm; 59(4):251-258 (20-18)). It is well known that swallowing tablets and/or capsules may present a problem for a significant number of people. This can lead to a lack of compliance in treatments using such tablets or capsules. Also, often tablets or capsules have reduced stability, and undesired biocompatibility, inappropriate and/or deficient bioavailability and/or absorption rates may be generated when a pharmaceutical composition is administered.

For at least these reasons, there has been interest in formulations that include aqueous solutions. However, currently available formulations, although may allow for easier ingestion, have the disadvantage of not maintaining the stability of the APIs in said medium. For example, it is known that paracetamol in an aqueous solution can undergo hydrolysis to form p-aminophenol, which in turn can be degraded to quinoneimine. In this context, U.S. Pat. No. 8,404,891 discloses a storage-stable formulation of paracetamol in aqueous solution for use as an injectable solution, where a way of preparing said formulation is proposed, controlling different factors, such as; temperature, ambient (inert environment), pH which allows obtaining formulation stable for long periods of time. EP0859329 and US2004/0054012 describe processes for obtaining aqueous solutions that comprise paracetamol through a deoxygenation process that involves bubbling an inert gas, such as nitrogen through the aqueous solution. Therefore, in order to obtain stable solutions, it is necessary to carefully control the preparation processes, even raising production costs, in many cases.

In the pharmaceutical arts, different pharmaceutical forms have been studied and developed that include numerous pharmaceutical support(s) and/or coatings incorporating the APIs. Such pharmaceutical forms allow appropriate API administration, maintaining and/or controlling its release rate, as well as its stability in the system. In particular, different technologies have been developed over the years that solve problems with swallowing tablets and/or capsules. For example, alternatives to oral administration have been suggested, such as parenteral, transdermal, nasal, buccal, sublingual, or rectal administrations. Alternatively, easy-to-swallow oral dosage forms have also been developed, such as oral solutions, oral dispersible tablets, powders or granules to sprinkle on food, or oral gels. However, these solutions have many disadvantages.

Among drug delivery systems, the use of natural polymeric gels is potentially attractive. These polymers are used in the preparation of solid and semi-liquid oral pharmaceutical forms due to their binding and release-regulating properties. Said preparations are also attractive due to their physicochemical properties, since they have the capacity—under specific conditions—to form spherical- or spheroidal-shaped particles of different sizes. Furthermore, from an applied pharmaceutical perspective, gel compositions for oral administration are an attractive alternative to swallowing tablets and capsules.

Microparticles prepared on the basis of polymeric gels have played an important role in the progress of delivery systems, since they can encapsulate various drugs and small molecules, nucleic acids and proteins. Also, being biocompatible, they can offer superior bioavailability and are capable of being released over longer periods of time. (Abbas et al, Turk. J. Pharm. Sci., 2020; 17(2):159-171). In addition, the development in technology related to spherical microparticles, in the field of pharmaceutical administration has advanced with the processes application that include combinations of phase separations or precipitations, emulsion or solvent evaporation and spray methods, among others.

The drug delivery systems can be of two types: microcapsules and micromatrix. Microcapsules are reservoir systems where the drug is covered by a polymeric material. While, in micromatrix a drug can be uniformly dispersed in the polymeric matrix. (Nguyen et al., International Journal of Biological Macromolecules, 153:1035-1046 (2020)).

Among the most commonly used polymers for these purposes are polymers, such as alginate, chitosan, methyl cellulose, hydroxypropylmethyl cellulose, gellan gum and/or or mixtures of the same gelling agents, because they provide multiple physical, chemical and/or pharmacological advantages (Essa et al., Journal of Drug Delivery Science and Technology, 61 (2021)). Regarding gelling agents, gellan gum is a tetrasaccharide comprises of two D-glucose units: D-Glucuronic acid and L-rhamnose, which in aqueous solution ionizes, generating free carboxylate groups in its structure, which in the presence of divalent ions, complex two groups carboxylates by cation, forming the gel (Jahan, Nusrat, et al., “Gel point determination of gellan biopolymer gel from DC electrical conductivity” e-Polymers, 21(1):7-14 (2021)). Gellan gum does not present toxicity to living beings, nor does it have adverse effects on its intake, so it is recommended for use as a food additive. (EFSA Journal, 16(6):5296 (2018)). Gellan gum being useful for its ability to form gel, this tetrasaccharide has been widely used to “encapsulate” or trap drugs, in order to prevent adverse effects from direct administration of these drugs (T. Osmalek et al., “Gellan gum macrobeads loaded with naproxen: The impact of various naturally derived polymers on pH-dependent behavior,” Journal of Biomaterials Applications, 33(1):140-155 (2018)).

Similarly, another natural polymer with increases popularity is alginate: it is biodegradable, non-toxic, and biocompatible too, among other properties (Dewi Melani Hariyadi et al., Advances in Pharmacological and Pharmaceutical Sciences, Article ID 8886095, pg. 1-16 (2020)).

In general, three methods of preparing gels have been reported: spray drying, extrusion and emulsification/gelation proceeding. These last two are the most studied because the methods involve the formation of stable polymer droplets in their emulsification and gelation process, where the alginate is capable of forming gel in the presence of bivalent cations such as Ca²⁺, Ba²⁺ or Sr²⁺ (Nguyen Thi Thanh Uyen et al., International Journal of Biological Macromolecules, 153:1035-1046 (2020)).

Pharmaceutical forms are described in the literature that comprise polymer particles or microparticles, and it also contain different pharmaceutically active ingredients inside.

However, new pharmaceutical dosage forms that allows for an immediate and/or delayed release of APIs, without the disadvantages of the currently available pharmaceutical dosage forms, are desirable.

### SUMMARY

Described herein are stable pharmaceutical formulations in the form of e.g. a single dose therapeutic formulation (e.g., in a form of a beverage) that include spherical- or spheroidal-shaped particles with a therapeutically effective amount of APIs which are at the same time suspended in a liquid that can be immediately and easily ingested. The described formulations, surprisingly, provide a distribution of the API in the particles and in the liquid that comprises them, facilitate the administration in comparison with other commercially available formulations, for example, capsules and/or tablets, and at the same time ensure a delivery of the API as prescribed.

One embodiment relates to a single dose therapeutic beverage, comprising: a beverage type liquid medium; and a plurality of spherical- and/or spheroidal-shaped particles dispersed within the liquid medium; wherein the plurality of the spherical- and/or spheroidal-shaped particles comprise at least one active pharmaceutical ingredient (API) disposed within the plurality of the spherical- and/or spheroidal-shaped particles; wherein the API partially permeates into the beverage type liquid medium from within the plurality of the spherical- and/or spheroidal-shaped particles so that an amount of the API is partitioned between the plurality of the spherical- and/or spheroidal-shaped particles and the liquid medium containing the particles; and wherein, upon administration to a subject, the therapeutic beverage is configured to provide an immediate dose of the API and a subsequent delayed release dose of the API. The percentage of APIs present in the liquid medium is from about 10% to about 50% w/v and the percentage of APIs present in the plurality of the spherical- and/or spheroidal-shaped particles is from about 50% to about 90% w/w; or the percentage of APIs present in the liquid medium is from about is from about 15% to about 35% w/v and the percentage of APIs present in the plurality of the spherical- and/or spheroidal-shaped particles is from about 65% to about 85% w/w; or the percentage of APIs present in the liquid medium is about 30% w/v and the percentage of APIs present in the plurality of the spherical- and/or spheroidal-shaped particles is about 70% w/w. The API may be selected from the group consisting of analgesics, nonsteroidal anti-inflammatory drugs (NSAIDs), Cannabis derivatives, antiallergics expectorants, antitussives, antibiotics, diuretics, hypotensives, hypoglycemics, antispasmodics, antidepressants, anti-ulcer drugs, vitamins, and supplements. The plurality of the spherical- and/or spheroidal-shaped particles may further comprises at least one of: one or more gelling agents selected from the group consisting of gellan gum, methyl cellulose, methyl ethyl cellulose, hydroxy propyl methyl cellulose, locust bean gum, carrageenan, sodium alginate, xanthan gum, gelatine, chitosan, derivatives of chitosan, and/or mixtures thereof; and one or more agents for spherification selected from the group consisting of calcium chloride, calcium acetate, calcium citrate, calcium phosphate, monobasic calcium phosphate, and calcium gluconolactate. The single dose therapeutic beverage may further comprise one or more emulsifying agents selected from the group consisting of lecithin, fatty acid diglycerides and fatty acid esters, and polysaccharides. The single dose therapeutic beverage may further comprise one or more oily dispersing agents selected from the group consisting of canola oil, olive oil, almond oil, chia oil, corn oil, marigold oil, coconut oil, soybean oil, mineral oils and the like. The single dose therapeutic beverage may further comprise excipients selected from the group consisting of stabilizers, preservatives, antioxidants, viscosifying agents, flavoring agents, sweeteners, colorings, and pH stabilizers. The plurality of the spherical- and/or spheroidal-shaped particles may have a diameter in a range from about 100 μm to about 6000 μm, from about 1000 μm to about 3000 μm, or from about 2000 μm to about 3000 μm. The therapeutic beverage may be a packaged beverage provided in a unit that contains between about 10-1000 mL of the beverage, or between about 10-500 mL of the therapeutic beverage. The therapeutic beverage may be in a form of an instant beverage. The beverage may be for daily consumption once or more times a day. In the single dose therapeutic beverage, the API may comprise a first API and a second API, different from the first API, and the first API is disposed within a first set of the plurality of spherical- and/or spheroidal-shaped particles and the second API that may be disposed with a second set of the plurality of spherical- and/or spheroidal-shaped particles.

Another embodiment relates to a therapeutic beverage, comprising: a beverage type liquid medium; and a plurality of spherical- and/or spheroidal-shaped particles dispersed within the liquid medium, wherein the plurality of the spherical- and/or spheroidal-shaped particles comprise at least one active pharmaceutical ingredient (API) disposed within the plurality of the spherical- and/or spheroidal-shaped particles; wherein the spherical- and/or spheroidal-shaped particles have a diameter in a range from about 100 μm to about 6000 μm; and wherein the API partially permeates into the beverage like liquid medium from within the plurality of the spherical- and/or spheroidal-shaped particles so that an amount of the API is partitioned between the plurality of the spherical- and/or spheroidal-shaped particles and the beverage like liquid medium containing the particles; and wherein, upon administration to a subject, the therapeutic beverage is configured to provide an immediate dose of the API and a subsequent delayed release dose of the API. The therapeutic beverage may be a single dose therapeutic beverage disposed in a beverage container. The percentage of APIs present in the liquid medium may be from about 10% to about 50% w/v and the percentage of APIs present in the plurality of the spherical- and/or spheroidal-shaped particles is from about 50% to about 90% w/w; or the percentage of APIs present in the liquid medium may be from about is from about 15% to about 35% w/v and the percentage of APIs present in the plurality of the spherical- and/or spheroidal-shaped particles is from about 65% to about 85% w/w; or the percentage of APIs present in the liquid medium may be about 30% w/v and the percentage of APIs present in the spherical- and/or spheroidal-shaped particles is about 70% w/w. The API may comprise a first API and a second API, different from the first API, and the first API may be disposed within a first set of the plurality of spherical- and/or spheroidal-shaped particles and the second API may be disposed with a second set of the plurality of spherical- and/or spheroidal-shaped particles. The API may be selected from the group consisting of analgesics, nonsteroidal anti-inflammatory drugs (NSAIDs), Cannabis derivatives, antiallergics expectorants, antitussives, antibiotics, diuretics, hypotensives, hypoglycemics, antispasmodics, antidepressants, anti-ulcer drugs, vitamins, and supplements. The therapeutic beverage may further comprise excipients selected from the group consisting of stabilizers, preservatives, antioxidants, viscosifying agents, flavoring agents, sweeteners, colorings, and pH stabilizers.

Yet another embodiment relates to a single dose therapeutic beverage, comprising: a beverage container; a beverage type liquid medium disposed within the container; and a plurality of spherical- and/or spheroidal-shaped particles dispersed within the liquid medium; wherein the plurality of the spherical- and/or spheroidal-shaped particles comprise at least one pharmaceutically active ingredient (API) disposed within the plurality of the spherical- and/or spheroidal-shaped particles; wherein the spherical- and/or spheroidal-shaped particles have a diameter in a range from about 1000 μm to about 3000 μm; wherein the beverage container contains between about 10-1000 mL of the single dose therapeutic beverage or between about 10-500 mL of the therapeutic beverage; wherein the API partially permeates into the beverage like liquid medium from within the spherical- and/or spheroidal-shaped particles so that an amount of the API is partitioned between the spherical- and/or spheroidal-shaped particles and the beverage like liquid medium containing the particles; and wherein, upon administration to a subject, the therapeutic beverage is configured to provide an immediate dose of the API and a subsequent delayed release dose of the API. The API may comprise a first API and a second API, different from the first API, and the first API may be disposed within a first set of the plurality of spherical- and/or spheroidal-shaped particles and the second API may be disposed with a second set of the plurality of spherical- and/or spheroidal-shaped particles. The percentage of APIs present in the liquid medium may be from about 10% to about 50% w/v and the percentage of APIs present in the plurality of the spherical- and/or spheroidal-shaped particles is from about 50% to about 90% w/w; or the percentage of APIs present in the liquid medium may be from about is from about 15% to about 35% w/v and the percentage of APIs present in the plurality of the spherical- and/or spheroidal-shaped particles is from about 65% to about 85% w/w; or the percentage of APIs present in the liquid medium may be about 30% w/v and the percentage of APIs present in the spherical- and/or spheroidal-shaped particles is about 70% w/w. The API may be selected from the group consisting of analgesics, nonsteroidal anti-inflammatory drugs (NSAIDs), Cannabis derivatives, antiallergics expectorants, antitussives, antibiotics, diuretics, hypotensives, hypoglycemics, antispasmodics, antidepressants, anti-ulcer drugs, vitamins, and supplements. The therapeutic beverage may further comprise excipients selected from the group consisting of stabilizers, preservatives, antioxidants, viscosifying agents, flavoring agents, sweeteners, colorings, and pH stabilizers.

Yet another embodiment relates to a single dose therapeutic beverage, comprising: a beverage container; a beverage type liquid medium disposed within the container; and a plurality of spherical- and/or spheroidal-shaped particles dispersed within the liquid medium; wherein the spherical- and/or spheroidal-shaped particles comprise at least one pharmaceutically active ingredient (API) disposed within the spherical- and/or spheroidal-shaped particles; wherein the spherical- and/or spheroidal-shaped particles have a diameter in a range from about 100 μm to about 6000 μm; wherein the beverage container contains between 10-1000 mL of the therapeutic beverage; wherein the API partially permeates into the beverage like liquid medium from within the spherical- and/or spheroidal-shaped particles so that an amount of the API is partitioned between the spherical- and/or spheroidal-shaped particles and the beverage like liquid medium containing the particles; wherein, upon administration to a subject, the therapeutic beverage is configured to provide an immediate dose of the API and a subsequent delayed release dose of the API; and wherein the percentage of APIs present in the liquid medium is from about 10% to about 50% w/v and the percentage of APIs present in the plurality of the spherical- and/or spheroidal-shaped particles is from about 50% to about 90% w/w. The API may comprise a first API and a second API, different from the first API, and the first API may be disposed within a first set of the plurality of spherical- and/or spheroidal-shaped particles and the second API may be disposed with a second set of the plurality of spherical- and/or spheroidal-shaped particles. The percentage of APIs present in the liquid medium may be about 30% w/v and the percentage of APIs present in the spherical- and/or spheroidal-shaped particles is about 70% w/w. The API may be selected from the group consisting of analgesics, nonsteroidal anti-inflammatory drugs (NSAIDs), Cannabis derivatives antiallergics expectorants, antitussives, antibiotics, diuretics, hypotensives, hypoglycemics, antispasmodics, antidepressants, anti-ulcer drugs, vitamins, and supplements.

Yet further embodiment relates to a single dose therapeutic beverage, comprising: a beverage container; a beverage type liquid medium disposed within the container; and a plurality of spherical- and/or spheroidal-shaped particles dispersed within the liquid medium; wherein the plurality of the spherical- and/or spheroidal-shaped particles comprise at least one active pharmaceutical ingredient (API) disposed within the plurality of the spherical- and/or spheroidal-shaped particles; wherein the spherical- and/or spheroidal-shaped particles have a diameter in a range from about 100 μm to about 6000 μm; wherein the beverage container contains between about 10-1000 mL of the single dose therapeutic beverage, or between about 10-500 mL of the single dose therapeutic beverage; and wherein the percentage of APIs present in the liquid medium is from about 10% to about 50% w/v and the percentage of APIs present in the plurality of the spherical- and/or spheroidal-shaped particles is from about 50% to about 90% w/w; or the percentage of APIs present in the liquid medium is from about is from about 15% to about 35% w/v and the percentage of APIs present in the plurality of the spherical- and/or spheroidal-shaped particles is from about 65% to about 85% w/w; or the percentage of APIs present in the liquid medium is about 30% w/v and the percentage of APIs present in the plurality of the spherical- and/or spheroidal-shaped particles is about 70% w/w. The plurality of the spherical- and/or spheroidal-shaped particles can have a diameter in a range from about 100 μm to about 4000 μm, from about 1000 μm to about 3000 μm, or from about 2000 μm to about 3000 μm. The API may be selected from the group consisting of analgesics, nonsteroidal anti-inflammatory drugs (NSAIDs), Cannabis derivatives, antiallergics, expectorants, antitussives, antibiotics, diuretics, hypotensives, hypoglycemics, antispasmodics, antidepressants, anti-ulcer drugs, vitamins, and supplements. The plurality of the spherical- and/or spheroidal-shaped particles may further comprise at least one of: one or more gelling agents selected from the group consisting of gellan gum, methyl cellulose, methyl ethyl cellulose, hydroxy propyl methyl cellulose, locust bean gum, carrageenan, sodium alginate, xanthan gum, gelatine, chitosan, derivatives of chitosan, and/or mixtures thereof; and one or more agents for spherification selected from the group consisting of calcium chloride, calcium acetate, calcium citrate, calcium phosphate, monobasic calcium phosphate, and calcium gluconolactate. The single dose therapeutic beverage may further comprise one or more emulsifying agents selected from the group consisting of lecithin, fatty acid diglycerides and fatty acid esters, and polysaccharides. The single dose therapeutic beverage may further comprise one or more oily dispersing agents selected from the group consisting of canola oil, olive oil, almond oil, chia oil, corn oil, marigold oil, coconut oil, soybean oil, mineral oils and the like. The single dose therapeutic beverage may further comprise excipients selected from the group consisting of stabilizers, preservatives, antioxidants, viscosifying agents, flavoring agents, sweeteners, colorings, and pH stabilizers. The therapeutic beverage may be in a form of an instant beverage. The beverage may be for daily consumption once or more times a day. In the therapeutic beverage, the API comprises a first API and a second API, different from the first API, and the first API is disposed within a first set of the plurality of spherical- and/or spheroidal-shaped particles and the second API is disposed with a second set of the plurality of spherical- and/or spheroidal-shaped particles.

Yet another embodiment relates to a method of preparing a single dose therapeutic beverage, comprising: i) suspending at least one active pharmaceutical ingredient (API) in an aqueous medium and/or in an oily medium; ii) adding at least one gelling agent to step i) while stirring to produce a mixture; iii) dropping the mixture obtained in step ii) onto a spherification agent while stirring to produce spherical- and/or spheroidal-shaped particles comprising the at least one API; iv) removing by filtration the spherification agent to separate spherical- and/or spheroidal-shaped particles comprising the at least one API; v) washing the separated spherical- and/or spheroidal-shaped particles comprising the at least one API with demineralized and/or deionized water; and vi) suspending the washed spherical- and/or spheroidal-shaped particles comprising the at least one API in the liquid medium; and vii. introducing the single dose therapeutic beverage into a beverage container; wherein the API partially permeates into the beverage like liquid medium from within the spherical- and/or spheroidal-shaped particles so that an amount of the API is partitioned between the spherical- and/or spheroidal-shaped particles and the beverage like liquid medium containing the particles; wherein, upon administration to a subject, the therapeutic beverage is configured to provide an immediate dose of the API and a subsequent delayed release dose of the API; and wherein the percentage of APIs present in the liquid medium is from about 10% to about 50% w/v and the percentage of APIs present in the plurality of the spherical- and/or spheroidal-shaped particles is from about 50% to about 90% w/w. In the method, in step iii) the spherification agent can contain at least one gelling agent. In the method, in step ii) the temperature may be from about 25° C. to about 60° C. In the method, in step iii) the spherification agent may be CaCl₂ solution in the range from about 1.5% to about 6% w/v.

Yet another embodiment relates to a method of preparing a single dose therapeutic beverage, comprising: i) suspending at least one active pharmaceutical ingredient (API) in an alkaline medium and/or in an oily medium; ii) adding a gelling agent to step i) while stirring to produce a mixture; iii) dropping the mixture obtained in step ii) onto another gelling agent while stirring to produce spherical- and/or spheroidal-shaped particles comprising the at least one API; iv) removing by filtration the spherification agent to separate spherical- and/or spheroidal-shaped particles comprising the at least one API; v) washing the separated spherical- and/or spheroidal-shaped particles comprising the at least one API with demineralized and/or deionized water; and vi) suspending the washed spherical- and/or spheroidal-shaped particles comprising the at least one API in the liquid medium; and vii. introducing the single dose therapeutic beverage into a beverage container; wherein the API partially permeates into the beverage like liquid medium from within the spherical- and/or spheroidal-shaped particles so that an amount of the API is partitioned between the spherical- and/or spheroidal-shaped particles and the beverage like liquid medium containing the particles; wherein, upon administration to a subject, the therapeutic beverage is configured to provide an immediate dose of the API and a subsequent delayed release dose of the API; and wherein the percentage of APIs present in the liquid medium is from about 10% to about 50% w/v and the percentage of APIs present in the plurality of the spherical- and/or spheroidal-shaped particles is from about 50% to about 90% w/w. In the method, in step ii) the temperature may be from about 25° C. to about 60° C. In the method, in step iii) the spherification agent may be CaCl₂ solution in the range from about 1.5% to about 6% w/v.

### BRIEF DESCRIPTION OF THE DRAWINGS

The described embodiments are illustrated but not limited by the accompanying drawings, in which:

FIGS. 1a), 1b) and 1c) show spherical particles of chloramphenicol obtained at 25° C., by gelation in a mixture of alginate/water (80:20% w/v) and 20 mM CaCl₂ solution: FIG. 1a) shows spherical wet chloramphenicol particles; FIG. 1b) shows aqueous solution with spherical chloramphenicol particles suspended, front view; FIG. 1c) shows aqueous solution with spherical particles of chloramphenicol suspended, top view.

FIGS. 2a), 2b) and 2c show spherical particles of chloramphenicol obtained at 25° C., by gelation in a mixture of alginate/canola oil (80:20% w/v) and 20 mM CaCl₂ solution: FIG. 2a) shows spherical wet chloramphenicol particles; FIG. 2b) shows spherical particles of chloramphenicol in aqueous solution, front view; and FIG. 2c) shows spherical particles of chloramphenicol in aqueous solution, top view.

FIGS. 3a), 3b) and 3c) show spherical particles of chloramphenicol obtained at 25° C., by gelation with a mixture of alginate/water (80:20% w/v, chitosan solution 1.0% w/v) and 20 mM CaCl₂ solution: FIG. 3a) shows spherical wet chloramphenicol particles; FIG. 3b) shows spherical particles of chloramphenicol in aqueous solution, front view; and FIG. 3c) shows spherical particles of chloramphenicol in aqueous solution, top view.

FIGS. 4a)-4f) show Examples of Microscopic images (SEM) of spherical particles of acetaminophen obtained at different conditions: FIG. 4a) shows spherical dry-wet acetaminophen particles: alginate/water (80:20% w/v, chitosan solution 1.0% w/v) and 20 mM CaCl₂ solution, using a 10 mL syringe with an 18-gauge needle; FIG. 4b) shows spherical dry-wet acetaminophen particles: alginate/oil (80:20% w/v, chitosan solution 1.0% w/v) and 20 mM CaCl₂ solution, using a 10 mL syringe with an 18-gauge needle; FIG. 4c) shows spherical dry-wet chloramphenicol particles: alginate/water (80:20% w/v, chitosan solution 1.0% w/v) and 20 mM CaCl₂ solution, using a 10 mL syringe with an 18-gauge needle; FIG. 4d) shows spherical dry-wet chloramphenicol particles: alginate/oil (80:20% w/v, chitosan solution 1.0% w/v) and 20 mM CaCl₂ solution, using a 10 mL syringe with an 18-gauge needle; FIG. 4e) shows spherical dry-wet acetaminophen particles: alginate/oil (80:20% w/v, chitosan solution 1.0% w/v) and 20 mM CaCl₂ solution, using a 10 mL syringe with a 21-gauge needle; FIG. 4f) shows spherical dry-wet chloramphenicol particles: alginate/oil (80:20% w/v, chitosan solution 1.0% w/v) and 20 mM CaCl₂ solution, using a 10 mL syringe with a 21-gauge needle; FIG. 4g) shows spherical dry-wet acetaminophen particles: alginate/oil (80:20% w/v, chitosan solution 1.0% w/v) and 20 mM CaCl₂ solution, using a 10 mL syringe with a 27-gauge needle; and FIG. 4h) shows spherical dry-wet acetaminophen particles: alginate/oil (80:20% w/v, chitosan solution 1.0% w/v) and 20 mM CaCl₂ solution, using a 10 mL syringe with a 10-gauge needle.

FIG. 5 shows release curves of acetaminophen in liquid medium B (CaCl₂ 3% w/v), for combinations of gelling agents (Alg, alginate; Ch, chitosan) with 21-gauge needle.

FIG. 6 shows release curves of acetaminophen in liquid medium B (CaCl₂ 3% w/v), for combinations of gelling agents (Alg, alginate; Ch, chitosan) with 18-gauge needle.

FIG. 7 shows release curves of acetaminophen in liquid medium B (3 and 6% w/v of CaCl₂), for gellan gum (GG) gelling agent with 18-gauge needle.

FIG. 8 shows release curves of acetaminophen in liquid medium C with 20 mM sodium citrate, for combinations of gelling agents (Alg, alginate; Ch, chitosan) with 27-gauge needle.

FIG. 9 shows release curves of acetaminophen in liquid medium C with 20 mM sodium citrate, for combinations of gelling agents (Alg, alginate; Ch, chitosan) with 18-gauge needle.

FIG. 10 shows release curves of acetaminophen in liquid medium C with 20 mM sodium citrate, for combinations of gelling agents (Alg, alginate; Ch, chitosan) with 10-gauge needle.

FIG. 11 shows release curves of acetaminophen in liquid medium B (CaCl₂ 3% w/v), for combinations of gelling agents (Alg, alginate; Ch, chitosan; Pec, pectin) with 18-gauge needle.

FIG. 12 shows release curves of acetaminophen in liquid medium B (CaCl₂ 3% w/v), for combinations of gelling agents (Alg, alginate; Ch, chitosan; Starch) with 18-gauge needle.

FIG. 13 shows release curves of acetaminophen in liquid medium C with 20 mM sodium citrate, for combinations of gelling agents (Alg, alginate; Ch, chitosan, Starch) with 18-gauge needle.

FIG. 14 shows release curves of acetaminophen in liquid medium B (CaCl₂ 3% w/v), for carrageenan (Carr) gelling agent with 18-gauge needle.

FIG. 15 shows release curves of acetaminophen in liquid medium C with 20 mM sodium citrate, for carrageenan (Carr) gelling agent with 18-gauge needle.

FIG. 16 shows release curves of naproxen in liquid medium B (CaCl₂ 3% w/v), for combinations of gelling agents (Alg, alginate; Ch, chitosan) with 18-gauge needle.

FIG. 17 shows release curves of naproxen in liquid medium B (3 and 6% w/v of CaCl₂), for gellan gum (GG) gelling agent with 18-gauge needle.

FIG. 18 shows release curves of naproxen in liquid medium C with 20 mM sodium citrate, for combinations of gelling agents (Alg, alginate; Ch, chitosan) with 18-gauge needle.

FIG. 19 shows release curves of chlorphenamine maleate (CM) in liquid medium B (CaCl₂ 3% w/v), for combinations of gelling agents (Alg, alginate; Ch, chitosan) with 18-gauge needle.

FIG. 20 shows release curves of chlorphenamine maleate (CM) in liquid medium C with 20 mM sodium citrate, for combinations of gelling agents (Alg, alginate; Ch, chitosan) with 18-gauge needle.

FIG. 21 shows release curves of chlorphenamine maleate (CM) in liquid medium B (3% w/v of CaCl₂), for combinations of gelling agent (GG, gellan gum; Ch, chitosan) with 18-gauge needle.

FIG. 22 shows release curves of chlorphenamine maleate (CM) in liquid medium C with 20 mM sodium citrate, for combinations of gelling agents (GG, gellan gum; Ch, chitosan) with 18-gauge needle.

FIG. 23 shows release curves of chloramphenicol in liquid medium B (CaCl₂ 3% w/v), for combinations of gelling agents (Alg, alginate; Ch, chitosan) with 18-gauge needle.

FIG. 24 shows release curves of chloramphenicol in liquid medium C with 20 mM sodium citrate, for combinations of gelling agents (Alg, alginate; Ch, chitosan) with 18-gauge needle.

FIG. 25 shows release curves of acetyl salicylic acid (ASA) in liquid medium B (CaCl₂ 3% w/v), for combinations of gelling agents (Alg, alginate; Ch, chitosan) with 18-gauge needle.

FIG. 26 shows release curves of acetyl salicylic acid (ASA) in liquid medium C with 20 mM sodium citrate, for combinations of gelling agents (Alg, alginate; Ch, chitosan) with 18-gauge needle.

FIG. 27 shows release curves of acetyl salicylic acid (ASA) in liquid medium B (3% w/v of CaCl₂), for gellan gum (GG) gelling agent with 18-gauge needle.

FIG. 28 shows release curves of acetyl salicylic acid (ASA) in liquid medium C with 20 mM sodium citrate, for combinations of gelling agents (GG, gellan gum; Ch, chitosan) with 18-gauge needle.

FIG. 29 shows release curves of acetyl salicylic acid (ASA) in liquid medium C with 20 mM sodium citrate, for combinations of gelling agents (Alg, alginate; Ch, chitosan) with 27-gauge needle.

FIG. 30 shows release curves of ibuprofen in liquid medium B (3% w/v of CaCl₂), for alginate (Alg) gelling agent with 18-gauge needle.

FIG. 31 shows release curves of ibuprofen in liquid medium C with 20 mM sodium citrate, for alginate (Alg) gelling agent with 18-gauge needle.

FIG. 32 shows release curves of acetaminophen. Formulation composed by acetaminophen (A) and acetyl salicylic acid (ASA), in liquid medium C with 20 mM sodium citrate, for combinations of gelling agents (Alg, alginate; Ch, chitosan) with 18-gauge needle.

FIG. 33 shows release curves of acetyl salicylic acid. Formulation composed by acetaminophen (A) and acetyl salicylic acid (ASA), in liquid medium C with 20 mM sodium citrate, for combinations of gelling agents (Alg, alginate; Ch, chitosan) with 18-gauge needle.

FIG. 34 shows release curves of acetaminophen (A) and acetyl salicylic acid (ASA) in a formulation, in liquid medium C with 20 mM sodium citrate, for combinations of gelling agents (Alg, alginate; Ch, chitosan) with 18-gauge needle.

FIG. 35 shows dissolution tests for pharmaceutical formulation that include acetaminophen (A) and excipients, in liquid medium C with 20 mM sodium citrate, for alginate (Alg) gelling agent with 18-gauge needle.

FIG. 36 shows dissolution tests for acetaminophen from spheres of pharmaceutical formulations A and B. Simulated intestinal fluid (pH 6.8).

FIG. 37 shows dissolution tests for acetaminophen from spheres and liquid medium C (20 mM sodium citrate) of pharmaceutical formulations A and B. Simulated intestinal fluid (pH 6.8).

FIG. 38 shows release fraction (%) for acetaminophen from spheres and liquid medium C (20 mM sodium citrate) of pharmaceutical formulations A and B. Simulated intestinal fluid (pH 6.8).

FIG. 39 shows dissolution tests for acetaminophen from spheres of pharmaceutical formulations A and B. Simulated duodenum fluid (pH 5.6).

FIG. 40 shows dissolution tests for acetaminophen from spheres and liquid medium C (20 mM sodium citrate) of pharmaceutical formulations A and B. Simulated duodenum fluid (pH 5.6).

FIG. 41 shows release fraction (%) for acetaminophen from spheres and liquid medium C (20 mM sodium citrate) of pharmaceutical formulations A and B. Simulated duodenum fluid (pH 5.6).

FIG. 42 shows dissolution tests for acetaminophen from spheres of pharmaceutical formulations A and B. Simulated stomach gastric fluid (pH 1.2).

FIG. 43 shows dissolution tests for acetaminophen from spheres and liquid medium C (20 mM sodium citrate) of pharmaceutical formulations A and B. Simulated stomach gastric fluid (pH 1.2).

FIG. 44 shows release fraction (%) for acetaminophen from spheres and liquid medium C (20 mM sodium citrate) of pharmaceutical formulations A and B. Simulated stomach gastric fluid (pH 1.2).

FIG. 45 shows comparison of acetaminophen release fraction (%) of pharmaceutical formulation A at different pH's.

FIG. 46 shows comparison of acetaminophen release fraction (%) of pharmaceutical formulation B at different pH's.

FIG. 47 shows maximum release (mg) at 10 min, acetaminophen in liquid medium B (3% w/v CaCl₂), for combinations of gelling agents (Alg, alginate; Ch, chitosan) with 27-gauge needle.

FIG. 48 shows maximum release (mg) at 10 min, acetaminophen in liquid medium B (3% w/v CaCl₂), for combinations of gelling agents (Alg, alginate; Ch, chitosan) with 18-gauge needle.

FIG. 49 shows maximum release (mg) at 10 min, acetaminophen in liquid medium B (3% w/v CaCl₂), for combinations of gelling agents (Alg, alginate; Ch, chitosan) with 10-gauge needle.

FIG. 50 shows maximum release (mg) at 10 min, acetaminophen in liquid medium B (3% w/v CaCl₂), for combinations of gelling agents (Alg, alginate; Starch; Ch, chitosan) with 18-gauge needle.

FIG. 51 shows maximum release (mg) at 10 min, acetaminophen in liquid medium B (3% w/v CaCl₂), for combinations of gelling agents (Pec, Pectin; Alg, Alginate; Ch, chitosan) with 18-gauge needle.

FIG. 52 shows maximum release (mg) at 10 min, acetaminophen in liquid medium B (3% w/v CaCl₂), for carrageenan gelling agent with 18-gauge needle.

FIG. 53 shows maximum release (mg) at 10 min, naproxen in liquid medium B (3% w/v CaCl₂), for combinations of gelling agents (Alg, alginate; Ch, chitosan) with 18-gauge needle.

FIG. 54 shows maximum release (mg) at 10 min, naproxen in liquid medium B (3 and 6% w/v of CaCl₂), for gellan gum (GG) gelling agent with 18-gauge needle.

FIG. 55 shows maximum release (mg) at 10 min, chlorphenamine maleate in liquid medium B (3% w/v CaCl₂), for combinations of gelling agents (Alg, alginate; Ch, chitosan) with 18-gauge needle.

FIG. 56 shows maximum release (mg) at 10 min, chlorphenamine maleate in liquid medium B (3% w/v CaCl₂), for combinations of gelling agents (GG, gellan gum; Ch, chitosan) with 18-gauge needle.

FIG. 57 shows maximum release (mg) at 10 min, chloramphenicol in liquid medium B (3% w/v CaCl₂), for combinations of gelling agents (Alg, alginate; Ch, chitosan) with 18-gauge needle.

FIG. 58 shows maximum release (mg) at 10 min, acetyl salicylic acid in liquid medium B (3% w/v CaCl₂), for combinations of gelling agents (Alg, alginate; Ch, chitosan) with 18-gauge needle.

FIG. 59 shows maximum release (mg) at 10 min, acetyl salicylic acid in liquid medium B (3% w/v CaCl₂), for combinations of gelling agents (GG, gellan gum; Ch, chitosan) with 18-gauge needle.

FIG. 60 shows maximum release (mg) at 10 min, acetyl salicylic acid in liquid medium B (3% w/v CaCl₂), for combinations of gelling agents (Alg, alginate; Ch, chitosan) with 27-gauge needle.

FIG. 61 shows the Scheme 1, General Experimental Procedure Using Ionotropic Gelation.

FIG. 62 shows Scheme 2, General Experimental Procedure Using Ionotropic Gelation By Formation Of Polyelectrolyte Complexes.

FIG. 63 shows Scheme 3. General Experimental Procedure Using Reverse Gelation by Formation Of Polyelectrolyte Complexes.

FIG. 64 shows a graph showing the relationship between the number of particles and mg of API (acetaminophen).

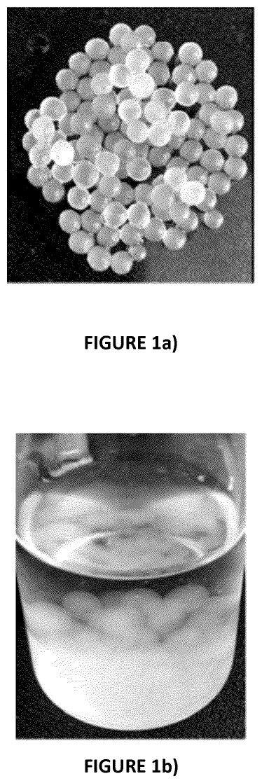

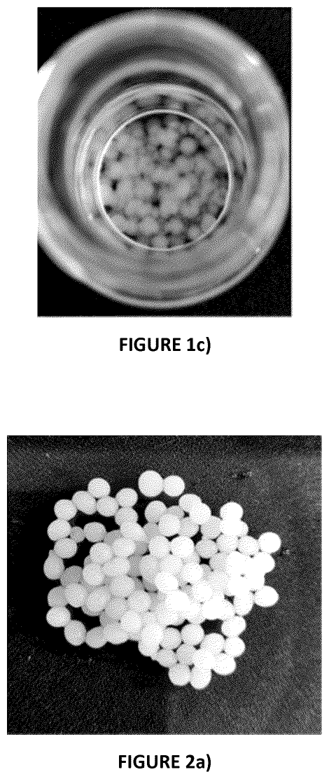

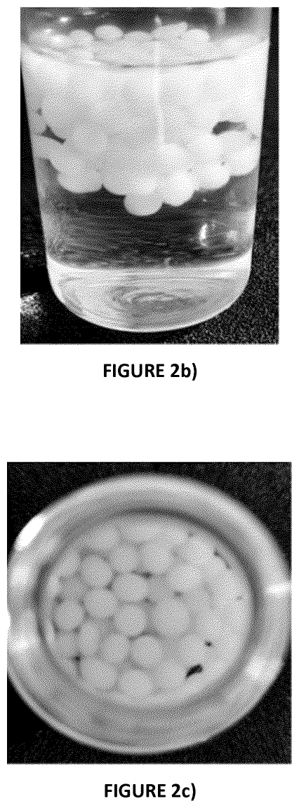

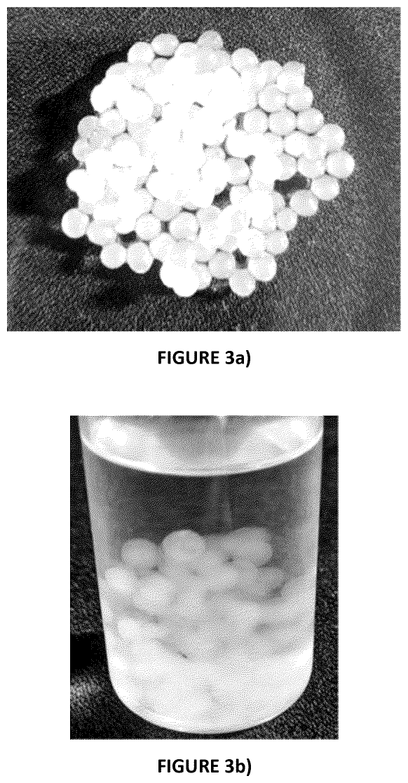

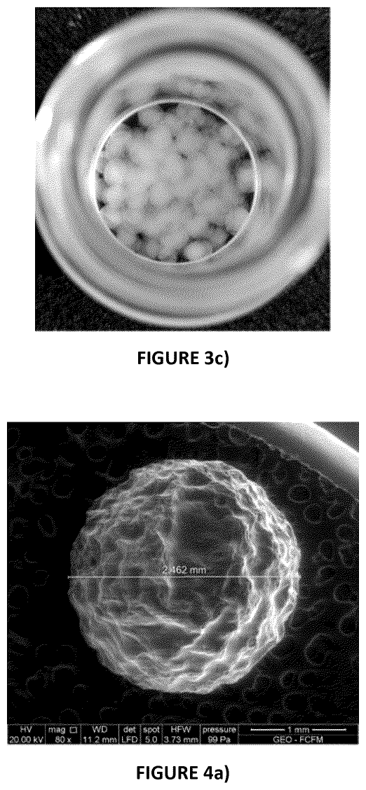

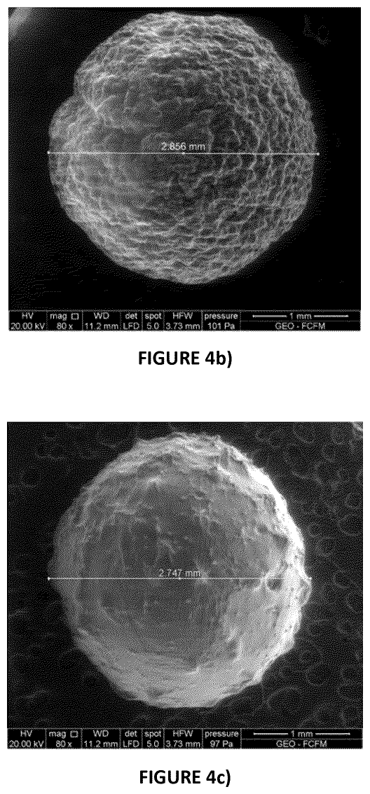

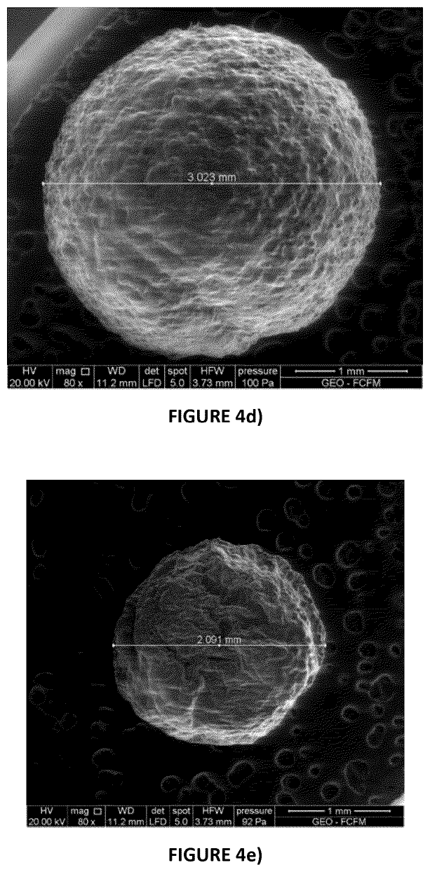

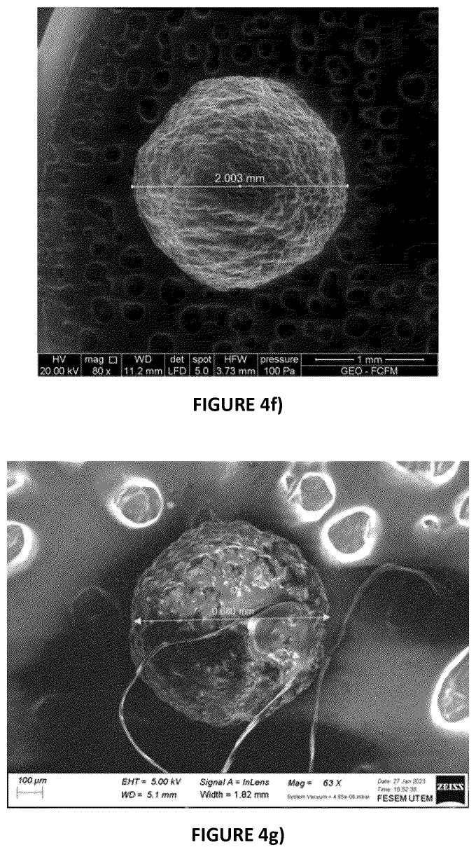

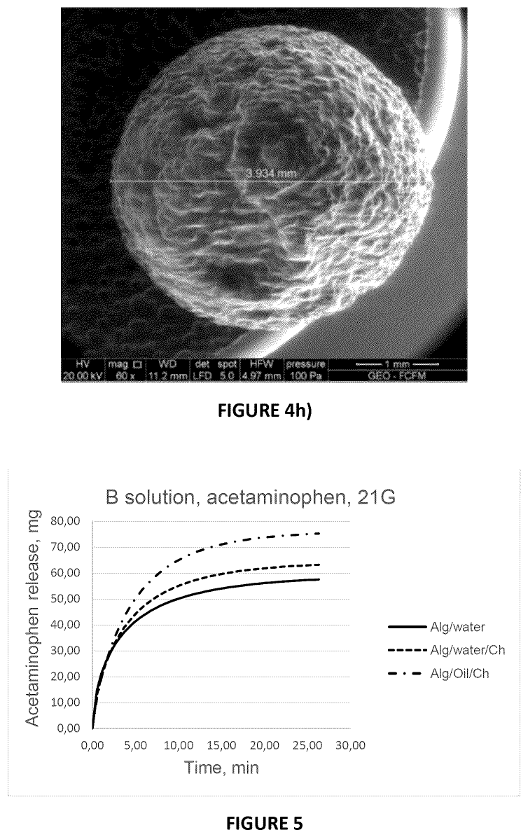

### DETAILED DESCRIPTION

It was surprisingly discovered that it is possible to produce a stable oral pharmaceutical formulation in the form of, e.g., a single dose therapeutic formulation (e.g., a beverage) including gelled spherical- or spheroidal-shaped particles that comprise one or more active pharmaceutical ingredient(s) (API(s)) in an amount sufficient to have a desired effect (“effective dose”) upon administration to a subject, dissolved and/or dispersed in a liquid, dispersed in an oil or in oil form, or by forming an emulsion within the spherical- or spheroidal-shaped particles, and where said spherical- or spheroidal-shaped particles are suspended in an aqueous liquid suitable for immediate ingestion by a subject.

The API is mixed with water or oil or an emulsion and mixed with the polymer to create the spherical- or spheroidal-shaped particles. After the spherical- or spheroidal-shaped particles are made, the particles are dispersed in a liquid that can be water or a beverage and contained it in a container (can, bottle, tetrapack, etc.) to create the final product. The spherical- or spheroidal-shaped particles solubilize some of the API into the liquid until an equilibrium of API in the liquid and API in the spheres is created. This allows to create a new beverage containing medicine (i.e., API) with a pleasant taste and that partitions part of the API inside the spherical- or spheroidal-shaped particles and part of the API in the liquid, interacting in an equilibrium between the API in the particles and the liquid.

It was observed that each API reacts different between the liquid, polymer, emulsion and excipients. When one variable is changed, a new equilibrium in the pharmaceutical form will be formed.

In one embodiment, described herein is a pharmaceutical dosage form, e.g., a single dose therapeutic formulation suitable for oral administration (e.g., in a form of a beverage) to a subject including spherical- and/or spheroidal-shaped particles suspended in a medium, e.g., an aqueous solution. In the single dose therapeutic formulation, the spherical- and/or spheroidal-shaped particles include at least one active pharmaceutical ingredient (API). The API can be dissolved and/or dispersed in a liquid phase, dispersed in an oil phase or as oil API, or in emulsion, inside the spherical- and/or spheroidal-shaped particles. The single dose therapeutic formulation is designed for human consumption, especially for an easy intake. Also, described herein is a method of preparing the single dose therapeutic formulation as well as its use in treatment of various diseases or disorders, which may or may not be related to diverse pathologies of the human being.

One embodiment relates to a single dose therapeutic beverage, comprising: a beverage type liquid medium; and a plurality of spherical- and/or spheroidal-shaped particles dispersed within the liquid medium; wherein the plurality of the spherical- and/or spheroidal-shaped particles comprise at least one active pharmaceutical ingredient (API) disposed within the plurality of the spherical- and/or spheroidal-shaped particles; wherein the API partially permeates into the beverage type liquid medium from within the plurality of the spherical- and/or spheroidal-shaped particles so that an amount of the API is partitioned between the plurality of the spherical- and/or spheroidal-shaped particles and the liquid medium containing the particles; and wherein, upon administration to a subject, the therapeutic beverage is configured to provide an immediate dose of the API and a subsequent delayed release dose of the API.

In certain embodiments, in the single dose therapeutic beverage, the API can include a first API and a second API, different from the first API, and the first API is disposed within a first set of the plurality of spherical- and/or spheroidal-shaped particles and the second API is disposed with a second set of the plurality of spherical- and/or spheroidal-shaped particles.

The spherical- and/or spheroidal-shaped particles can have a diameter in a range from about 100 μm to about 6000 μm; more preferably, from about 500 μm to about 3000 μm; more preferably, from about 100 μm to about 3000 μm; more preferably, from about 2000 μm to about 3000 μm; or more preferably, from about 1000 μm to about 3000 μm.

Yet further embodiment relates to a single dose therapeutic beverage, comprising: a beverage container; a beverage type liquid medium disposed within the container; and a plurality of spherical- and/or spheroidal-shaped particles dispersed within the liquid medium; wherein the plurality of the spherical- and/or spheroidal-shaped particles comprise at least one active pharmaceutical ingredient (API) disposed within the plurality of the spherical- and/or spheroidal-shaped particles; wherein the spherical- and/or spheroidal-shaped particles have a diameter in a range from about 100 μm to about 6000 μm; wherein the beverage container contains between about 10-1000 mL of the single dose therapeutic beverage, or between about 10-500 mL of the single dose therapeutic beverage.

The most critical feature of the described single dose therapeutic formulations may be the equilibrium between the API and excipients inside the spheres, and the API and excipients in the liquid medium. That equilibrium permits to have a beverage that is not a syrup or a tablet, giving people a much easier to ingest and enjoyable pharmaceutical form.

In certain embodiments, at least one API can be disposed directly in the liquid medium of the described single dose therapeutic formulation or beverage. In this instance, the API from the particles may or may not permeate into the medium.

The percentage of APIs present in the liquid medium may be from about 10% to about 50% w/v and the percentage of APIs present in the plurality of the spherical- and/or spheroidal-shaped particles is from about 50% to about 90% w/w; or the percentage of APIs present in the liquid medium may be from about is from about 15% to about 35% w/v and the percentage of APIs present in the plurality of the spherical- and/or spheroidal-shaped particles is from about 65% to about 85% w/w; or the percentage of APIs present in the liquid medium may be about 30% w/v and the percentage of APIs present in the plurality of the spherical- and/or spheroidal-shaped particles is about 70% w/w.

The pharmaceutical formulations described herein show surprising advantages over the commercially available formulations. Indeed, the described formulations can be used to produce a pharmaceutical form, e.g., a single dose therapeutic beverage for immediate oral ingestion, offering an effective amount/concentration of the active ingredient(s), APIs either inside the spheres and/or spheroids that are part of the pharmaceutical formulation and/or in the aqueous phase of said pharmaceutical formulation. In addition, problems of stability and/or low solubility of the APIs are solved with the described formulations, and any type of interaction(s) between the APIs and the gelling agent(s) and/or emulsifiers and/or dispersants and/or other excipients present in the formulation are avoided with the present formulations.

The terms “active pharmaceutical ingredient,” “API”, “drug,” or “medicament” can be used interchangeably and refer to a substance that has a therapeutic effect. Throughout the description it will be understood that the reference to “API” refers to one or more active pharmaceutical ingredients or “APIs” present in the pharmaceutical formulation (i.e., in a single dose therapeutic formulation).

As used herein, the term “effective dose” refers to that concentration of the APIs in the single dose therapeutic formulation, which when administered to a subject is capable of producing the desired effect in a subject (i.e., pain reduction).

As used herein, the singular forms “a,” “an,” and “the” include plural reference unless the context clearly dictates otherwise. The term “and/or” means one or all of the listed elements or a combination of any two or more of the listed elements.

The terms “preferred” and “preferably” refer to embodiments described herein that may afford certain benefits, under certain circumstances. However, other embodiments may also be preferred, under the same or other circumstances.

Furthermore, the recitation of one or more preferred embodiments does not imply that other embodiments are not useful, and is not intended to exclude other embodiments from the scope of the invention.

As used herein, “around,” “about” or “approximately” shall generally mean within 20 percent, preferably within 10 percent, and more preferably within 5 percent of a given value or range. Numerical quantities given herein are approximate; meaning that the terms “around,” “about” or “approximately” can be inferred if not expressly stated. When the term “about” is used in describing a value or an endpoint of a range, the disclosure should be understood to include both the specific value and end-point referred to.

As used herein, the terms “comprising,” “including,” “having,” “containing,” “involving,” and the like are to be understood to be open-ended, i.e., to mean including but not limited to.

The terms “formulation” or “composition” or “beverage” are used herein to describe a formulation that includes gelled spherical- or spheroidal-shaped particles that comprise one or more APIs dissolved and/or dispersed in a liquid, dispersed in an oil or in oil form, or by forming an emulsion within the spherical- or spheroidal-shaped particles, and where said spherical- or spheroidal-shaped particles are suspended in an aqueous liquid suitable for immediate ingestion. The term refers to a comestible formulation that is suitable for oral ingestion by the subject (e.g., the human subject). In certain preferred embodiments, the composition described herein is an aqueous therapeutic beverage. In certain other embodiments, a component of the described formulation may be mixed into an aqueous beverage by the subject before consumption.

As used herein, the terms “effective amount” or “pharmaceutically effective amount” or “therapeutically effective amount” refer to the amount of the APIs to be administered orally to the subject in the single dose formulation described herein to trigger the desired effect without or causing minimal toxic adverse effect against the subject, and/or without undesirable side-effects. One skilled in the art should know that the effective amount can vary from one individual to another due to the external factors such as age, sex, diseased state, races, body weight, formulation of the composition, availability of other active ingredients in the formulation, and so on.

By “preventing” or “reducing the likelihood of” is meant reducing the severity, the frequency, and/or the duration of a condition or disorder or the symptoms thereof. For example, reducing headache.

By “treating” or “ameliorating” or “alleviating” is meant administering a composition for therapeutic purposes or administering treatment to a subject already suffering from a disorder to improve the subject's condition. By “treating a symptom and/or discomfort” or “ameliorating a symptom and/or discomfort” is meant that the symptom and/or discomfort of a condition or disorder (e.g., pain) and the symptoms associated with the condition or disorder are, e.g., alleviated, reduced, cured, or placed in a state of remission. As compared with an equivalent untreated control, such amelioration or degree of treatment is at least 2%, 5%, 10%, 20%, 30%, 40%, 50%, 60%, 70%, 80%, 90%, 95%, 99%, or 100%, as measured by any standard, suitable technique.

By “beverage” is meant a composition that is not in solid or gas form, such as a liquid or semi-liquid that is designed to enter into the mouth of a subject and be orally consumed or ingested. A beverage may be in a ready-to-drink liquid form (e.g., may be consumed without modification) or in a liquid, solid, or concentrated form (e.g., capsule or stick pack), which can be transformed into a ready-to-drink liquid form with an addition of another liquid (e.g., water), such as an instant beverage.

By “instant beverage” is meant that the described composition is in a form of a premix, which may be a dry powder (e.g., capsule or stick pack) of beverage flavor that can blend with the APIs and water and other solvents. Instant beverages premixes are available in powder, granules, and paste forms.

The term “beverage type liquid medium” refers to any liquid medium that is intended for consumption by a subject. Different types of beverage type liquid mediums include, but are not limited to water, flavored water, sparkling water, juice, energy drinks, tea (e.g., ice-tea), and others.

As described herein, the term “subject” is equivalent to the terms “individual” and “patient” whereby the terms can be used interchangeably. “Subject” means any animal belonging to any species. Examples of subjects include, but are not limited to, commercially bred animals such as birds (hens, ostriches, chickens, geese, partridges, etc.), rabbits, hares, domestic animals (dogs, cats, etc.), livestock such as sheep and goat livestock, pigs, wild boars, horses, ponies, etc., and cattle (bulls, oxen, etc.). In a particular embodiment, the subject is a mammal, preferably a primate, more preferably a human being of any race, sex or age.

The terms “dispose” or “disposed” in the context of disposing the API in the spherical- and/or spheroidal-shaped particles, refers to placing, distributing, or arranging the API in at least a portion of each particle, e.g., inside the particle, on the surface of the particle, or both.

The terms “inside” and “in” in reference to the particles, mean that the API is interior or in the middle of the particle in different form (emulsion, etc.), with a homogeneous distribution throughout the particle, where the geometric form of a particle is an “sphere” or an irregular sphere (“spheroidal”). For example, when the particles are micromatrix, the distribution is homogeneous and, in some embodiments, there may be APIs on the surface of the particle as well.

In an embodiment, described is an oral pharmaceutical dosage formulation that is, preferably, available in a single dose therapeutic beverage form, including an aqueous solution and spherical- or spheroidal-shaped particles, including the APIs, that are suspended in the aqueous solution. The particles can be prepared with gelling agents and comprise one or more APIs inside that can be dissolved and/or dispersed in a liquid, dispersed in an oil or in oil form, or forming an emulsion inside, which ensure a pharmaceutically acceptable release dissolution profile of API according to its particular requirement.

In another embodiment, described herein is an oral unit dose, easy to ingest, with a pleasant taste, avoiding the need for additional liquid, typically required for swallowing of medicines in a pill form.

In another embodiment, described herein are methods for preparing the spherical- or spheroidal-shaped particles comprising APIs, at the defined effective concentrations, sufficient to bring about the desired effect/treatment upon administration of a subject. Also, methods for preparing the single dose therapeutic formulation are provided, which have surprising advantages, allowing a better management (as compared to methods known in the art) of the manufacture process. For example—and unlike what is reported in the state of the art—it was found that is not necessary to dry the particles before incorporating them into the final aqueous solution, as described herein.

In certain embodiments, described herein is a pharmaceutical formulation, e.g., a single dose therapeutic formulation that may include at least one of the following components:
(i) an aqueous liquid, which may further comprise excipients;
(ii) one or more API(s), selected from. e.g., those with analgesic action, such as paracetamol, mefenamic acid; nonsteroidal anti-inflammatory pain relievers (NSAIDs), such as acetaminophen (analgesic), aspirin, naproxen, ibuprofen, mefenamic acid (analgesic); Cannabis derivatives, such as delta-9-tetrahydrocannabinol (THC) and cannabidiol (CBD); antiallergics, such as benzhydramine, chlorpheniramine, cetirizine and loratadine; expectorant, such as guaifenesin; antitussive, such as dextromethorphan HBr; active ingredients of therapeutic groups such as antibiotics, such as chloramphenicol, amoxicillin, cephradine; diuretics, such as hydrochlorothiazide, furosemide; hypotensives, such as losartan, telmisartan, enalapril, valsartan; polyvitamins; hypoglycemic agents, such as glibenclamide, metformin; antispasmodics, such as pargeverine, scopolamine, mebeverine; antidepressants, such asfluoxetine, imipramine, paroxetine; anti-ulcer drugs, such as omeprazole, panttoprazole and ranitidine, among others;
(iii) an emulsifying agent, preferably selected from lecithin, fatty acid diglycerides and fatty acid esters, such as glyceryl monostearate, glyceryl monopalmitate, glyceryl monooleate, monostearin, monopalmitin, monoolein, polysaccharides such as carrageenan, guar gum or agar-agar, gum tragacanth, gum Arabic, methyl cellulose and/or mixtures thereof;
(iv) an oily dispersing agent, preferably selected from canola oil, olive oil, almond oil, chia oil, corn oil, marigold oil, coconut oil, soybean oil, mineral oils, and/or a mixture thereof;
(v) a gelling agent, preferably selected from gellan gum, methyl cellulose, methyl ethyl cellulose, hydroxy propyl methyl cellulose, locust bean gum, carrageenan, sodium alginate, xanthan gum, gelatine, chitosan, derivatives of chitosan and/or mixtures thereof;
(vi) an agent for spherification, preferably selected from salts, as calcium chloride, calcium acetate, calcium citrate, calcium phosphate, monobasic calcium phosphate and calcium gluconolactate and/or mixtures thereof;
(vii) excipients, preferably selected from stabilizers, preservatives, antioxidants, viscosifying agents, sweeteners, colorants or coloring agents, flavoring agents, pH stabilizers and/or mixtures thereof.

Exemplary sweeteners include high fructose corn syrup, mannose, maltose, glucose polymers, sucrose (e.g., cane sugar or beet sugar), glucose, dextrose, lactose, galactose, fructose, polysaccharides (e.g., maltodextrins), rice syrup, honey, and natural fruit juices (e.g., orange juice, papaya juice, pineapple juice, apple juice, grape juice, apricot juice, pear juice, tomato juice, agave nectar, or cranberry juice). Additionally, non- or low-caloric sweeteners can be used. Examples of such non-caloric or low-caloric sweeteners include, but are not limited to, saccharin, cyclamates, acetosulfam, sorbitol, sucralose, xylitol, erythritol, Stevia, Stevia extract, L-aspartyl-L-phenyl-alanine ester (e.g., aspartame), L-aspartyl-D-alanine alkyl amides, L-aspartyl-L-1-hydroxymethylalkaneamide, and L-aspartyl-1-hydroxyethylalkaneamide.

Exemplary flavoring agents include natural and synthetic flavoring agents, including almond oil, amaretto oil, anethole, anise oil, benzaldehyde, blackberry, black walnut oil, blueberry, caraway, caraway oil, cardamom oil, cardamom seed, cherry juice, cherry syrup, cinnamon, cinnamon oil, cinnamon water, citric acid, citric acid syrup, clove oil, cocoa, coriander oil, dextrose, eriodictyon, ethyl acetate, ethyl vanillin, fennel oil, ginger, glucose, glycerin, glycyrrhiza, grape, honey, lavender oil, lemon oil, lime, mannitol, methyl salicylate, myristica oil, orange oil, orange peel, orange syrup, peppermint, peppermint oil, peppermint water, phenylethyl alcohol, pineapple, raspberry juice, raspberry syrup, rosemary oil, rose oil, rose water, sarsaparilla syrup, sorbitol, spearmint, spearmint oil, strawberry, sucrose, thyme oil, tolu balsam, tropical, vanilla, vanillin, and wild cherry syrup. Additional flavoring agents may be found in Food Chemicals Codex and Fenaroli's Handbook of Flavor Ingredients.

Small amounts of one or more coloring agents may be utilized in the described formulations. Coloring agents include, e.g., beta-carotene, riboflavin dyes, FD&C dyes (e.g., Yellow No. 5, Blue No. 1, Blue No. 2, and Red No. 40), FD&C lakes, chlorophylls and chlorophyllins, caramel coloring, annatto, cochineal, turmeric, paprika, and fruit, vegetable, and/or plant extracts (e.g., grape, black currant, aronia, carrot, beetroot, red cabbage, elderberry, and hibiscus extracts). The amount of coloring agent used will vary depending on the agents used in the composition and the color intensity desired in the finished product. The amount of coloring agent to be used can be readily determined by one skilled in the art.

Non-limiting examples of vitamins and minerals that may be included in the described formulations include, e.g., choline bitartate, niacinamide, thiamin, folic acid, d-calcium pantothenate, biotin, vitamin A, vitamin C, vitamin B1 hydrochloride, vitamin B2, vitamin B3, vitamin B6 hydrochloride, vitamin B12, vitamin D, vitamin E acetate, vitamin K, and salts of calcium, potassium, magnesium, zinc, iodine, iron, and copper. When included in the described composition, the composition contains at least 5, 10, 15, 20, 25, 30, 35, 40, 45, or 50% of the U.S. recommended daily intake (RDI) for such vitamins and minerals.

One or more preservatives may additionally be utilized in the described compositions. Exemplary preservatives include, for example, sorbate, benzoate, and polyphosphate preservatives (e.g., sorbic acid, benzoic acid, calcium sorbate, sodium sorbate, potassium sorbate, calcium benzoate, sodium benzoate, potassium benzoate, and mixtures thereof).

One or more antioxidant agents may also be included in the described formulation. Exemplary antioxidants include vitamin C and vitamin E; beta-carotene, lutein, or other carotenoids; cyanidin, delphinidin, malvidin, or other anthocyanidins; apigenin, luteolin, or other flavones; hesperitin, naringenin, or other flavonones; isorhamnetin, quercetin, kaempferol or other flavonols; and epigallocatechin-3-gallate, epicatechin, thearubigins, or other flavan-3-ols.

Additional components of the formulation described herein may include amino acids (e.g., leucine, isoleucine, lysine, methionine, phenylalanine, threonine, tryptophan, and valine), stimulants (e.g., caffeine), emulsifying agents, carbon dioxide (e.g., to carbonate a liquid composition), stabilizers, humectants, anticaking agents, or herbal extracts.

The term “emulsifying agent”, as used herein refers to a compound that allows the formation of an emulsion, by forming a thin film around the globules of dispersed phase, it is stable over time. The emulsifying agent may be present in the dosage form as a single compound or as a mixture of compounds, for example as an emulsion structure with coexisting water/oil or oil/water morphologies. Examples of emulsifying agents include water/fatty acid, sorbitan esters with either oleic, palmitic, or stearic acid and/or mixtures thereof.

The term “oil”, as used herein refers to an oily dispersing agent. The oil may be present in the dosage form as a single compound or as a mixture of compounds. Examples of oils include olive oil, almond oil, canola oil, chia oil, corn oil, marigold oil, coconut oil, soybean oil, mineral oils, and/or a mixture thereof.

The term “gelling agent”, as used herein refers to a compound that has the ability to form gels. The gelling agent may be present in the dosage form as a single compound or as a mixture of compounds. Examples of gelling agents include gellan gum, methyl cellulose, methyl ethyl cellulose, hydroxy propyl methyl cellulose, locust bean gum, carrageenan, sodium alginate, xanthan gum, gelatine, chitosan, derivatives of chitosan and/or mixtures thereof.

The term “agent for spherification”, as used herein refers to a chemical compound that promotes the partial gelation of a polymer, such as sodium alginate, chitosan, methyl cellulose, hydroxypropylmethyl cellulose, gellan gum and/or mixtures thereof, upon coming into contact with them, generating a crosslinked structure capable of to surround or contain other substances suspended or dissolved in the polymer. The agent for spherification may be present in the dosage form as a single compound or as a mixture of compounds. Examples of spherification agents include divalent cations selected from calcium salts such as calcium chloride, calcium acetate, calcium citrate, calcium phosphate, monobasic calcium phosphate and calcium gluconolactate and/or mixtures thereof.

The term “pharmaceutically acceptable excipient”, as used herein means that it is an acceptable excipient from the point of view of its toxicity. Examples of excipients include stabilizers, preservatives, antioxidants, viscosifying agents, preservatives, flavorings, sweeteners, colorants, flavorings, pH stabilizers and/or mixtures thereof, and will be known to those skilled in the art.

The term “emulsion”, as used herein refers to the mixture of the components before being incorporated by dripping into the solution that comprises the “spherification agent” or the “gelling agent”, depending on gelation protocol.

In certain embodiments, the described single dose therapeutic formulation does not contain any GMO ingredient(s).

In certain embodiments, the described single dose therapeutic formulation may be lactose free.

In certain embodiments, the described single dose therapeutic formulation may be diary free.

In certain embodiments, the described single dose therapeutic formulation may be soy free.

In certain embodiments, the described single dose therapeutic formulation may be gluten free.

In certain embodiments, the described single dose therapeutic formulation may not contain artificial sweeteners.

In certain embodiments, the described single dose therapeutic formulation may not contain artificial preservatives.

In certain embodiments, the described single dose therapeutic formulation may not contain artificial flavors.

In certain embodiments, the described single dose therapeutic formulation may not contain artificial colors.

Also, in certain embodiments, no refrigeration is necessary for the described single dose therapeutic formulation (e.g., can be stored in a cool, dry place).

Furthermore, the described single dose therapeutic formulation may not include irradiated ingredients.

In certain embodiments, the described single dose therapeutic formulation may be Kosher and/or Hala certified.

In certain embodiments, the described single dose therapeutic formulation may be vegetarian and/or vegan.

In certain embodiments, the described single dose therapeutic formulation may be NSF certified.

In certain embodiments, the described single dose therapeutic formulation may be free from peanuts, tree nuts, eggs, shellfish, and crustacean.

In certain embodiments, the described single dose therapeutic formulation may be a packaged beverage provided in a unit that contains between 10-1000 mL of the beverage. Alternatively, the described single dose therapeutic formulation may be a packaged beverage provided in a unit that contains between 10-500 mL of the beverage. Alternatively, the described single dose therapeutic formulation may be a packaged beverage provided in a unit that contains between 40 mL to 500 mL; more preferably about 50 mL, more preferably, about 350 mL, and alternatively, about 500 mL.

In certain embodiments, the beverage may be in a form of an instant beverage.

In certain embodiments, the components of the single dose therapeutic formulation described herein can include the following components (included is the concentration, because the volume will depend of the quantity of the API):

TABLE 1
Estimated ranges for a formulation,
referred to 100% total formulation)

| | Range % |
|---|---|
| Component | |
| Gelling agents (aqueous solution of gelling agents) | 1-3 (75-85) |
| Agents for spherification (e.g., calcium chloride) | 2.0-6.0 |
| Emulsifying agents | 15-25 |
| Excipients: | |
| Vitamin E | 0.30-0.60 |
| Potassium sorbate | 0.05-0.15 |
| methyl paraben | 0.10-0.25 |
| Dispersing agents | 15-25 |

The number of particles in the single dose therapeutic formulation described herein can vary depending on the desired dose of the API. For example, the number of particles in the single dose therapeutic formulation described herein can be 10 to 500. For example, 120 particles may be used for 140 mg of acetaminophen (F1); 160 particles for 280 mg of acetaminophen (F2); and 236 particles for 560 mg of acetaminophen (F3).

### Experimental Section

General Procedure for Obtaining Spherical- and Spheroidal-Shaped Gel Particles Containing APIs and Suspended in an Aqueous Medium

The procedures for obtaining spheroidal gel particles containing pharmacologically active ingredients or API(s), suspended in an aqueous medium, were carried out using modifications of gelation processes, which were previously reported in the literature. In summary, the processes consist of the induction of a sol-gel of a polymeric gelling agent in a liquid medium (Carolina Villarreal-Otalvaro and Jeannine M. Coburn, Fabrication Methods and Form Factors of Gellan Gum-Based, Materials for Drug Delivery and Anti-Cancer Applications,” https://doi.org/10.1021/acsbiomaterials. 1c00685; Eng-Seng Chan, “Preparation of Ca-alginate beads containing high oil content: Influence of process variables on encapsulation efficiency and bead properties,” Carbohydrate Polymers, 84 (2011) 1267-1275; Shan Zhao et al., “Preparation and optimization of calcium pectate beads for cell encapsulation,” Inc. J. Appl. Polym. Sci. (2017), 134, 45685; Tomasz Z Osmatek et al., “Gellan gum macrobeads loaded with naproxen: The impact of various naturally derived polymers on pH-dependent behavior,” Journal of Biomaterials Applications, 33(1):140-155 (2018)).

Types of Gelation Utilized

The procedures used in the experiments described herein include: I. Ionotropic gelation (IG), II. Gelation by complex formation with polyelectrolytes (polyelectrolytic gelation, PG), III. Reverse gelation by complex formation with polyelectrolytes (polyelectrolytic gelation, PG), IV. Gelation induced by temperature changes (thermal gelation, TG), and combinations of these. Other procedures of obtaining gels can also be used, such as ultrasound-induced gelation, cation-free cryogelation, ionotropic cryogelation, non-solvent-induced phase separation, C02-induced gelation, and other methods described in the art. Additionally, combinations of these methods can also be used, including combinations of any gelation method or procedure with the procedures of IG, PG and TG.

Surprisingly, during the gelation stage of the experiments described in the examples, three different types of gel particles with different morphology were obtained (see 1-111 below), depending on the manufacturing process: (a) homogeneous gel particles throughout their interior (referred to herein as “micromatrix” (i.e., spheres of homogeneous matrix that are formed by the addition of a single gelling agent, to obtain a micromatrix), (b) homogeneous gel particles throughout their interior with two gelling agents (referred to herein as “micromatrix” (i.e., spheres of homogeneous matrix that are formed by the addition of two gelling agents, to obtain a micromatrix); and (c) gel particles with a gelled wall and a liquid center, referred to herein as “microcapsules” (i.e, spheres of aqueous center surrounded by a layer of gelling agent, by reverse addition of the components, to obtain a microcapsule). In particular, microcapsules were, surprisingly, obtained only in the reverse gelation processes, while in the rest of the experiments only micromatrix were obtained.

I. Ionotropic Gelation of Gelling Agent on Agent for Spherification

Preparation of Liquid Medium A:

The amount of API to be used in these experiments was weighed, which corresponds to approximately 25% of the most commonly administered unit dose of the same. The amount of API is suspended or solubilized in oily or aqueous phase (oil or water, depending on whether the experiment is done with or without emulsion, respectively). The oily phase additionally contains an emulsifying agent. This suspension is mixed with a solution of gelling agent in water. If the experiment uses an oil phase to suspend or solubilize the API, the mixture is then emulsified by stirring, without temperature change. If the mixture is an emulsion, the volume ratio are from 1:1 to 1:4, preferably from 1:2 to 1:4, more preferably 1:4 or 1:2 oil:water mixtures to prepare the emulsion. In all cases, the liquid mixture obtained corresponds to liquid medium A of the experiment.

Preparation of Liquid Medium B:

A solution of agent for spherification in water. This solution corresponds to liquid medium B.

Preparation of Liquid Medium C:

A solution of agent for spherification in water. This solution corresponds to liquid medium C.

Gelation of Spherical- or Spheroidal-Shaped Particles with API:

The reported volume of liquid medium A, with or without emulsion, is dripped onto the reported volume of liquid medium B, at ambient temperature and pressure. Dripping is done using a nozzle (syringe, needle, or micropipette tip) with gauge range from 10 to 32 G, preferably from 10 to 27 G, more preferably 10, 18, 21 and/or 27 G. The formed particles are stirred in liquid medium B for about 10 minutes. Following the stirring step, the gel particles are filtered to separate them from the liquid, and are next suspended in liquid medium C.

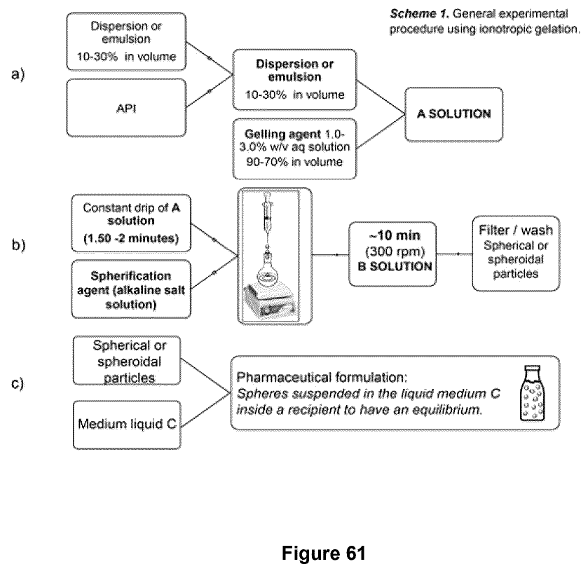

The process for preparing spherical- or spheroidal-shaped particles is shown in Scheme 1 (FIG. 61), which corresponds to the general experimental procedure for the examples of ionotropic gelation.

II. Gelation of Gelling Agent by Formation of Polyelectrolyte Complexes with Another Gelling Agent

Preparation of Liquid Medium A:

The amount of API to be used in the experiment is weighed, which corresponds to approximately 25% of the most commonly administered unit dose of the same. The amount of API is suspended or solubilized in oily or aqueous phase (oil or water, depending on whether the experiment is done with or without emulsion, respectively). Optionally, the oily phase additionally contains an emulsifying agent. This suspension is mixed with a solution of gelling agent in water. If the experiment uses an oil phase to suspend or solubilize the API, the mixture is then emulsified by stirring. If the mixture is an emulsion, the volume ratio of oil:water is 1:4 or 1:2 oil:water mixtures to prepare the emulsion. In all cases, the liquid mixture obtained corresponds to liquid medium A of the experiment.

Preparation of Liquid Medium B:

A solution of a second gelling agent and agent for spherification in water is prepared. This solution corresponds to liquid medium B.

Preparation of Liquid Medium C:

A solution of agent for spherification in water, CaCl2, sodium citrate among other excipients. This solution corresponds to liquid medium C.

Gelation of Spherical- and/or Spheroidal-Shaped Particles with API:

The reported volume of liquid medium A, with or without emulsion, is dripped onto the reported volume of liquid medium B, at ambient temperature and pressure. Dripping is done using a nozzle (syringe, needle, or micropipette tip) with 10, 18, 21 and/or 27-gauge. This procedure allows to obtain spherical and/or spheroidal particles with a size range between approximately 0.3 mm diameter and approximately 5 mm diameter. The formed particles are stirred in liquid medium B for about 10 minutes, with the stirring speed reported in each example. After the stirring step, gel particles are filtered to separate them from the liquid and are suspended in liquid medium C.

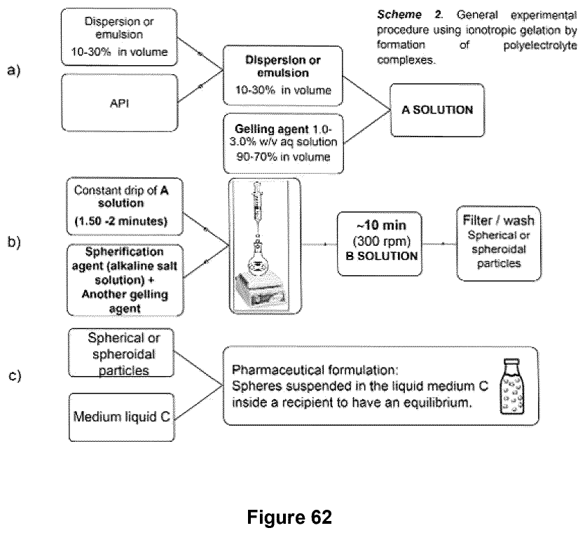

The process of preparing gel particles is shown in Scheme 2 (FIG. 62), which corresponds to the general experimental procedure for the examples of gelation by formation of polyelectrolyte complexes.

III. Reverse Gelation of a Gelling Agent and Another Gelling Agent by Formation of Polyelectrolyte Complexes

Preparation of Liquid Medium A:

The amount of API to be used in the experiment is weighed, which corresponds to approximately 25% of the most commonly administered unit dose of the same. The amount of API is suspended or solubilized in oily or aqueous phase (oil or water, depending on whether the experiment is done with or without emulsion, respectively). Optionally, the oily phase additionally contains an emulsifying agent.

This suspension is mixed with a solution of gelling agent and an agent for spherification in water. If the experiment uses an oil phase to suspend or solubilize the API, the mixture is then emulsified by stirring. If the mixture is an emulsion, the volume ratio of oil:water in these experiments is 1:4. In all cases, the liquid mixture obtained corresponds to liquid medium A of the experiment.

Preparation of Liquid Medium B:

A solution of another gelling agent in water is prepared. This solution corresponds to liquid medium B.

Preparation of Liquid Medium C:

A solution of an agent for spherification in water. This solution corresponds to liquid medium C.

Gelation of Spherical- or Spheroidal-Shaped Particles with API:

The reported volume of liquid medium A, with or without emulsion, is dripped onto the reported volume of liquid medium B, at ambient temperature and pressure. Dripping is done using a nozzle (syringe, needle, or micropipette tip) with 18 or 21 gauge. The formed particles are stirred in liquid medium B for about 10 minutes, with the stirring speed reported in each example. After the stirring step, the gel particles are filtered to separate them from the liquid and are suspended in liquid medium C.

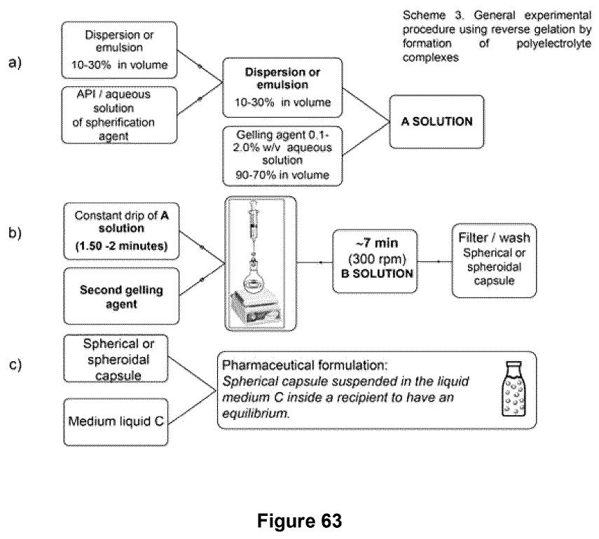

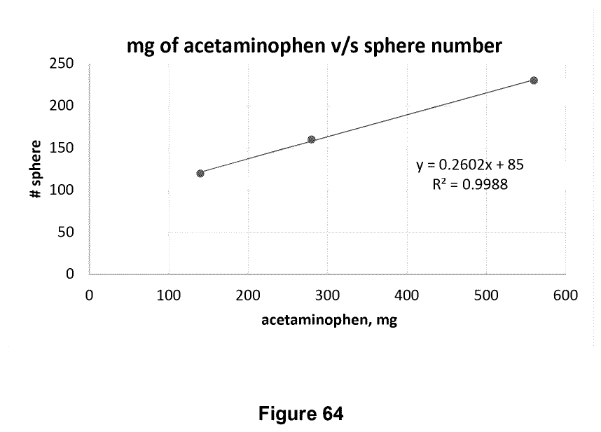

The process of preparing gel particles described herein is shown in Scheme 3 (FIG. 63), which corresponds to the general experimental procedure for the examples of reverse gelation by formation of polyelectrolyte complexes.

Obtaining Spherical or Spheroidal Particles

To obtain sphere- or spheroidal-shaped gel particles, a mechanical procedure was used, in which a liquid medium A is dropped, wherein A is a suspension or emulsion containing the API (APIs), gelling agent, alkali metal ion (alkali metal ions complex the gelling agent) and an emulsifying agent. This liquid medium A, is dropped to another liquid medium called B, which contains the agent that promotes esterification. The liquid media can be aqueous, non-aqueous, and/or mixed. During this procedure, the liquid medium B is stirred, for a suitable period of time to prevent the formation of aggregates of gel particles, and/or promote the formation of spheroidal shaped gel particles. The agitation of the liquid medium B can be achieved by any method, including mechanical, magnetic, thermal or ultrasonic agitation. The liquid medium B can be maintained in a constant temperature or pH range, by any method useful for these purposes. Liquid medium A can be stirred or emulsified during the process, and its temperature and/or pH can be controlled.

The formation of drops from the liquid medium A that are going to be deposited on the liquid medium B can be carried out using any method available for this described in the state of the art.

In certain embodiments, preferably, dripping a flow of a liquid stream that is extruded through one or more nozzles with fixed or variable diameter can be used. Drops can form spontaneously by gravitational effect, by overcoming the surface tension forces that keep the forming drop adhered to and suspended from the nozzle, while the size of the forming droplet increases, or it may be favoured or caused by other mechanisms, such as mechanical droplet cutting systems, liquid medium flow cutting or variation, nozzle(s) vibration systems, cutting by blowing or gas jets, cutting by temperature change, or combinations of these or other methods appropriate. Different nozzle shapes, sizes, materials, orientations, and droplet formation processes can be used to obtain different sizes and/or shapes of gel particles useful in the described methods. The flow rate of liquid medium A passing through the nozzles, the temperature of liquid mediums A and B, the height from which drops fall to liquid medium B, and other parameters of the dripping process can also be altered, to obtain different sizes and/or shapes of gel particles useful in the described methods.

Once a drop of liquid medium A has dropped onto liquid medium B, a spherical or spheroidal shaped gel particle is formed. During this formation process, the gel particle changes its mechanical properties and composition, including its shape, size, texture, hardness, resistance to deformation, resistance to rupture, concentration of species within the particle, and other similar properties. This change process occurs with composition-dependent formation kinetics and specific properties of each liquid medium A and liquid medium B used. The gel particle is left in suspension in the liquid medium B for a certain period of time (e.g., 5-20 min), until the particle obtains the desired mechanical and/or compositional property(s).

Obtaining Spherical- or Spheroidal-Shaped Particles with APIs

In certain embodiments, the APIs are incorporated into the final dosage form by incorporating them into the spherical or spheroidal particles, which is done before or during the gelation process. This incorporation is obtained by adding the APIs to the liquid medium A, before dripping it into liquid medium B to obtain the gel particles. The APIs can be incorporated into liquid A in any physical presentation: in crystalline or amorphous form, as powder, as microparticulate material or nanoparticles, or incorporated in liposomes or other nano and microcarriers, or in mixtures of the above. Importantly, the APIs with liquid medium A can form a solution, a suspension, a dispersion, a microdispersion, a colloidal suspension, an emulsion, a microemulsion, a liposomal suspension, or another type of mixture.

In certain embodiments, the liquid medium A can contain a mixture of miscible, immiscible, or partially miscible solvents. In particular, importantly, the liquid medium A can be a mixture of immiscible or partially miscible liquids that form an emulsion, a microemulsion, a liposomal suspension or other stable or pseudostable intimate mixture of immiscible or partially miscible solvents. APIs can be incorporated into the liquid medium A before, during or after the formation of the emulsion, the microemulsion, the liposomal suspension or any other stable or pseudostable intimate mixture of partially or totally immiscible solvents.

In particular, importantly, the liquid medium A may be a mixture of miscible, immiscible, or partially miscible solvents, in which one or more of the solvents are in an aqueous medium or constitute an aqueous medium, and one or more of the remaining solvents are in a non-aqueous medium or constitute a non-aqueous medium. In this situation, the emulsion, microemulsion, liposomal suspension or other stable or pseudo-stable intimate mixture of immiscible or partially miscible solvents that constitutes the liquid medium A, may be formed by an aqueous phase and another non-aqueous phase. Conveniently, the non-aqueous phase may be an oil phase or an oily phase.

In certain embodiments, APIs, in any of their presentations, can be incorporated into the liquid medium A in one of the phases or in both at the same time. Preferably, APIs may be previously incorporated into the liquid medium A, during or after forming an emulsion, microemulsion, liposomal suspension, or other stable or pseudo-stable intimate mixture of immiscible or partially miscible phases. In particular, it was surprisingly found that the most unexpected effects of the described formulations are obtained by incorporating the APIs into the liquid medium A contained in an oily phase and by forming an emulsion or microemulsion of oil-in-water type between the aqueous phase and the oily phase. The emulsion can be stable or unstable, and emulsifying agents can optionally be used to modify its stability over time. It should be noted that the effects observed with the described formulations and methods are not limited only to the case of stable or unstable emulsions.

Washing or Changing the Liquid Suspension Medium of the Spherical- or Spheroidal-Shaped Particles

Once the freshly gelled gel particle in liquid medium B has desired properties, it can remain in liquid medium B for an indefinite time, or it can be mechanically separated from liquid medium B, using any method available in the art. Preferably, the gel particles can be filtered, strained, or sieved to separate them from the liquid medium B. In certain embodiments, the liquid medium in which the gel particles are formed can be diluted or mixed with another liquid medium with or without gel particles, without separating the particles from the initial liquid medium.

As part of this procedure, the particles separated from an initial liquid medium may also be washed one or more times with another liquid medium before being incorporated into a final liquid medium. For this purpose, any method known in the art can be used. For example, the particles can be washed by retaining them on a screen or filter and passing a stream of washing liquid through the retained bed of particles.

Once separated from the liquid medium B, the gel particles can be successively contacted with one or more liquid mediums (aqueous, non-aqueous or mixed), in order to change their physical, mechanical or chemical composition properties, or in order to be stored as a suspension of particles in a final liquid medium. The liquid medium change procedure can be done with or without physical separation of the particles from their initial medium, including filtration, straining, screening, dilution, mixing with another liquid medium with or without particles, or any other suitable method.

As part of the media change and/or wash procedure, the particles may remain for fixed or variable periods in an intermediate liquid medium or may be washed for fixed or variable periods with one or more washing liquids, using any suitable method available in the state of the art. These times can be determined by the person skilled in the art and can be chosen to modify or maintain the physical or chemical properties on the gel particles, or in the liquid that contains them, until desired values are obtained (e.g., about 30 second to about 90 seconds).

Formulation of Spherical- or Spheroidal-Shaped Particles Suspended in a Liquid Medium

In an exemplary formulation described herein, the gel particles are suspended in a final liquid medium C, particularly in an aqueous liquid medium which may optionally comprise excipients or other components described previously. The APIs of interest is(are) contained in the gel particles and/or in the final liquid medium C, in a desired proportion, in order to obtain the pharmacological effect and/or the desired pharmacokinetic properties for each API in the final formulation, when consumed by a subject.

In certain embodiments, once the gel particles are suspended in a final liquid medium C, the APIs can permeate into the medium so that some of the API(s) are in the medium, while some still remain in the particles.

In particular, the formulation allows to obtain a modified release of the APIs:
the immediate release of a fraction of the total amount of APIs, available for absorption in the digestive system (i.e., first phase), and
the prolonged/extended release and/or or retarded release of the other/remaining fraction of the API.

The manufacture of the gel particles and the liquid medium C that contains them and the formulation of both, will be in accordance with the parameters established herein to obtain the desired release and bioavailability profiles.

Partition of API Between the Particles and the Final Liquid Medium of the Formulation

Surprisingly, when the APIs are introduced into a liquid medium A with an aqueous phase and a non-aqueous phase to form spheroidal gel particles with APIs, the partitioning of the API between the interior and the outside of the particles, differs (increases or decreases) with respect to the type of the API contained in gel particles formed from a liquid medium A with a single phase, particularly with a single aqueous phase.

Surprisingly, the direction of change of this variation (increase or decrease in the partition coefficient) is not intuitive and it is not possible to anticipate from the partition of the APIs between the pure constituent phases, but it directly depends on: (1) the type and concentrations of the gelling agent(s) used, (2) the composition of the phases used to form the liquid medium A, (3) the proportion between the phases used to form the liquid medium A, (4) the composition of the liquid medium B, (5) the concentration of the API used, (6) the chemical nature, the physical state and the physicochemical properties of the API used, (7) the composition and properties of the final liquid medium C, (8) the size and shape of the gel particles, and (9) the ratio of the volume of the gel particles to the total volume of the final formulation.

Considerations for the Procedure for Obtaining Spherical- or Spheroidal-Shaped Particles Containing APIs and Suspended in an Aqueous Medium

In the illustrative examples described herein, a method of gravitational dripping of a liquid medium A on a liquid medium B was used, the latter stirred with magnetic agitation, using a nozzle with a circular or elliptical cross-sectional area, through which the pass a given constant flow of liquid medium A, so as to form drops that fall into liquid medium B from a given height. For example, the nozzles used in these examples were laboratory syringes with different outer gauge diameters needles from 10 to 32-gauge, more preferably 10, 18, 21 and/or 27-gauge, and syringes of 10 mL without needles. In some experiments, cut micropipette tips were also used as nozzles in order to obtain different outlet diameters and change the size of the gel particles obtained during the gelation process. The parameters of dripping, temperature and agitation were chosen for all cases in order to promote the formation of essentially spherical and/or spheroidal gel particles, with homogeneous size, and without the formation of aggregates. The cases in which a spherical and/or spheroidal shape of the particles was not achieved, or their size was not homogeneous despite these precautions, are clearly identified and shown in the results.

In the examples described herein, the APIs used in the pharmaceutical formulation(s) were contained in the liquid medium A, which is dripped on the liquid medium B to form the spherical or spheroidal particles. The APIs can be incorporated into liquid A in crystalline or amorphous form, as a powder, as microparticulate material or nanoparticles, or incorporated in liposomes or other nano and microcarriers, or in mixtures of the above. The API with liquid medium A can form a solution, suspension, dispersion, microdispersion, colloidal suspension, emulsion, microemulsion, or other mixture. The experiments demonstrated that said physical form of the API incorporated in the liquid medium A depends on the physicochemical and molecular properties of the API and the liquid medium A and can, surprisingly, change by changing the relative amounts of API and liquid medium A and the composition of the liquid medium A, as shown in the examples. These changes can be used to manage, adjust and optimize the release of the API from the spherical- and/or spheroidal-shaped particles and the relationship between the fractions of API contained in the gel particles and the final liquid medium of the formulation, which is useful to effects and applications of pharmaceutical formulations described herein.

When the liquid medium A is made up of two phases (an aqueous phase and another oily phase), in the examples presented, the two phases of liquid A are emulsified previously in the gelation step. To demonstrate the unexpected and surprising effects of the described formulations and methods, and how the release of the API can be selected and controlled in the final formulation, the API used in each example is first incorporated into the oil phase, which is homogenized before the oil phase is added oil phase to the water phase and emulsify the mixture. Thus, the API in the examples was preferably used solubilized or suspended in the oily phase, then the aqueous and oily phases of liquid medium A were emulsified and finally the emulsion was used to obtain spheroid gel particles containing API, described previously. Comparatively, some examples used a liquid medium A consisting only of the aqueous phase, with the API in solution or suspension in said aqueous phase, without an oily phase and therefore without the formation of an emulsion.

In the different examples described herein, the spherical or spheroidal gel particles with APIs were kept in the liquid medium B for a time from 5 minutes to 1 hour or more. After this period, the gel particles, in some cases, were separated from the liquid medium B by filtration (straining), and were immediately introduced into a liquid washing medium, or immediately washed using a liquid washing medium, for a period of time, preferably, e.g. for less than one minute. The liquid wash medium was distilled, demineralized and/or sterilized water and/or the liquid wash medium also can be the liquid medium C. After the wash period, the particles were introduced into a given volume of final liquid medium C. In other examples, the gel particles were separated from the medium liquid B by filtration (straining), and were introduced into the final liquid medium C.

All the examples used a certain proportion between the total volumes of the liquid media used in each example. To make comparative measurements in similar settings, many of the examples used the same volume ratios, without this implying, involving or being interpreted as a limitation of the scope and spirit of the described formulations and methods. The volume ratio of the liquid media used is one of the factors that can be used to manage, adjust, and optimize the release of the API from the spheroidal gel particles. On the other hand, it may be also possible to optimize the ratio between the fraction of API contained in the gel particles and the final liquid medium of the formulation. Therefore, these factors are useful to the purposes and applications of the pharmaceutical formulations described herein.

The formation of the spheroidal gel particles and experimental conditions studied in the examples are described below. For these experiments, different APIs were used with different physical and chemical properties, which should be interpreted in a non-limiting manner. The APIs used were: (a) acetaminophen (paracetamol), (b) ibuprofen, (c) naproxen sodium, (d) mefenamic acid, (e) acetyl salicylic acid, (f) chloramphenicol, (g) chlorphenamine maleate, (h) cannabinoidiol (CBD), and (i) 9Δ-tetrahydrocannabinol (THC). The first five compounds are pain relievers, the sixth is an antibiotic, the seventh is an antihistamine, and the last two are secondary metabolites of the hemp plant extracted into oil.

Acetaminophen is representative of molecules that are poorly soluble in water or in oily media, although it is more lipophilic (log P=4.6). Naproxen sodium is soluble in water and, in its protonated acid form, is not very soluble in the oil phase (log P=2.7). Mefenamic acid is very poorly soluble in water, where it can be negatively charged by deprotonation, and it is also not soluble in the oil phase (log P=4.2). Chloramphenicol is very soluble in water and it slightly soluble in oily media (log P=1.1). Chlorphenamine maleate is very soluble in water and it can acquire a positive charge by protonation, although it is also soluble in the oil phase (log P=3.7). Acetyl salicylic acid is soluble in water and it slightly soluble in oily media (log P=1.18). And finally, CBD and THC are very lipophilic molecules, highly soluble in oil and very slightly soluble in water (log P=7.75 and 7.26, respectively).

In each of the experiments, different mixes were tested in relation to the different gelation mechanisms involved. These examples are divided according to the composition of the liquid media and according to the type of encapsulation tests. Thus, for micromatrix-type encapsulation, the called liquid A is the emulsion or dispersion, which contains the API or APIs, emulsifying agent and gelling agent. While the called liquid B, is the solution of the agent that promotes the esterification that contains or does not contain a second gelling agent. On the other hand, in microcapsule-type encapsulation, the called liquid A is the emulsion or dispersion, which contains the API or APIs, gelling agent, emulsifying agent and the agent that promotes esterification to the gelling agent. While liquid B is the solution that contains an emulsifying agent.

To obtain the dimensions of the spheres, one representative spheres number were selected. The selected spheres were isolated by experiment and the dimensions and shape of the spheres, were performed by Scanning electron microscopy (SEM) observations and were performed at the Andean Geothermal Center of Excellence (CEGA), Universidad de Chile, Santiago, Chile, using a FEI Quanta 250 SEM equipped with secondary electron (SE), backscattered electron (BSE) and X-ray energy-dispersive spectrometry (EDS) detectors. The analytical parameters measured were the following: accelerating voltage of 15-20 kV, filament current ˜80 μA, beam intensity of 1 nA, takeoff angle ˜35°, a spot size of 4-5 μm in diameter, and a working distance of ˜10 mm. The INCA software was used for measurements and data processing.

To the measurements of APIs release, the APIs concentrations were determined spectrophotometrically, by sampling in a Thermo Spectronic Genesys 10 UV equipment. Each of the measurements were performed in duplicate or more trials. The wavelengths occupied will depend on each API tested (for example, 243 nm for acetaminophen, 230 nm for naproxen).

To evaluate each of the experiments carried out and adjust the concentration points during the release experiments, various methods were used: zero order models, first order, Higuchi, Hixson-Crowell, square root of the mass, root 3/2 of the mass, Weibull and Korsmeyer-Peppas. The model that best fits the concentration points during the release experiments was a Weibull model (Vasiliki Papadopoulou, Kosmas Kosmidis, Marilena Vlachou, Panos Macheras, “On the use of the Weibull function for the discernment of drug release mechanisms,” International Journal of Pharmaceutics 309 (2006) 44-50).

To measure APIs controlled release dosage forms, dissolution essays were made on ERWEKA DT light Series delivers by simple dissolution testing with USP 2 paddle method. The DTs is equipped with 8 test station and a fixed drive head at 37° C. and paddle 50 RPM. The described forms were compared with 4 commercial forms: 1) Panadol® (500 mg, coated tablet), 2) Paracetamol® (Laboratorio Chile, 500 mg, pregelatinized starch), 3) Daytime Cold® and Flu Relief® (325 mg acetaminophen, liquid capsule) and 4) TAPSIN® chewable (360 mg as 3 pills of 120 mg each one). Three different simulated pHs were tested: simulated intestinal fluid (pH 6.8), simulated duodenum fluid (pH 5.6) and stomach gastric fluid (pH 1.2) in 500 mL of buffer. The buffers were prepared according to normative USP41-NF36_4492.

### EXAMPLES

For the purpose of illustrating the described formulations and methods, the following examples are provided, which should be interpreted in a non-limiting manner.

One skilled in the art will appreciate that routine variations and modifications may be made to the following examples, without exceeding the scope of the described formulations and methods.

A. Examples with Acetaminophen as API

In the following examples acetaminophen was used as API contained within the spherical particles or gel spheroids obtained according to the described methods.

### Example 1

120 mg of acetaminophen was suspended in 1.2 mL of water. This suspension was mixed with 4.8 mL of a 2.5% w/v alginate solution. Once the mixture (liquid medium A) was homogenized, it was added dropwise from a height of 6-10 cm over 24 mL of a CaCl₂ solution at 3% w/v (liquid medium B), using a 10 mL syringe with a 21-gauge needle. The gel particles formed were stirred for approximately 10 minutes in liquid medium B. After this time, the particles were filtered to separate them from the liquid and were suspended in 24 mL of a 20 mM CaCl₂ solution (liquid medium C).

### Example 2

120 mg of acetaminophen was suspended in 1.2 mL of canola oil with 1% v/v liquid soy lecithin. This suspension was mixed with 4.8 mL of a 2.5% w/v alginate solution. The mixture of oily phase and aqueous phase was then emulsified by stirring. The resulting emulsion (liquid medium A) was added dropwise from a height of 6-10 cm over 24 mL of a 3% w/v CaCl₂ solution (liquid medium B), using a 10 mL syringe with a 21-gauge needle. The gel particles formed were stirred for approximately 10 minutes in liquid medium B. After this time, the particles were filtered to separate them from the liquid and are suspended in 24 mL of a 20 mM CaCl₂ solution (liquid medium C).

### Example 3

120 mg of acetaminophen was suspended in 1.2 mL of water. This suspension was mixed with 4.8 mL of a 2.5% w/v alginate solution. Once the mixture (liquid medium A) was homogenized, it was added dropwise from a height of 6-10 cm over 24 mL of a CaCl₂ solution at 3% w/v (liquid medium B), using a 10 mL syringe with an 18-gauge needle. The gel particles formed were stirred for approximately 10 minutes in liquid medium B. After this time, the particles were filtered to separate them from the liquid and are suspended in 24 mL of a 20 mM CaCl₂ solution (liquid medium C).

### Example 4

120 mg of acetaminophen was suspended in 1.2 mL of canola oil with 1% v/v liquid soy lecithin. This suspension was mixed with 4.8 mL of a 2.5% w/v alginate solution. The mixture of oily phase and aqueous phase was then emulsified by stirring. The resulting emulsion (liquid medium A) was added dropwise from a height of 6-10 cm over 24 mL of a 3% w/v CaCl₂ solution (liquid medium B), using a 10 mL syringe with an 18-gauge needle. The gel particles formed were stirred for approximately 10 minutes in liquid medium B. After this time, the particles were filtered to separate them from the liquid and were suspended in 24 mL of a 20 mM CaCl₂ solution (liquid medium C).

### Example 5

120 mg of acetaminophen was suspended in 1.2 mL of water. This suspension was mixed with 4.8 mL of a 2.5% w/v alginate solution. Once the mixture (liquid medium A) was homogenized, it was added dropwise from a height of 6-10 cm over 24 mL of a solution comprises of 4.8 mL of a 1.0% w/v chitosan solution and 19.2 mL of a 3% w/v CaCl₂ solution (liquid medium B), using a 10 mL syringe with a 21-gauge needle. The gel particles formed were stirred for approximately 10 minutes in liquid medium B. After this time, the particles were filtered to separate them from the liquid and are suspended in 24 mL of a 20 mM CaCl₂ solution (liquid medium C).

### Example 6

120 mg of acetaminophen was suspended in 1.2 mL of water. This suspension was mixed with 4.8 mL of a 2.5% w/v alginate solution. Once the mixture (liquid medium A) was homogenized, it was added dropwise from a height of 6 cm over 24 mL of a solution comprises of 4.8 mL of a 1.0% w/v chitosan solution and 19.2 mL of a 3% w/v CaCl₂ solution (liquid medium B), using a 10 mL syringe with an 18-gauge needle. The gel particles formed were stirred for approximately 10 minutes in liquid medium B. After this time, the particles were filtered to separate them from the liquid and are suspended in 24 mL of a 20 mM CaCl₂ solution (liquid medium C).

### Example 7

120 mg of acetaminophen was suspended in 1.2 mL of canola oil with 1% v/v liquid soy lecithin. This suspension was mixed with 4.8 mL of a 2.5% w/v alginate solution. The mixture of oily phase and aqueous phase was then emulsified by stirring. The resulting emulsion (liquid medium A) was added dropwise from a height of 6 cm over 24 mL of a solution comprises of 4.8 mL of a 1.0% w/v chitosan solution and 19.2 mL of a CaCl₂ solution at 3% w/v (liquid medium B), using a 10 mL syringe with an 18-gauge needle. The gel particles formed were stirred for approximately 10 minutes in liquid medium B. After this time, the particles were filtered to separate them from the liquid and were suspended in 24 mL of a 20 mM CaCl₂ solution (liquid medium C).

### Example 8

120 mg of acetaminophen was suspended in 1.2 mL of water. This suspension was mixed with 4.8 mL of a 2% w/v gellan gum solution. Once the mixture (liquid medium A) was homogenized, it was heated to 40° C. and added dropwise from a height of 6-10 cm over 24 mL of a 3% w/v CaCl₂ solution (liquid medium B), using a 10 mL syringe with an 18-gauge needle, always maintaining the temperature of liquid medium A at 40° C. throughout the drip. The gel particles formed were stirred for approximately 10 minutes in liquid medium B. After this time, the particles were filtered to separate them from the liquid and were suspended in 24 mL of a 20 mM CaCl₂ solution (liquid medium C).

### Example 9

120 mg of acetaminophen was suspended in 1.2 mL of water. This suspension was mixed with 4.8 mL of a 2% w/v gellan gum solution. Once the mixture (liquid medium A) was homogenized, it was heated to 40° C. and was added dropwise from a height of 6-10 cm over 24 mL of a 3% w/v CaCl₂ solution (liquid medium B), using a 10 mL syringe with an 18-gauge needle, always maintaining the temperature of liquid medium A at 40° C. throughout the drip. The gel particles formed were stirred for approximately 10 minutes in liquid medium B. After this time, the particles were filtered to separate them from the liquid and were suspended in 24 mL of a 20 mM CaCl₂ solution (liquid medium C).

### Example 10

120 mg of acetaminophen was suspended in 1.2 mL of canola oil with 1% v/v liquid soy lecithin. This suspension was mixed with 4.8 mL of a 2% w/v gellan gum solution. Next, the mixture of oily phase and aqueous phase was emulsified by stirring, heating it to 40° C. The resulting emulsion (liquid medium A) was added dropwise from a height of 6 cm over 24 mL of a 3% w/v CaCl₂ solution (liquid medium B) at room temperature, using a 10 mL syringe with an 18-gauge needle, always maintaining the temperature of the liquid medium A at 40° C. throughout the dripping. The gel particles formed were stirred for approximately 10 minutes in liquid medium B. After this time, the particles were filtered to separate them from the liquid and were suspended in 24 mL of a 20 mM CaCl₂ solution (liquid medium C).

### Example 11

120 mg of acetaminophen was suspended in 1.2 mL of water. This suspension was mixed with 4.8 mL of a 1.5% w/v gellan gum solution. Once the mixture (liquid medium A) was homogenized, it was heated to 40° C. and was added dropwise from a height of 6-10 cm over 24 mL of a 3% w/v CaCl₂ solution (liquid medium B), using a 10 mL syringe with an 18-gauge needle, always maintaining the temperature of liquid medium A at 40° C. throughout the drip. The gel particles formed were stirred for approximately 10 minutes in liquid medium B. After this time, the particles were filtered to separate them from the liquid and were suspended in 24 mL of a 20 mM CaCl₂ solution (liquid medium C).

### Example 12

120 mg of acetaminophen was suspended in 1.2 mL of canola oil with 1% v/v liquid soy lecithin. This suspension was mixed with 4.8 mL of a 1.5% w/v gellan gum solution. Next, the mixture of oily phase and aqueous phase was emulsified by stirring, heating it to 40° C. The resulting emulsion (liquid medium A) was added dropwise from a height of 6 cm over 24 mL of a 6% w/v CaCl₂ solution (liquid medium B) at room temperature, using a 10 mL syringe with an 18-gauge needle, always maintaining the temperature of the liquid medium A at 40° C. throughout the dripping. The gel particles formed were stirred for approximately 10 minutes in liquid medium B. After this time, the particles were filtered to separate them from the liquid and were suspended in 24 mL of a 20 mM CaCl₂ solution (liquid medium C).

### Example 13

120 mg of acetaminophen was suspended in 1.2 mL of water. This suspension was mixed with 4.8 mL of a 2% w/v gellan gum solution. Once the mixture (liquid medium A) was homogenized, it was heated to 40° C. and was added dropwise over 24 mL of a solution comprises of 4.8 mL of a 1.0% chitosan and 19.2 mL of a 6% w/v CaCl₂ solution (liquid medium B) at room temperature, using a 10 mL syringe with an 18-gauge needle, always maintaining the temperature of liquid medium A at 40° C. throughout the drip. The gel particles formed were stirred for approximately 10 minutes in liquid medium B. After this time, the particles were filtered to separate them from the liquid and were suspended in 24 mL of a 20 mM CaCl₂ solution (liquid medium C).

### Example 14

120 mg of acetaminophen was suspended in 1.2 mL of canola oil with 1% v/v liquid soy lecithin. This suspension was mixed with 4.8 mL of a 2% w/v gellan gum solution. Next, the mixture of oily phase and aqueous phase was emulsified by stirring, heating it to 40° C. The resulting emulsion (liquid medium A) was added dropwise from a height of 6 cm over 24 mL of a solution comprises of 4.8 mL of a 1.0% w/v chitosan solution and 19.2 mL of a 6% w/v CaCl₂) solution (liquid medium B) at room temperature, using a 10 mL syringe with an 18-gauge needle, always maintaining the temperature of liquid medium A at 40° C. throughout the drip. The gel particles formed were stirred for approximately 10 minutes in liquid medium B. After this time, the particles were filtered to separate them from the liquid and were suspended in 24 mL of a 20 mM CaCl₂ solution (liquid medium C).

### Example 15

120 mg of acetaminophen was suspended in 1.2 mL of water. This suspension was mixed with 4.8 mL of a 5% w/v pectin solution. Once the mixture (liquid medium A) was homogenized, it was heated to 40° C. and was added dropwise from a height of 6-10 cm over 24 mL of a 6% w/v CaCl₂ solution (liquid medium B), using a 10 mL syringe with a 18-gauge needle, always maintaining the temperature of liquid medium A at 40° C. throughout the drip. The gel particles formed were stirred for approximately 10 minutes in liquid medium B. After this time, the particles were filtered to separate them from the liquid and were suspended in 24 mL of a 20 mM CaCl₂ solution (liquid medium C).

### Example 16

120 mg of acetaminophen was suspended in 1.2 mL of canola oil with 1% v/v liquid soy lecithin. This suspension was mixed with 4.8 mL of a 5% w/v pectin solution. Once the mixture (liquid medium A) was homogenized, it was heated to 40° C. and was added dropwise over 24 mL of a solution comprises of 4.8 mL of a 1.0% chitosan and 19.2 mL of a 6% w/v CaCl₂ solution (liquid medium B) at room temperature using a 10 mL syringe with a 21-gauge needle, always maintaining the temperature of the liquid medium A at 40° C. throughout the dripping. The gel particles formed were stirred for approximately 10 minutes in liquid medium B. After this time, the particles were filtered to separate them from the liquid and were suspended in 24 mL of a 20 mM CaCl₂ solution (liquid medium C).

### Example 17

120 mg of acetaminophen was suspended in 1.2 mL of water. This suspension was mixed with 4.8 mL of a 5.0% w/v pectin solution. Once the mixture (liquid medium A) was homogenized, it was heated to 40° C. and was added dropwise over 24 mL of a solution comprises of 4.8 mL of a 1.0% chitosan and 19.2 mL of a 6% w/v CaCl₂ solution (liquid medium B) at room temperature, using a 10 mL syringe with a 21-gauge needle, always maintaining the temperature of liquid medium A at 40° C. throughout the drip. The gel particles formed were stirred for approximately 10 min in liquid medium B. After this time, the particles were filtered to separate them from the liquid and were suspended in 24 mL of a 20 mM CaCl₂ solution (liquid medium C).

### Example 18

120 mg of acetaminophen was suspended in 1.2 mL of water. This suspension was mixed with 4.8 mL of a solution comprises of 60% of a 2.5% w/v alginate solution and 40% of a 2% w/v gellan gum solution. Once the mixture (liquid medium A) was homogenized, it was heated to 40° C. and it was added dropwise from a height of 6 cm, to room temperature, over 24 mL of a 6% w/v CaCl₂ solution (liquid medium B), using a 10 mL syringe with an 18-gauge needle, always maintaining the temperature of liquid medium A at 40° C. throughout the drip. The gel particles formed were stirred for approximately 10 minutes in liquid medium B. After this time, the particles were filtered to separate them from the liquid and were suspended in 24 mL of a 20 mM CaCl₂ solution (liquid medium C).

### Example 19

120 mg of acetaminophen was suspended in 1.2 mL of a 7.5% w/v CaCl₂ solution. This suspension was mixed with 4.8 mL of a 1.0% w/v chitosan solution. Once the mixture (liquid medium A) was homogenized, it was added dropwise from a height of 6 cm over 24 mL of a 2.5% w/v alginate solution (liquid medium B), using a 10 mL syringe with an 18-gauge needle. The gel particles formed were stirred for approximately 7 minutes in liquid medium B. After this time, the particles were filtered to separate them from the liquid and were suspended in 24 mL of a 20 mM CaCl₂ solution (liquid medium C).

### Example 20

120 mg of acetaminophen was suspended in 1.2 mL of a 7.5% w/v CaCl₂ solution. This suspension was mixed with 4.8 mL of a 1.0% w/v chitosan solution. Once the mixture (liquid medium A) was homogenized, it was added dropwise from a height of 6 cm over 72 mL of a 0.83% w/v alginate solution (liquid medium B), using a 10 mL syringe with an 18-gauge needle. The gel particles formed were stirred for approximately 7 minutes in liquid medium B. After this time, the particles were filtered to separate them from the liquid and were suspended in 24 mL of a 20 mM CaCl₂ solution (liquid medium C).

### Example 21

120 mg of acetaminophen was suspended in 1.2 mL of a 3% w/v CaCl₂ solution. This suspension was mixed with 4.8 mL of a 1.0% w/v chitosan solution. Once the mixture (liquid medium A) was homogenized, it was added dropwise from a height of 6 cm over 72 mL of a 0.83% w/v alginate solution (liquid medium B), using a 10 mL syringe with an 18-gauge needle. The gel particles formed were stirred for approximately 7 minutes in liquid medium B. After this time, the particles were filtered to separate them from the liquid and were suspended in 24 mL of a 20 mM CaCl₂ solution (liquid medium C).

### Example 22

100 mg of acetaminophen was suspended in 1.2 mL of water. This suspension was mixed with 4.8 mL of a 2.5% w/v alginate solution. Once the mixture (liquid medium A) was homogenized, it was added dropwise from a height of 6-10 cm over 24 mL of a CaCl₂ solution at 3% w/v (liquid medium B), using a 10 mL syringe with a 27-gauge needle. The gel particles formed were stirred for approximately 10 minutes in liquid medium B. After this time, the particles were filtered to separate them from the liquid and were suspended in 24 mL of a 20 mM sodium citrate solution (liquid medium C).

### Example 23

100 mg of acetaminophen was suspended in 1.2 mL of canola oil with 1% v/v liquid soy lecithin. This suspension was mixed with 4.8 mL of a 2.5% w/v alginate solution. The mixture of oily phase and aqueous phase was then emulsified by stirring. The resulting emulsion (liquid medium A) was added dropwise from a height of 6-10 cm over 24 mL of a 3% w/v CaCl₂) solution (liquid medium B), using a 10 mL syringe with a 27-gauge needle. The gel particles formed were stirred for approximately 10 minutes in liquid medium B. After this time, the particles were filtered to separate them from the liquid and were suspended in 24 mL of a 20 mM sodium citrate solution (liquid medium C).

### Example 24

100 mg of acetaminophen was suspended in 1.2 mL of water. This suspension was mixed with 4.8 mL of a 2.5% w/v alginate solution. Next, the mixture of oily phase and aqueous phase was emulsified by stirring, heating it to 40° C. The resulting emulsion (liquid medium A) was added dropwise from a height of 6 cm over 24 mL of a solution comprises of 4.8 mL of a 1.0% w/v chitosan solution and 19.2 mL of a 6% w/v CaCl₂ solution (liquid medium B) at room temperature, using a 10 mL syringe with a 27-gauge needle, always maintaining the temperature of liquid medium A at 40° C. throughout the drip. The gel particles formed were stirred for approximately 10 minutes in liquid medium B. After this time, the particles were filtered to separate them from the liquid and were suspended in 24 mL of a 20 mM sodium citrate solution (liquid medium C).

### Example 25

100 mg of acetaminophen was suspended in 1.2 mL of canola oil with 1% v/v liquid soy lecithin. This suspension was mixed with 4.8 mL of a 2.5% w/v alginate solution. Next, the mixture of oily phase and aqueous phase was emulsified by stirring, heating it to 40° C. The resulting emulsion (liquid medium A) was added dropwise from a height of 6 cm over 24 mL of a solution comprises of 4.8 mL of a 1.0% w/v chitosan solution and 19.2 mL of a 6% w/v CaCl₂ solution (liquid medium B) at room temperature, using a 10 mL syringe with a 27-gauge needle, always maintaining the temperature of liquid medium A at 40° C. throughout the drip. The gel particles formed were stirred for approximately 10 minutes in liquid medium B. After this time, the particles were filtered to separate them from the liquid and were suspended in 24 mL of a 20 mM sodium citrate solution (liquid medium C).

### Example 26

100 mg of acetaminophen was suspended in 1.2 mL of water. This suspension was mixed with 4.8 mL of a 2.5% w/v alginate solution. Once the mixture (liquid medium A) was homogenized, it was added dropwise from a height of 6-10 cm over 24 mL of a CaCl₂ solution at 3% w/v (liquid medium B), using a 10 mL syringe with a 10-gauge needle. The gel particles formed were stirred for approximately 10 minutes in liquid medium B. After this time, the particles were filtered to separate them from the liquid and were suspended in 24 mL of a 20 mM sodium citrate solution (liquid medium C).

### Example 27

100 mg of acetaminophen was suspended in 1.2 mL of canola oil with 1% v/v liquid soy lecithin. This suspension was mixed with 4.8 mL of a 2.5% w/v alginate solution. Once the mixture (liquid medium A) was homogenized, it was added dropwise from a height of 6-10 cm over 24 mL of a CaCl₂ solution at 3% w/v (liquid medium B), using a 10 mL syringe with a 10-gauge needle. The gel particles formed were stirred for approximately 10 minutes in liquid medium B. After this time, the particles were filtered to separate them from the liquid and were suspended in 24 mL of a 20 mM sodium citrate solution (liquid medium C).

### Example 28

120 mg of acetaminophen was suspended in 1.2 mL of water. This suspension was mixed with 4.8 mL of a 2.5% w/v alginate solution. The resulting emulsion (liquid medium A) was added dropwise from a height of 6 cm over 24 mL of a solution comprises of 4.8 mL of a 1.0% w/v chitosan solution and 19.2 mL of a 3% w/v CaCl₂ solution (liquid medium B) at room temperature, using a 10 mL syringe with a 10-gauge needle, always maintaining the temperature of liquid medium A at 40° C. throughout the drip. The gel particles formed were stirred for approximately 10 minutes in liquid medium B. After this time, the particles were filtered to separate them from the liquid and were suspended in 24 mL of a 20 mM sodium citrate solution (liquid medium C).

### Example 29

120 mg of acetaminophen was suspended in 1.2 mL of canola oil with 1% v/v liquid soy lecithin. This suspension was mixed with 4.8 mL of a 2.5% w/v alginate solution. The mixture of oily phase and aqueous phase was then emulsified by stirring. The resulting emulsion (liquid medium A) was added dropwise from a height of 6 cm over 24 mL of a solution comprises of 4.8 mL of a 1.0% w/v chitosan solution and 19.2 mL of a 6% w/v CaCl₂ solution (liquid medium B) at room temperature, using a 10 mL syringe with a 10-gauge needle, always maintaining the temperature of liquid medium A at 40° C. throughout the drip. The gel particles formed were stirred for approximately 10 minutes in liquid medium B. After this time, the particles were filtered to separate them from the liquid and were suspended in 24 mL of a 20 mM sodium citrate solution (liquid medium C).

### Example 30

100 mg of acetaminophen was suspended in 1.2 mL of water. This suspension was mixed with 6 mL of 4 mL 2.5% w/v alginate solution and 2 mL 1.5% w/v starch. Once the mixture (liquid medium A) was homogenized, it was added dropwise from a height of 6-10 cm over 24 mL of a CaCl₂ solution at 3% w/v (liquid medium B), using a 10 mL syringe with an 18-gauge needle. The gel particles formed were stirred for approximately 10 minutes in liquid medium B. After this time, the particles were filtered to separate them from the liquid and were suspended in 24 mL of a 20 mM sodium citrate solution (liquid medium C).

### Example 31

100 mg of acetaminophen was suspended in 1.2 mL of canola oil with 1% v/v liquid soy lecithin. This suspension was mixed with 6 mL of 4 mL 2.5% w/v alginate solution and 2 mL 1.5% w/v starch. Once the mixture (liquid medium A) was homogenized, it was added dropwise from a height of 6-10 cm over 24 mL of a CaCl₂ solution at 3% w/v (liquid medium B), using a 10 mL syringe with an 18-gauge needle. The gel particles formed were stirred for approximately 10 minutes in liquid medium B. After this time, the particles were filtered to separate them from the liquid and were suspended in 24 mL of a 20 mM sodium citrate solution (liquid medium C).

### Example 32

100 mg of acetaminophen was suspended in 1.2 mL of water. This suspension was mixed with 6 mL of 4 mL 2.5% w/v alginate solution and 2 mL 1.5% w/v starch. Next, the mixture of oily phase and aqueous phase was emulsified by stirring, heating it to 40° C. The resulting emulsion (liquid medium A) was added dropwise from a height of 6 cm over 24 mL of a solution comprises of 4.8 mL of a 1.0% w/v chitosan solution and 19.2 mL of a 6% w/v CaCl₂ solution (liquid medium B) at room temperature, using a 10 mL syringe with an 18-gauge needle, always maintaining the temperature of liquid medium A at 40° C. throughout the drip. The gel particles formed were stirred for approximately 10 minutes in liquid medium B. After this time, the particles were filtered to separate them from the liquid and were suspended in 24 mL of a 20 mM sodium citrate solution (liquid medium C).

### Example 33

100 mg of acetaminophen was suspended in 1.2 mL of canola oil with 1% v/v liquid soy lecithin. This suspension was mixed with 6 mL of 4 mL 2.5% w/v alginate solution and 2 mL 1.5% w/v starch. Next, the mixture of oily phase and aqueous phase was emulsified by stirring, heating it to 40° C. The resulting emulsion (liquid medium A) was added dropwise from a height of 6 cm over 24 mL of a solution comprises of 4.8 mL of a 1.0% w/v chitosan solution and 19.2 mL of a 3% w/v CaCl₂ solution (liquid medium B) at room temperature, using a 10 mL syringe with an 18-gauge needle, always maintaining the temperature of liquid medium A at 40° C. throughout the drip. The gel particles formed were stirred for approximately 10 minutes in liquid medium B. After this time, the particles were filtered to separate them from the liquid and were suspended in 24 mL of a 20 mM sodium citrate solution (liquid medium C).

### Example 34

100 mg of acetaminophen was suspended in 1.2 mL of water. This suspension was mixed with 4.6 mL 2.5% w/v carrageenan. Once the mixture (liquid medium A) was homogenized, it was added dropwise from a height of 6-10 cm over 24 mL of a CaCl₂ solution at 3% w/v (liquid medium B), using a 10 mL syringe with an 18-gauge needle. The gel particles formed were stirred for approximately 10 minutes in liquid medium B. After this time, the particles were filtered to separate them from the liquid and were suspended in 24 mL of a 20 mM sodium citrate solution (liquid medium C).

### Example 35

100 mg of acetaminophen was suspended in 1.2 mL of canola oil with 1% v/v liquid soy lecithin. This suspension was mixed with 4.6 mL 2.5% w/v carrageenan. Once the mixture (liquid medium A) was homogenized, it was added dropwise from a height of 6-10 cm over 24 mL of a CaCl₂ solution at 3% w/v (liquid medium B), using a 10 mL syringe with an 18-gauge needle. The gel particles formed were stirred for approximately 10 minutes in liquid medium B. After this time, the particles were filtered to separate them from the liquid and were suspended in 24 mL of a 20 mM sodium citrate solution (liquid medium C).

The results and observations of these examples are shown in Table 2.

TABLE 2
Characterization Particles Containing Acetaminophen. Examples 1-35.

| | | | | | | | | Shape and | Color and |
|---|---|---|---|---|---|---|---|---|---|
| | | | | | chitosan | gauge | | size of gel | texture of |
| | gelling | water | Canola | CaCl₂ % | 1.0% | needle | Temp. | particles | the gel |
| Example | agent | mL | oil, mL | w/v | w/v, mL | (G) | ° C. | (mm ± 0.4) | particles |
| 1 | Alginate | 1.2 | — | 3 | — | 21 | 25 | Spheres | White, |
| | 2.5% w/v | | | | | | | (+/−) | soft |
| | | | | | | | | 2.04 mm, | |
| 2 | Alginate | — | 1.2 | 3 | — | 21 | 25 | Spheres | White, |
| | 2.5% w/v | | | | | | | (+/−) | less soft |
| | | | | | | | | 2.03 mm | |
| 3 | Alginate | 1.2 | — | 3 | — | 18 | 25 | Spheres | White, |
| | 2.5% w/v | | | | | | | (+/−) | soft |
| | | | | | | | | 2.32 mm, | |
| 4 | Alginate | — | 1.2 | 3 | — | 18 | 25 | Spheres | White, |
| | 2.5% w/v | | | | | | | (+/−) | less soft |
| | | | | | | | | 2.50 mm | |
| 5 | Alginate | 1.2 | — | 3 | 4.8 | 21 | 25 | Spheres | White, |
| | 2.5% w/v | | | | | | | (+/−) | less soft |
| | | | | | | | | 2.12 mm | |
| 6 | Alginate | 1.2 | — | 3 | 4.8 | 18 | 25 | Spheres | White, |
| | 2.5% w/v | | | | | | | (+/−) | less soft |
| | | | | | | | | 2.52 mm | |
| 7 | Alginate | — | 1.2 | 3 | 4.8 | 18 | 25 | Spheres | White, |
| | 2.5% w/v | | | | | | | (+/−) | less soft |
| | | | | | | | | 2.35 mm | |
| 8 | Gellan Gum | 1.2 | — | 3 | — | 18 | 40 | spheroids | Transparent, |
| | 2% w/v | | | | | | | amorphous | soft |
| | | | | | | | | (+/−) | |
| | | | | | | | | 3.04 mm | |
| 9 | Gellan Gum | 1.2 | — | 6 | — | 18 | 40 | Spheres | Transparent, |
| | 2% w/v | | | | | | | (+/−) | less soft |
| | | | | | | | | 2.74 mm | |
| 10 | Gellan Gum | — | 1.2 | 3 | — | 18 | 40 | Spheres | Transparent, |
| | 2% w/v | | | | | | | (+/−) | less soft |
| | | | | | | | | 2.69 mm | |
| 11 | Gellan Gum | 1.2 | — | 6 | — | 18 | 40 | spheroids | Transparent |
| | 1.5% w/v | | | | | | | amorphous | |
| 12 | Gellan Gum | — | 1.2 | 6 | — | 18 | 40 | spheroids | White |
| | 1.5% w/v | | | | | | | amorphous | |
| 13 | Gellan Gum | 1.2 | — | 6 | 4.8 | 18 | 40 | Spheres | White, |
| | 2% w/v | | | | | | | (+/−) | less soft |
| | | | | | | | | 2.89 mm | |
| 14 | Gellan Gum | — | 1.2 | 6 | — | 18 | 40 | spheroids | White |
| | 1.5% w/v | | | | | | | amorphous | |
| 15 | Pectin | 1.2 | — | 6 | — | 21 | 25 | spheres | spheres |
| | 5% w/v | | | | | | | were not | were not |
| | | | | | | | | formed | formed |
| 16 | Pectin | — | 1.2 | 6 | — | 21 | 25 | Lentil | spheres |
| | 5% w/v | | | | | | | shape | were not |
| | | | | | | | | | formed |
| 17 | Pectin | 1.2 | — | 6 | 4.8 | 21 | 25 | Spheres | transparent, |
| | 5% w/v | | | | | | | (+/−) | very soft |
| | | | | | | | | 1.98 mm | |
| 18 | 60/40 | 1.2 | — | 6 | | 18 | 40 | Spheres | transparent, |
| | Alginate | | | | | | | (+/−) | less soft |
| | 2.5% w/v)/ | | | | | | | 3.00 mm | |
| | Gellan Gum | | | | | | | | |
| | 2% p/v | | | | | | | | |
| 19 | Alginate | (24 mL) | — | 7.5 | 4.8 | 18 | 30 | hollow | Semi- |
| | 2.5% p w/v | | | | | | | spheres ≅ | transparent, |
| | | | | | | | | 4.0 mm | clump |
| | | | | | | | | | together |
| 20 | Alginate | (72 mL) | — | 7.5 | 4.8 | 18 | 30 | hollow | Transparent, |
| | 0.83% w/v | | | | | | | spheres ≅ | soft, stable |
| | | | | | | | | 4.0 mm | |
| 21 | Alginate | (72 mL) | — | 3 | 4.8 | 18 | 30 | hollow | Transparent, |
| | 0.83% w/v | | | | | | | spheres ≅ | soft, break |
| | | | | | | | | 4.0 mm | |
| 22 | Alginate | 1.2 | — | 3 | — | 27 | 25 | spheres | Transparent, |
| | 2.5% w/v | | | | | | | 0.3-0.5 | soft, stable |
| | | | | | | | | mm | |
| 23 | Alginate | — | 1.2 | 3 | — | 27 | 25 | spheres | White, |
| | 2.5% w/v | | | | | | | 0.3-0.5 | less soft |
| | | | | | | | | mm | |
| 24 | Alginate | 1.2 | — | 3 | 4.8 | 27 | 25 | spheres | White, |
| | 2.5% w/v | | | | | | | 0.3-0.5 | less soft |
| | | | | | | | | mm | |
| 25 | Alginate | —+ | 1.2 | 3 | 4.8 | 27 | 25 | spheres | White, |
| | 2.5% w/v | | | | | | | 0.3-0.5 | less soft |
| | | | | | | | | mm | |
| 26 | Alginate | 1.2 | — | 3 | — | 10 | 25 | spheres | Transparent, |
| | 2.5% w/v | | | | | | | 4.5-5 mm | soft, stable |
| 27 | Alginate | — | 1.2 | 3 | — | 10 | 25 | spheres | White, |
| | 2.5% w/v | | | | | | | 4.5-5 mm | less soft |
| 28 | Alginate | 1.2 | — | 3 | 4.8 | 10 | 25 | spheres | White, |
| | 2.5% w/v | | | | | | | 4.5-5 mm | less soft |
| 29 | Alginate | —+ | 1.2 | 3 | 4.8 | 10 | 25 | spheres | White, |
| | 2.5% w/v | | | | | | | 4.5-5 mm | less soft |
| 30 | 80/40 | 1.2 | — | 3 | — | 18 | 25 | Spheres | White, |
| | 2.5% w/v | | | | | | | (+/−) | soft |
| | alginate | | | | | | | 2.50 mm | |
| | solution/ | | | | | | | | |
| | 1.5% w/v | | | | | | | | |
| | starch | | | | | | | | |
| 31 | 80/40 | — | 1.2 | 3 | — | 18 | 25 | Spheres | White, |
| | 2.5% w/v | | | | | | | (+/−) | soft |
| | alginate | | | | | | | 2.50 mm | |
| | solution/ | | | | | | | | |
| | 1.5% w/v | | | | | | | | |
| | starch | | | | | | | | |
| 32 | 80/40 | 1.2 | — | 3 | 4.8 | 18 | 25 | Spheres | White, |
| | 2.5% w/v | | | | | | | (+/−) | soft |
| | alginate | | | | | | | 2.52 mm | |
| | solution/ | | | | | | | | |
| | 1.5% w/v | | | | | | | | |
| | starch | | | | | | | | |
| 33 | 80/40 | — | 1.2 | 3 | 4.8 | 18 | 25 | Spheres | White, |
| | 2.5% w/v | | | | | | | (+/−) | soft |
| | alginate | | | | | | | 2.35 mm | |
| | solution/ | | | | | | | | |
| | 1.5% w/v | | | | | | | | |
| | starch | | | | | | | | |
| 34 | Carrageenan | 1.2 | — | 3 | — | 18 | 25 | spheroids | White, |
| | 2.5% w/v | | | | | | | amorphous | soft |
| | | | | | | | | (+/−) | |
| | | | | | | | | 2.50 mm | |
| 35 | Carrageenan | — | 1.2 | 3 | — | 18 | 25 | spheroids | White, |
| | 2.5% w/v | | | | | | | amorphous | soft |
| | | | | | | | | (+/−) | |
| | | | | | | | | 2.50 mm | |

The Weibull maximum concentration in liquid (Fmax) and release rate of Higuchi models for acetaminophen, in liquid medium B and C for each of the above experiments is shown in Table 3.

TABLE 3
Acetaminophen Release for Examples 1-18 and 22-35.

| | Maximum | Release rate | Maximum | Release rate |
|---|---|---|---|---|
| | concentration | in liquid | concentration | in liquid |
| | in liquid B, | medium B, | in liquid C, | medium C, |
| Example | mg/mL | mg/mL/min | mg/mL | mg/mL/min |
| 1 | 56.8 | 22.63 | 13.43 | 2.12 |
| 3 | 52.5 | 20.37 | 10.9 | 9.7 |
| 4 | 64.3 | 11.5 | 12.4 | 1.47 |
| 5 | 64.4 | 15.26 | 21.8 | 3.33 |
| 6 | 77.7 | 15.82 | 23.4 | 3.37 |
| 7 | 76.0 | 15.95 | 22.7 | 3.11 |
| 8 | 90.6 | 24.47 | 19.5 | 9.6 |
| 9 | 76.7 | 22.60 | 21.0 | 5.0 |
| 10 | 76.2 | 22.58 | 27.0 | 4.1 |
| 12 | 103.2 | 23.07 | — | — |
| 15 | 118.9 | 21.48 | — | — |
| 16 | 59.8 | 21.81 | 37.0 | 2.8 |
| 17 | 75.0 | 25.06 | 22.1 | 1.60 |
| 18 | 65.0 | 1.87 | 15.6 | 2.8 |
| 22 | 91.5 | N.D. | 25.3 | 4.19 |
| 23 | 79.7 | N.D. | 9.82 | 1.69 |
| 24 | 42.9 | N.D. | 70.82 | 2.01 |
| 25 | 72.3 | N.D. | 100 | 2.30 |
| 26 | 62.6* | N.D. | 25.93 | 4.11 |
| 27 | 70.0* | N.D. | 25.14 | 4.22 |
| 28 | 63.30* | N.D. | 23.23 | 3.42 |
| 29 | 75.66* | N.D. | 21.89 | 3.82 |
| 30 | 93.74 | 15.05 | 10.76 | 1.84 |
| 31 | 95.31 | 15.24 | 10.10 | 1.78 |
| 32 | 77.97 | 12.20 | 9.43 | 1.67 |
| 33 | 95.32 | 15.24 | 11.99 | 1.79 |
| 34 | 91.30 | 13.71 | 19.5 | 2.97 |
| 35 | 83.45 | 12.72 | 14.21 | 2.50 |

(N.D.): not determined;
*at 10 min only, other samples were measured at 60 or 70 minutes.

B. Examples with Naproxen as API

In the next set of examples naproxen was used as API contained within the spheroid gel particles obtained according to the methods described herein.

### Example 36

110 mg of naproxen was suspended in 1.2 mL of water. This suspension was mixed with 4.8 mL of a 2.5% w/v alginate solution. Once the mixture (liquid medium A) was homogenized, it was added dropwise from a height of 6 cm over 24 mL of a 6% w/v CaCl₂ solution (liquid medium B), using a 10 mL syringe with a 21-gauge needle. The gel particles formed were stirred for approximately 10 min in liquid medium B. After this time, the particles were filtered to separate them from the liquid and were suspended in 24 mL of a 20 mM CaCl₂ solution (liquid medium C). Naproxen calcification (precipitation of naproxen calcium salts) was seen.

### Example 37

Naproxen 110 mg was suspended in 1.2 mL canola oil with 1% v/v liquid soy lecithin. This suspension was mixed with 4.8 mL of a 2.5% w/v alginate solution. The mixture of oily phase and aqueous phase was then emulsified by stirring. The resulting emulsion (liquid medium A) was added dropwise from a height of 6 cm over 24 mL of a 6% w/v CaCl₂ solution (liquid medium B), using a 10 mL syringe with a 21-gauge needle. The gel particles formed were stirred for approximately 10 min in liquid medium B. After this time, the particles were filtered to separate them from the liquid and were suspended in 24 mL of a 20 mM CaCl₂ solution (liquid medium C). Slight calcification of naproxen (precipitation of naproxen calcium salts) was seen.

### Example 38

110 mg of naproxen was suspended in 1.2 mL of water. This suspension was mixed with 4.8 mL of a 2.5% w/v alginate solution. Once the mixture (liquid medium A) was homogenized, it was added dropwise from a height of 6 cm over 24 mL of a solution comprises of 4.8 mL of a 1.0% w/v chitosan solution and 19.2 mL of a 6% w/v CaCl₂ solution (liquid medium B), using a 10 mL syringe with an 18-gauge needle. The gel particles formed were stirred for approximately 10 min in liquid medium B. After this time, the particles were filtered to separate them from the liquid and were suspended in 24 mL of a 20 mM CaCl₂ solution (liquid medium C). Naproxen calcification (precipitation of naproxen calcium salts) was seen.

### Example 39

Naproxen 110 mg was suspended in 1.2 mL canola oil with 1% v/v liquid soy lecithin. This suspension was mixed with 4.8 mL of a 2.5% w/v alginate solution. The mixture of oily phase and aqueous phase was then emulsified by stirring. The resulting emulsion (liquid medium A) was added dropwise from a height of 6 cm over 24 mL of a solution comprises of 4.8 mL of a 1.0% w/v chitosan solution and 19.2 mL of a 6% w/v CaCl₂ solution (liquid medium B), using a 10 mL syringe with an 18-gauge needle. The gel particles formed were stirred for approximately 10 min in liquid medium B. After this time, the particles were filtered to separate them from the liquid and were suspended in 24 mL of a 20 mM CaCl₂ solution (liquid medium C). Slight calcification of naproxen (precipitation of naproxen calcium salts) was seen.

### Example 40

110 mg of naproxen was suspended in 1.2 mL of water. This suspension was mixed with 4.8 mL of a 2.5% w/v alginate solution. Once the mixture (liquid medium A) was homogenized, it was added dropwise from a height of 6 cm over 24 mL of a CaCl₂ solution at 3% w/v (liquid medium B), using a 10 mL syringe with an 18-gauge needle. The gel particles formed were stirred for approximately 10 min in liquid medium B. After this time, the particles were filtered to separate them from the liquid and were suspended in 24 mL of a 20 mM CaCl₂ solution (liquid medium C).

### Example 41

Naproxen 110 mg was suspended in 1.2 mL canola oil with 1% v/v liquid soy lecithin. This suspension was mixed with 4.8 mL of a 2.5% w/v alginate solution. The mixture of oily phase and aqueous phase was then emulsified by stirring. The resulting emulsion (liquid medium A) was added dropwise from a height of 6 cm over 24 mL of a 3% w/v CaCl₂ solution (liquid medium B), using a 10 mL syringe with an 18-gauge needle. The gel particles formed were stirred for approximately 10 minutes in liquid medium B. After this time, the particles were filtered to separate them from the liquid and were suspended in 24 mL of a 20 mM CaCl₂ solution (liquid medium C). Slight calcification of naproxen (precipitation of naproxen calcium salts) was seen.

### Example 42

110 mg of naproxen was suspended in 1.2 mL of water. This suspension was mixed with 4.8 mL of a 2.5% w/v alginate solution. Once the mixture (liquid medium A) was homogenized, it was added dropwise from a height of 6 cm over 24 mL of a solution comprises of 4.8 mL of a 1.0% w/v chitosan solution and 19.2 mL of a 3% w/v CaCl₂ solution (liquid medium B), using a 10 mL syringe with a 21-gauge needle. The gel particles formed were stirred for approximately 10 minutes in liquid medium B. After this time, the particles were filtered to separate them from the liquid and were suspended in 24 mL of a 20 mM CaCl₂ solution (liquid medium C).

### Example 43

Naproxen 110 mg was suspended in 1.2 mL canola oil with 1% v/v liquid soy lecithin. This suspension was mixed with 4.8 mL of a 2.5% w/v alginate solution. The mixture of oily phase and aqueous phase was then emulsified by stirring. The resulting emulsion (liquid medium A) was added dropwise from a height of 6 cm over 24 mL of a solution comprises of 4.8 mL of a 1.0% w/v chitosan solution and 19.2 mL of a CaCl₂ solution at 3% w/v (liquid medium B), using a 10 mL syringe with a 21-gauge needle. The gel particles formed were stirred for approximately 10 minutes in liquid medium B. After this time, the particles were filtered to separate them from the liquid and were suspended in 24 mL of a 20 mM CaCl₂ solution (liquid medium C).

### Example 44

110 mg of naproxen was suspended in 1.2 mL of water. This suspension was mixed with 4.8 mL of a 2% w/v gellan gum solution. Once the mixture (liquid medium A) was homogenized, it was heated to 40° C. and was added dropwise from a height of 6 cm, over 24 mL of a 6% w/v CaCl₂ solution (liquid medium B), at room temperature, using a 10 mL syringe with an 18-gauge needle, always maintaining the temperature of liquid medium A at 40° C. throughout the drip. The gel particles formed were stirred for approximately 10 minutes in liquid medium B. After this time, the particles were filtered to separate them from the liquid and were suspended in 24 mL of a 20 mM CaCl₂ solution (liquid medium C). Naproxen calcification (precipitation of naproxen calcium salts) was seen.

### Example 45

Naproxen 110 mg was suspended in 1.2 mL canola oil with 1% v/v liquid soy lecithin. This suspension was mixed with 4.8 mL of a 2% w/v gellan gum solution. Next, the mixture of oily phase and aqueous phase was emulsified by stirring, heating it to 40° C. The resulting emulsion (liquid medium A) was added dropwise from a height of 6 cm over 24 mL of a 6% w/v CaCl₂ solution (liquid medium B) at room temperature, using a 10 mL syringe with an 18-gauge needle, always maintaining the temperature of the liquid medium A at 40° C. throughout the dripping. The gel particles formed were stirred for approximately 10 min in liquid medium B. After this time, the particles were filtered to separate them from the liquid and were suspended in 24 mL of a 20 mM CaCl₂ solution (liquid medium C). Slight calcification of naproxen (precipitation of naproxen calcium salts) was seen.

### Example 46

110 mg of naproxen was suspended in 1.2 mL of water. This suspension was mixed with 4.8 mL of a 2% w/v gellan gum solution. Once the mixture (liquid medium A) has been homogenized, it was heated to 40° C. and 24 mL of a solution comprises of 4.8 mL of a 1.0% chitosan solution was added dropwise from a height of 6 cm over 24 mL of a 6% w/v CaCl₂ solution (liquid medium B) at room temperature, using a 10 mL syringe with an 18-gauge needle, always maintaining the temperature of liquid medium A at 40° C. throughout the drip. The gel particles formed were stirred for approximately 10 minutes in liquid medium B. After this time, the particles were filtered to separate them from the liquid and were suspended in 24 mL of a 20 mM CaCl₂ solution (liquid medium C). Naproxen calcification (precipitation of naproxen calcium salts) was seen.

### Example 47

Naproxen 110 mg was suspended in 1.2 mL canola oil with 1% v/v liquid soy lecithin. This suspension was mixed with 4.8 mL of a 2% w/v gellan gum solution. Next, the mixture of oily phase and aqueous phase was emulsified by stirring, heating it to 40° C. The resulting emulsion (liquid medium A) was added dropwise from a height of 6 cm over 24 mL of a solution comprises of 4.8 mL of a 1.0% w/v chitosan solution and 19.2 mL of a 6% w/v CaCl₂ solution (liquid medium B) at room temperature, using a 10 mL syringe with an 18-gauge needle, always maintaining the temperature of liquid medium A at 40° C. throughout the drip. The gel particles formed were stirred for approximately 10 minutes in liquid medium B. After this time, the particles were filtered to separate them from the liquid and were suspended in 24 mL of a 20 mM CaCl₂ solution (liquid medium C). Slight calcification of naproxen (precipitation of naproxen calcium salts) was seen.

### Example 48

110 mg of naproxen was suspended in 1.2 mL of water. This suspension was mixed with 4.8 mL of a 2% w/v gellan gum solution. Once the mixture (liquid medium A) was homogenized, it was heated to 40° C. was added dropwise from a height of 6 cm, over 24 mL of a 3% w/v CaCl₂ solution (liquid medium B) at room temperature, using a 10 mL syringe with an 18-gauge needle, always maintaining the temperature of liquid medium A at 40° C. throughout the drip. The gel particles formed were stirred for approximately 10 minutes in liquid medium B. After this time, the particles were filtered to separate them from the liquid and were suspended in 24 mL of a 20 mM CaCl₂ solution (liquid medium C).

### Example 49

Naproxen 110 mg was suspended in 1.2 mL canola oil with 1% v/v liquid soy lecithin. This suspension was mixed with 4.8 mL of a 2% w/v gellan gum solution. Next, the mixture of oily phase and aqueous phase was emulsified by stirring, heating it to 40° C. The resulting emulsion (liquid medium A) was added dropwise from a height of 6 cm over 24 mL of a 3% w/v CaCl₂ solution (liquid medium B) at room temperature, using a 10 mL syringe with an 18-gauge needle, always maintaining the temperature of the liquid medium A at 40° C. throughout the dripping. The gel particles formed were stirred for approximately 10 minutes in liquid medium B. After this time, the particles were filtered to separate them from the liquid and were suspended in 24 mL of a 20 mM CaCl₂ solution (liquid medium C).

### Example 50

110 mg of naproxen was suspended in 1.2 mL of water. This suspension was mixed with 4.8 mL of a 2% w/v gellan gum solution. Once the mixture (liquid medium A) has been homogenized, it was heated to 40° C. was added dropwise from a height of 6 cm, over 24 mL of a 3% w/v CaCl₂ solution (liquid medium B) at room temperature, using a 10 mL syringe with an 18-gauge needle, always maintaining the temperature of liquid medium A at 40° C. throughout the drip. The gel particles formed were stirred for approximately 10 minutes in liquid medium B. After this time, the particles were filtered to separate them from the liquid and were suspended in 24 mL of a 20 mM CaCl₂ solution (liquid medium C).

### Example 51

Naproxen 110 mg was suspended in 1.2 mL canola oil with 1% v/v liquid soy lecithin. This suspension was mixed with 4.8 mL of a 2% w/v gellan gum solution. Next, the mixture of oily phase and aqueous phase was emulsified by stirring, heating it to 40° C. The resulting emulsion (liquid medium A) was added dropwise from a height of 6 cm over 24 mL of a solution comprises of 4.8 mL of a 1.0% w/v chitosan solution and 19.2 mL of a 3% w/v CaCl₂ solution (liquid medium B) at room temperature, using a 10 mL syringe with an 18-gauge needle, always maintaining the temperature of liquid medium A at 40° C. throughout the drip. The gel particles formed were stirred for approximately 10 minutes in liquid medium B. After this time, the particles were filtered to separate them from the liquid and were suspended in 24 mL of a 20 mM CaCl₂ solution (liquid medium C). Slight calcification of naproxen (precipitation of naproxen calcium salt) was seen.

### Example 52

110 mg of naproxen was suspended in 1.2 mL of water. This suspension was mixed with 4.8 mL of a 5% w/v pectin solution. Once the mixture (liquid medium A) has been homogenized, it was heated to 40° C. was added dropwise from a height of 6 cm, over 24 mL of a 6% w/v CaCl₂ solution (liquid medium B) at room temperature, using a 10 mL syringe with a 21-gauge needle, always maintaining the temperature of liquid medium A at 40° C. throughout the drip. The gel particles formed were stirred for approximately 10 minutes in liquid medium B. After this time, the particles were filtered to separate them from the liquid and were suspended in 24 mL of a 20 mM CaCl₂ solution (liquid medium C).

### Example 53

110 mg of naproxen was suspended in 1.2 mL of water. This suspension was mixed with 4.8 mL of a solution comprises of 60% of a 2.5% w/v alginate solution and 40% of a 2% w/v gellan gum solution. Once the mixture (liquid medium A) was homogenized, it was heated to 40° was added dropwise from a height of 6 cm over 24 mL of a 6% w/v CaCl₂ solution (liquid medium B) to room temperature, using a 10 mL syringe with an 18-gauge needle, always maintaining the temperature of liquid medium A at 40° C. throughout the drip. The gel particles formed were stirred for approximately 10 minutes in liquid medium B. After this time, the particles were filtered to separate them from the liquid and were suspended in 24 mL of a 20 mM CaCl₂ solution (liquid medium C).

### Example 54

Naproxen 110 mg was suspended in 1.2 mL canola oil with 1% v/v liquid soy lecithin. This suspension was mixed with 4.8 mL of a solution comprises of 60% of a 2.5% w/v alginate solution and 40% of a 2% w/v gellan gum solution. Next, the mixture of oily phase and aqueous phase was emulsified by stirring, heating it to 40° C. The resulting emulsion (liquid medium A) was added dropwise from a height of 6 cm over 24 mL of a 3% w/v CaCl₂ solution (liquid medium B) at room temperature, using a 10 mL syringe with an 18-gauge needle, always maintaining the temperature of the liquid medium A at 40° C. throughout the dripping. The gel particles formed were stirred for approximately 10 minutes in liquid medium B. After this time, the particles were filtered to separate them from the liquid and were suspended in 24 mL of a 20 mM CaCl₂ solution (liquid medium C).

### Example 55

110 mg of naproxen was suspended in 1.2 mL of a 7.5% w/v CaCl₂ solution. This suspension was mixed with 4.8 mL of a 1.0% w/v chitosan solution. Once the mixture (liquid medium A) was homogenized, it was added dropwise from a height of 6 cm over 72 mL of a 0.83% w/v alginate solution (liquid medium B), using a 10 mL syringe mL with an 18-gauge needle. The gel particles formed were stirred for approximately 7 minutes in liquid medium B. After this time, the particles were filtered to separate them from the liquid and were suspended in 24 mL of a 20 mM CaCl₂ solution (liquid medium C).

### Example 56

110 mg of naproxen was suspended in 1.2 mL of water. This suspension was mixed with 4.8 mL of a 1.5% w/v alginate solution. Once the mixture (liquid medium A) was homogenized, it was added dropwise from a height of 6 cm over 24 mL of a 3% w/v CaCl₂ solution (liquid medium B), using a 10 mL syringe with an 18-gauge needle. The gel particles formed were stirred for approximately 10 min in liquid medium B. After this time, the particles were filtered to separate them from the liquid and were suspended in 24 mL of a 20 mM CaCl₂ solution (liquid medium C).

### Example 57

Naproxen 110 mg was suspended in 1.2 mL canola oil with 1% v/v liquid soy lecithin. This suspension was mixed with 4.8 mL of a 1.5% w/v alginate solution. Once the mixture (liquid medium A) was homogenized, it was added dropwise from a height of 6 cm over 24 mL of a 3% w/v CaCl₂ solution (liquid medium B), using a 10 mL syringe with an 18-gauge needle. The gel particles formed were stirred for approximately 10 min in liquid medium B. After this time, the particles were filtered to separate them from the liquid and were suspended in 24 mL of a 20 mM CaCl₂ solution (liquid medium C).

### Example 58

110 mg of naproxen was suspended in 1.2 mL of water. This suspension was mixed with 4.8 mL of a 1.5% w/v alginate solution. Once the mixture (liquid medium A) was homogenized, it was added dropwise from a height of 6 cm over 24 mL of a solution comprises of 4.8 mL of a 1.0% w/v chitosan solution and 19.2 mL of a 3% w/v CaCl₂ solution (liquid medium B), using a 10 mL syringe with a 21-gauge needle. The gel particles formed were stirred for approximately 10 minutes in liquid medium B. After this time, the particles were filtered to separate them from the liquid and were suspended in 24 mL of a 20 mM CaCl₂ solution (liquid medium C).

### Example 59

Naproxen 110 mg was suspended in 1.2 mL canola oil with 1% v/v liquid soy lecithin. This suspension was mixed with 4.8 mL of a 1.5% w/v alginate solution. The mixture of oily phase and aqueous phase was then emulsified by stirring. The resulting emulsion (liquid medium A) was added dropwise from a height of 6 cm over 24 mL of a solution comprises of 4.8 mL of a 1.0% w/v chitosan solution and 19.2 mL of a CaCl₂ solution at 3% w/v (liquid medium B), using a 10 mL syringe with a 21-gauge needle. The gel particles formed were stirred for approximately 10 minutes in liquid medium B. After this time, the particles were filtered to separate them from the liquid and were suspended in 24 mL of a 20 mM CaCl₂ solution (liquid medium C).

The results and observations of these examples are shown in Table 4.

TABLE 4
Characterization Particles Containing Naproxen. Examples 36-59.

| | | | | | | | | Shape and | |
|---|---|---|---|---|---|---|---|---|---|
| | | | | | | | | size of gel | |
| | | | | | | | | particles | |
| | | | | | | | | (mm ± 0.5), | |
| | | | | | | | | colour and | |
| | | | | | chitosan | gauge | | texture of | |
| | gelling | water | Canola | CaCl₂ % | 1.0% | needle | Temp. | the gel | |
| Example | agent | mL | oil, mL | w/v | w/v, mL | (G) | ° C. | particles | comment |
| 36 | Alginate | 1.2 | — | 6 | — | 21 | 25 | spherical or | Naproxen |
| | 2.5% w/v | | | | | | | spheroidal | calcification |
| | | | | | | | | particles | was seen |
| | | | | | | | | 2.15 mm | |
| | | | | | | | | white, | |
| | | | | | | | | very soft | |
| 37 | Alginate | — | 1.2 | 6 | — | 21 | 25 | spherical or | mild |
| | 2.5% w/v | | | | | | | spheroidal | naproxen |
| | | | | | | | | particles | calcification |
| | | | | | | | | 2.14 mm, | was |
| | | | | | | | | white, | observed |
| | | | | | | | | less soft | |
| 38 | Alginate | 1.2 | — | 6 | 4.8 | 18 | 25 | spherical or | Naproxen |
| | 2.5% w/v | | | | | | | spheroidal | calcification |
| | | | | | | | | particles | was |
| | | | | | | | | (+/−) | observed |
| | | | | | | | | 2.6 mm, | |
| | | | | | | | | white, | |
| | | | | | | | | very soft | |
| 39 | Alginate | — | 1.2 | 6 | 4.8 | 18 | 25 | spherical or | mild |
| | 2.5% w/v | | | | | | | spheroidal | naproxen |
| | | | | | | | | particles | calcification |
| | | | | | | | | (+/−) | was |
| | | | | | | | | 2.6 mm, | observed |
| | | | | | | | | white, | |
| | | | | | | | | less soft | |
| 40 | Alginate | 1.2 | — | 3 | — | 21 | 25 | spherical or | — |
| | 2.5% w/v | | | | | | | spheroidal | |
| | | | | | | | | particles | |
| | | | | | | | | (+/−) | |
| | | | | | | | | 2.05 mm, | |
| | | | | | | | | white, soft | |
| 41 | Alginate | — | 1.2 | 3 | — | 21 | 25 | spherical or | — |
| | 2.5% w/v | | | | | | | spheroidal | |
| | | | | | | | | particles | |
| | | | | | | | | (+/−) | |
| | | | | | | | | 2.1 mm, | |
| | | | | | | | | white, | |
| | | | | | | | | very soft | |
| 42 | Alginate | 1.2 | — | 3 | 4.8 | 21 | 25 | spherical or | — |
| | 2.5% w/v | | | | | | | spheroidal | |
| | | | | | | | | particles | |
| | | | | | | | | (+/−) | |
| | | | | | | | | 2.2 mm, | |
| | | | | | | | | white, | |
| | | | | | | | | very soft | |
| 43 | Alginate | — | 1.2 | 3 | 4.8 | 21 | 25 | spherical or | — |
| | 2.5% w/v | | | | | | | spheroidal | |
| | | | | | | | | particles | |
| | | | | | | | | (+/−) | |
| | | | | | | | | 2.1 mm, | |
| | | | | | | | | white, | |
| | | | | | | | | very soft | |
| 44 | Gellan | 1.2 | — | 6 | — | 18 | 40 | spherical or | Naproxen |
| | Gum | | | | | | | spheroidal | calcification |
| | 2% w/v | | | | | | | particles | was |
| | | | | | | | | (+/−) | observed |
| | | | | | | | | 2.9 mm, | |
| | | | | | | | | white, soft | |
| 45 | Gellan | — | 1.2 | 6 | — | 18 | 40 | spherical or | mild |
| | Gum | | | | | | | spheroidal | naproxen |
| | 2% w/v | | | | | | | particles | calcification |
| | | | | | | | | (+/−) | was |
| | | | | | | | | 2.8 mm, | observed |
| | | | | | | | | white | |
| | | | | | | | | less soft | |
| 46 | Gellan | 1.2 | — | 6 | 4.8 | 18 | 40 | spherical or | Naproxen |
| | Gum | | | | | | | spheroidal | calcification |
| | 2% w/v | | | | | | | particles | was |
| | | | | | | | | (+/−) | observed |
| | | | | | | | | 2.7 mm, | |
| | | | | | | | | white | |
| | | | | | | | | very soft | |
| 47 | Gellan | — | 1.2 | 6 | 4.8 | 18 | 40 | spherical or | mild |
| | Gum | | | | | | | spheroidal | naproxen |
| | 2% w/v | | | | | | | particles | calcification |
| | | | | | | | | (+/−) | was |
| | | | | | | | | 2.5 mm, | observed |
| | | | | | | | | white, | |
| | | | | | | | | less soft | |
| 48 | Gellan | 1.2 | — | 3 | — | 18 | 40 | spherical or | — |
| | Gum | | | | | | | spheroidal | |
| | 2% w/v | | | | | | | particles | |
| | | | | | | | | (+/−) 2.3- | |
| | | | | | | | | 2.8 mm, | |
| | | | | | | | | transparent, | |
| | | | | | | | | soft | |
| 49 | Gellan | — | 1.2 | 3 | — | 18 | 40 | spherical or | — |
| | Gum | | | | | | | spheroidal | |
| | 2% w/v | | | | | | | particles | |
| | | | | | | | | 2-2.5 mm, | |
| | | | | | | | | transparent, | |
| | | | | | | | | less soft | |
| 50 | Gellan | 1.2 | — | 3 | 4.8 | 18 | 40 | spherical or | naproxen |
| | Gum | | | | | | | spheroidal | calcification |
| | 2% w/v | | | | | | | particles | was |
| | | | | | | | | 2.1-2.8 mm, | observed |
| | | | | | | | | transparent, | |
| | | | | | | | | soft | |
| 51 | Gellan | — | 1.2 | 3 | 4.8 | 18 | 40 | spherical or | — |
| | Gum | | | | | | | spheroidal | |
| | 2% w/v | | | | | | | particles | |
| | | | | | | | | (+/−) | |
| | | | | | | | | 2.7 mm, | |
| | | | | | | | | transparent, | |
| | | | | | | | | very soft | |
| 52 | Pectin | 1.2 | — | 3 | — | 21 | 25 | −−− | No |
| | 2.5% w/v | | | | | | | | spherical- |
| | | | | | | | | | and/or |
| | | | | | | | | | spheroidal- |
| | | | | | | | | | shaped |
| | | | | | | | | | particles |
| | | | | | | | | | were |
| | | | | | | | | | formed |
| 53 | 60/40 | 1.2 | — | 6 | — | 18 | 40 | spherical or | naproxen |
| | (Alginate | | | | | | | spheroidal | calcification |
| | 2.5% w/v)/ | | | | | | | particles | was |
| | (Gellan | | | | | | | (+/−) | observed |
| | Gum | | | | | | | 2.80 mm | |
| | 2% w/v) | | | | | | | | |
| 54 | 60/40 | — | 1.2 | 3 | — | 18 | 40 | spherical or | semi- |
| | (Alginate | | | | | | | spheroidal | transparent, |
| | 2.5% w/v)/ | | | | | | | particles | less soft |
| | (Gellan | | | | | | | (+/−) | |
| | Gum 2% | | | | | | | 2.75 mm, | |
| | w/v) | | | | | | | | |
| 55 | Alginate | (72 mL) | — | 7.5 | 4.8 | 18 | 30 | Hollow | naproxen |
| | 0.83% w/v | | | | | | | spherical or | calcification, |
| | | | | | | | | spheroidal | break |
| | | | | | | | | particles ≅ | |
| | | | | | | | | 4.0 mm | |
| 56 | Alginate | 1.2 | — | 3 | — | 18 | 25 | spherical or | |
| | 1.5% w/v | | | | | | | spheroidal | |
| | | | | | | | | particles ≅ | |
| | | | | | | | | 2.1 mm | |
| | | | | | | | | white, | |
| | | | | | | | | very soft | |
| 57 | Alginate | — | 1.2 | 3 | — | 18 | 25 | spherical or | |
| | 1.5% w/v | | | | | | | spheroidal | |
| | | | | | | | | particles ≅ | |
| | | | | | | | | 2.1 mm | |
| | | | | | | | | white, | |
| | | | | | | | | very soft | |
| 58 | Alginate | 1.2 | — | 3 | 4.8 | 18 | 25 | spherical or | |
| | 1.5% w/v | | | | | | | spheroidal | |
| | | | | | | | | particles ≅ | |
| | | | | | | | | 2.1 mm | |
| | | | | | | | | white, | |
| | | | | | | | | very soft | |
| 59 | Alginate | — | 1.2 | 3 | 4.8 | 18 | 25 | spherical or | |
| | 1.5% w/v | | | | | | | spheroidal | |
| | | | | | | | | particles ≅ | |
| | | | | | | | | 2.1 mm | |
| | | | | | | | | white, | |
| | | | | | | | | very soft | |

The Weibull maximum concentration in liquid (Fmax) and release rate of Higuchi models for naproxen, in liquid medium B and C for each of the above experiments is shown in Table 5.

TABLE 5
Naproxen Release for Examples 36-44, 47-50, and 53-59.

| | Maximum | Release rate | Maximum | Release rate |
|---|---|---|---|---|
| | concentration | in liquid | concentration | in liquid |
| | in liquid B, | medium B, | in liquid C, | medium C, |
| Example | mg/mL | mg/mL/min | mg/mL | mg/mL/min |
| 36 | 9.3 | 1.32 | 14.0 | 1.00 |
| 37 | 7.3 | 1.06 | 7.8 | 0.2 |
| 38 | 13.0 | 0.70 | 4.7 | 0.10 |
| 39 | 13.3 | 1.10 | 9.5 | 0.28 |
| 40 | 52.0 | 1.43 | 6.9 | 0.10 |
| 41 | 41.0 | 2.30 | 6.6 | 0.10 |
| 42 | 2.9 | 0.50 | 3.2 | 0.20 |
| 43 | 8.8 | 0.60 | 2.4 | 0.30 |
| 44 | 42.5 | 4.50 | 9.4 | 0.20 |
| 47 | 6.2 | 1.13 | 7.0 | 0.20 |
| 48 | 42.0 | 1.48 | 8.9 | 0.90 |
| 49 | 19.5 | 1.13 | 6.4 | 0.50 |
| 50 | 108.0 | 2.26 | N.D. | N.D. |
| 53 | 31.5 | 3.00 | 36.0 | 1.30 |
| 54 | 22.5 | 1.40 | 4.0 | 0.20 |
| 55 | 141.0 | 1.10 | 35.6 | 0.80 |
| 56 | 81.0 | N.D. | 28.0 | 4.34 |
| 57 | 66.5 | N.D. | 35.7 | 4.20 |
| 58 | 5.1 | N.D. | 18.5 | 3.16 |
| 59 | 4.3 | N.D. | 24.6 | 4.00 |

(N.D.): Not determined.
For all samples were measured at 60 or 70 minutes.

C. Examples with Mefenamic Acid as API

In the next set of examples, mefenamic acid was used as API contained within the spherical gel particles, obtained according to the methods described herein.

### Example 60

100 mg of mefenamic acid was suspended in 1.2 mL of water. This suspension was mixed with 4.8 mL of a 2% w/v alginate solution. Once the mixture (liquid medium A) was homogenized, it was added dropwise from a height of 6 cm over 24 mL of a CaCl₂ solution at 3% w/v (liquid medium B), using a 10 mL syringe with an 18-gauge needle. The gel particles formed were stirred for approximately 10 minutes in liquid medium B. After this time, the particles were filtered to separate them from the liquid and were suspended in 24 mL of a 20 mM CaCl₂ solution (liquid medium C).

### Example 61

100 mg of mefenamic acid was suspended in 1.2 mL of canola oil with 1% v/v liquid soy lecithin. This suspension was mixed with 4.8 mL of a 2% w/v alginate solution. The mixture of oily phase and aqueous phase was then emulsified by stirring. The resulting emulsion (liquid medium A) was added dropwise from a height of 6 cm over 24 mL of a 3% w/v CaCl₂ solution (liquid medium B), using a 10 mL syringe with an 18-gauge needle. The gel particles formed were stirred for approximately 10 minutes in liquid medium B. After this time, the particles were filtered to separate them from the liquid and were suspended in 24 mL of a 20 mM CaCl₂) solution (liquid medium C).

### Example 62

100 mg of mefenamic acid was suspended in 1.2 mL of water. This suspension was mixed with 4.8 mL of a 1.5% w/v alginate solution. Once the mixture (liquid medium A) was homogenized, it was added dropwise from a height of 6 cm over 24 mL of a solution comprises of 4.8 mL of a 1.0% w/v chitosan solution and 19.2 mL of a 7.5% w/v CaCl₂ solution (liquid medium B), using a 10 mL syringe with a 21-gauge needle. The gel particles formed were stirred for approximately 10 minutes in liquid medium B. After this time, the particles were filtered to separate them from the liquid and were suspended in 24 mL of a 20 mM CaCl₂ solution (liquid medium C).

### Example 63

100 mg of mefenamic acid was suspended in 1.2 mL of a 3% w/v CaCl₂) solution. This suspension was mixed with 4.8 mL of a 1.0% w/v chitosan solution. Once the mixture (liquid medium A) was homogenized, it was added dropwise from a height of 6 cm over 72 mL of a 0.83% w/v alginate solution (liquid medium B), using a 10 mL syringe with a 21-gauge needle. The gel particles formed were stirred for approximately 7 minutes in liquid medium B. After this time, the particles were filtered to separate them from the liquid and were suspended in 24 mL of a 20 mM CaCl₂ solution (liquid medium C).

### Example 64

100 mg of mefenamic acid was suspended in 1.2 mL of water. This suspension was mixed with 4.8 mL of a 1.5% w/v alginate solution. Once the mixture (liquid medium A) was homogenized, it was added dropwise from a height of 6 cm over 24 mL of a solution composed of 4.8 mL of a 1.0% w/v chitosan solution and 19.2 mL of a 7.5% w/v CaCl₂ solution (liquid medium B), using a 10 mL syringe with a 21-gauge needle. The gel particles formed were stirred for approximately 10 minutes in liquid medium B. After this time, the particles were filtered to separate them from the liquid and were suspended in 24 mL of a 20 mM CaCl₂ solution (liquid medium C).

### Example 65

100 mg of mefenamic acid was suspended in 1.2 mL of canola oil with 1% v/v liquid soy lecithin. This suspension was mixed with 4.8 mL of a 1.5% w/v alginate solution. Once the mixture (liquid medium A) was homogenized, it was added dropwise from a height of 6 cm over 24 mL of a solution composed of 4.8 mL of a 1.0% w/v chitosan solution and 19.2 mL of a 7.5% w/v CaCl₂ solution (liquid medium B), using a 10 mL syringe with a 21-gauge needle. The gel particles formed were stirred for approximately 10 minutes in liquid medium B. After this time, the particles were filtered to separate them from the liquid and were suspended in 24 mL of a 20 mM CaCl₂ solution (liquid medium C).

### Example 66

100 mg of mefenamic acid was suspended in 1.2 mL of a 3% w/v CaCl₂) solution. This suspension was mixed with 4.8 mL of a 1.0% w/v chitosan solution. Once the mixture (liquid medium A) was homogenized, it was added dropwise from a height of 6 cm over 72 mL of a 0.83% w/v alginate solution (liquid medium B), using a 10 mL syringe with a 21-gauge needle. The gel particles formed were stirred for approximately 7 minutes in liquid medium B. After this time, the particles were filtered to separate them from the liquid and were suspended in 24 mL of a 20 mM CaCl₂ solution (liquid medium C).

The results and observations of these examples are shown in Table 6.

TABLE 6
Characterization Particles Containing Mefenamic Acid. Examples 60-66.

| | | | | | | | | Shape and | Color and |
|---|---|---|---|---|---|---|---|---|---|
| | | | | | chitosan | gauge | | size of gel | texture of |
| | gelling | water | Canola | CaCl₂ % | 1.0% | needle | Temp. | particles | the gel |
| Example | agent | mL | oil, mL | w/v | w/v, mL | (G) | ° C. | (mm ± 0.4) | particles |
| 60 | Alginate | 1.2 | — | 3 | — | 18 | 25 | Spheroidal or | Transparent, |
| | 2.0% w/v | | | | | | | spheroidal | soft, stable |
| | | | | | | | | particles | |
| | | | | | | | | (+/−) | |
| | | | | | | | | 2.70 mm | |
| 61 | Alginate | — | 1.2 | 3 | — | 18 | 25 | Spheroidal or | Transparent, |
| | 2.0% w/v | | | | | | | spheroidal | soft, stable |
| | | | | | | | | particles | |
| | | | | | | | | (+/−) | |
| | | | | | | | | 2.65 mm | |
| 62 | Alginate | 1.2 | — | 3 | 4.8 | 18 | 25 | Spheroidal or | Transparent, |
| | 1.5% w/v | | | | | | | spheroidal | soft, stable |
| | | | | | | | | particles | |
| | | | | | | | | (+/−) | |
| | | | | | | | | 2.10 mm | |
| 63 | Alginate | (72 mL) | | 7.5 | 4.8 | 18 | 30 | Hollow | Transparent, |
| | 0.83% w/v | | | | | | | spherical or | soft, stable |
| | | | | | | | | spheroidal | |
| | | | | | | | | particles, ≅4.0 mm | |
| 64 | Alginate | 1.2 | — | 3 | 4.8 | 21 | 25 | Spheroidal or | Transparent, |
| | 1.5% w/v | | | | | | | spheroidal | soft, stable |
| | | | | | | | | particles | |
| | | | | | | | | (+/−) | |
| | | | | | | | | 2.10 mm | |
| 65 | Alginate | — | 1.2 | 3 | 4.8 | 21 | 25 | Spheroidal or | Transparent, |
| | 1.5% w/v | | | | | | | spheroidal | soft, stable |
| | | | | | | | | particles | |
| | | | | | | | | (+/−) | |
| | | | | | | | | 2.10 mm | |
| 66 | Alginate | (72 mL) | | 7.5 | 4.8 | 21 | 30 | Hollow | Transparent, |
| | 0.83% w/v | | | | | | | spherical or | soft, stable |
| | | | | | | | | spheroidal | |
| | | | | | | | | particles, ≅4.0 mm | |

No release of mefenamic acid into the aqueous medium such as liquid medium B as liquid medium C was observed, because all the content was in the matrix of the sphere. Therefore, the Weibull maximum concentration in liquid (Fmax) and release rate of Higuchi models for naproxen were not possible to obtain.

D. Examples with Chlorphenamine Maleate as API

In the next set of examples, chlorphenamine maleate was used as an API contained within the spheroid gel particles obtained according to the methods described herein.

### Example 67

40 mg of chlorphenamine maleate was suspended in 1.2 mL of water. This suspension was mixed with 4.8 mL of a 2% w/v alginate solution. Once the mixture (liquid medium A) was homogenized, it was added dropwise from a height of 6 cm over 24 mL of a CaCl₂ solution at 3% w/v (liquid medium B), using a 10 mL syringe with an 18-gauge needle. The gel particles formed were stirred for approximately 10 minutes in liquid medium B. After this time, the particles were filtered to separate them from the liquid and were suspended in 24 mL of a 20 mM sodium citrate solution (liquid medium C).

### Example 68

40 mg of chlorphenamine maleate was suspended in 1.2 mL of canola oil with 1% v/v of liquid soy lecithin. This suspension was mixed with 4.8 mL of a 2% w/v alginate solution. The mixture of oily phase and aqueous phase was then emulsified by stirring. The resulting emulsion (liquid medium A) was added dropwise from a height of 6 cm over 24 mL of a 3% w/v CaCl₂ solution (liquid medium B), using a 10 mL syringe with an 18-gauge needle. The gel particles formed were stirred for approximately 10 minutes in liquid medium B. After this time, the particles were filtered to separate them from the liquid and were suspended in 24 mL of a 20 mM sodium citrate solution (liquid medium C).

### Example 69

40 mg of chlorphenamine maleate was suspended in 1.2 mL of water. This suspension was mixed with 4.8 mL of a 2% w/v alginate solution. Once the mixture (liquid medium A) was homogenized, it was added dropwise from a height of 6 cm over 24 mL of a solution comprises of 4.8 mL of a 1.0% w/v chitosan solution and 19.2 mL of a 7.5% w/v CaCl₂ solution (liquid medium B), using a 10 mL syringe with an 18-gauge needle. The gel particles formed were stirred for approximately 10 minutes in liquid medium B. After this time, the particles were filtered to separate them from the liquid and were suspended in 24 mL of 20 mM sodium citrate solution (liquid medium C).

### Example 70

40 mg of chlorphenamine maleate was suspended in 1.2 mL of canola oil with 1% v/v of liquid soy lecithin. This suspension was mixed with 4.8 mL of a 2% w/v alginate solution. The mixture of oily phase and aqueous phase was then emulsified by stirring. The resulting emulsion (liquid medium A) was added dropwise from a height of 6 cm over 24 mL of a 3% w/v CaCl₂ solution (liquid medium B), using a 10 mL syringe with an 18-gauge needle. The gel particles formed were stirred for approximately 10 minutes in liquid medium B. After this time, the particles were filtered to separate them from the liquid and were suspended in 24 mL of 20 mM sodium citrate solution (liquid medium C).

### Example 71

40 mg of chlorphenamine maleate were suspended in 1.2 mL of a 3% w/v CaCl₂ solution. This suspension was mixed with 4.8 mL of a 1.0% w/v chitosan solution. Once the mixture (liquid medium A) was homogenized, it was added dropwise from a height of 6 cm over 72 mL of a 0.83% w/v alginate solution (liquid medium B), using a 10 mL syringe with an 18-gauge needle. The gel particles formed were stirred for approximately 7 minutes in liquid medium B. After this time, the particles were filtered to separate them from the liquid and were suspended in 24 mL of 20 mM sodium citrate solution (liquid medium C).

### Example 72

40 mg of chlorphenamine maleate was suspended in 1.2 mL of water. This suspension was mixed with 4.8 mL of a 2% w/v gellan gum solution. Once the mixture (liquid medium A) was homogenized, it was added dropwise from a height of 6 cm over 24 mL of a CaCl₂ solution at 3% w/v (liquid medium B), using a 10 mL syringe with an 18-gauge needle. The gel particles formed were stirred for approximately 10 minutes in liquid medium B. After this time, the particles were filtered to separate them from the liquid and were suspended in 24 mL of 20 mM sodium citrate solution (liquid medium C).

### Example 73

40 mg of chlorphenamine maleate was suspended in 1.2 mL of canola oil with 1% v/v of liquid soy lecithin. This suspension was mixed with 4.8 mL of a 2% w/v gellan gum solution. The mixture of oily phase and aqueous phase was then emulsified by stirring. The resulting emulsion (liquid medium A) was added dropwise from a height of 6 cm over 24 mL of a 3% w/v CaCl₂ solution (liquid medium B), using a 10 mL syringe with an 18-gauge needle. The gel particles formed were stirred for approximately 10 minutes in liquid medium B. After this time, the particles were filtered to separate them from the liquid and were suspended in 24 mL of 20 mM sodium citrate solution (liquid medium C).

### Example 74

40 mg of chlorphenamine maleate was suspended in 1.2 mL of water. This suspension was mixed with 4.8 mL of a 2% w/v gellan gum solution. Once the mixture (liquid medium A) was homogenized, it was added dropwise from a height of 6 cm over 24 mL of a solution comprises of 4.8 mL of a 1.0% w/v chitosan solution and 19.2 mL of a 7.5% w/v CaCl₂ solution (liquid medium B), using a 10 mL syringe with an 18-gauge needle. The gel particles formed were stirred for approximately 10 minutes in liquid medium B. After this time, the particles were filtered to separate them from the liquid and were suspended in 24 mL of 20 mM sodium citrate solution (liquid medium C).

### Example 75

40 mg of chlorphenamine maleate was suspended in 1.2 mL of canola oil with 1% v/v of liquid soy lecithin. This suspension was mixed with 4.8 mL of a 2% w/v gellan gum solution. The mixture of oily phase and aqueous phase was then emulsified by stirring. The resulting emulsion (liquid medium A) was added dropwise from a height of 6 cm over 24 mL of a solution comprises of 4.8 mL of a 1.0% w/v chitosan solution and 19.2 mL of a 7.5% w/v CaCl₂ solution (liquid medium B), using a 10 mL syringe with an 18-gauge needle. The gel particles formed were stirred for approximately 10 minutes in liquid medium B. After this time, the particles were filtered to separate them from the liquid and were suspended in 24 mL of 20 mM sodium citrate solution (liquid medium C).

The results and observations of these examples are shown in Table 7.

TABLE 7
Characterization Particles Containing Chlorphenamine Maleate. Examples 67-75.

| | | | | | | | | Shape and | Color and |
|---|---|---|---|---|---|---|---|---|---|
| | | | | | chitosan | gauge | | size of gel | texture of |
| | gelling | water | Canola | CaCl₂ % | 1.0% | needle | Temp | particles | the gel |
| Example | agent | mL | oil, mL | w/v | w/v, mL | (G) | ° C. | (mm ± 0.4) | particles |
| 67 | Alginate | 1.2 | — | 3 | — | 18 | 25 | Spheroidal or | Transparent, |
| | 2.0% w/v | | | | | | | spheroidal | soft, stable |
| | | | | | | | | particles | |
| | | | | | | | | (+/−) | |
| | | | | | | | | 2.7 mm | |
| 68 | Alginate | — | 1.2 | 3 | — | 18 | 25 | Spheroidal or | Transparent, |
| | 2.0% w/v | | | | | | | spheroidal | soft, stable |
| | | | | | | | | particles | |
| | | | | | | | | (+/−) | |
| | | | | | | | | 2.6 mm | |
| 69 | Alginate | 1.2 | — | 3 | 4.8 | 18 | 25 | Spheroidal or | Transparent, |
| | 2.0% w/v | | | | | | | spheroidal | soft, stable |
| | | | | | | | | particles | |
| | | | | | | | | (+/−) | |
| | | | | | | | | 2.7 mm | |
| 70 | Alginate | — | 1.2 | 3 | 4.8 | 18 | 25 | Spheroidal or | Transparent, |
| | 2.0% w/v | | | | | | | spheroidal | soft, stable |
| | | | | | | | | particles | |
| | | | | | | | | (+/−) | |
| | | | | | | | | 2.7 mm | |
| 71 | Alginate | (72 mL) | — | 7.5 | 4.8 | 18 | 30 | Hollow | Transparent, |
| | 0.83% w/v | | | | | | | spherical or | soft, stable |
| | | | | | | | | spheroidal | |
| | | | | | | | | particles, ≅4.0 mm | |
| 72 | Gellan Gum | 1.2 | — | 3 | — | 18 | 40 | Spheres | White, |
| | 2% w/v | | | | | | | (+/−) | less soft |
| | | | | | | | | 2.89 mm | |
| 73 | Gellan Gum | — | 1.2 | 3 | — | 18 | 40 | Spheres | White, |
| | 2% w/v | | | | | | | (+/−) | Soft stable |
| | | | | | | | | 2.89 mm | |
| 74 | Gellan Gum | 1.2 | — | 3 | 4.8 | 18 | 40 | Spheres | White, |
| | 2% w/v | | | | | | | (+/−) | Soft stable |
| | | | | | | | | 2.89 mm | |
| 75 | Gellan Gum | — | 1.2 | 3 | 4.8 | 18 | 40 | Spheres | White, |
| | 2% w/v | | | | | | | (+/−) | Soft stable |
| | | | | | | | | 2.89 mm | |

The Weibull maximum concentration in liquid (FMax) and release rate of Higuchi models for chlorphenamine maleate, in liquid medium B and C for each of the above experiments is shown in Table 8.

TABLE 8
Chlorphenamine Maleate Release for Examples 67-70 and 72-75.

| | Maximum | Release rate | Maximum | Release rate |
|---|---|---|---|---|
| | concentration | in liquid | concentration | in liquid |
| | in liquid B, | medium B, | in liquid C, | medium C, |
| Example | mg/mL | mg/mL/min | mg/mL | mg/mL/min |
| 67 | 54.62 | 9.31 | 5.86 | 1.07 |
| 68 | 53.77 | 8.72 | 5.01 | 0.86 |
| 69 | 30.93 | 5.23 | 6.02 | 1.08 |
| 70 | 39.73 | 5.86 | 6.13 | 1.01 |
| 72 | 49.44 | 7.69 | 6.92 | 1.23 |
| 73 | 52.46 | 8.81 | 7.26 | 1.25 |
| 74 | 55.43 | 8.63 | 8.99 | 1.36 |
| 75 | 61.95 | 8.20 | 7.37 | 1.22 |

E. Examples with Chloramphenicol as API

In the next set of examples, chloramphenicol was used as API contained within the spheroid gel particles obtained according to the methods described herein.

### Example 76

150 mg of chloramphenicol was suspended in 1.2 mL of water. This suspension was mixed with 4.8 mL of a 2% w/v alginate solution. Once the mixture (liquid medium A) was homogenized, it was added dropwise from a height of 6 cm over 24 mL of a CaCl₂ solution at 3% w/v (liquid medium B), using a 10 mL syringe with an 18-gauge needle. The gel particles formed were stirred for approximately 10 minutes in liquid medium B. After this time, the particles were filtered to separate them from the liquid and were suspended in 24 mL of a 20 mM sodium citrate solution (liquid medium C).

### Example 77

150 mg of chloramphenicol was suspended in 1.2 mL canola oil with 1% v/v liquid soy lecithin. This suspension was mixed with 4.8 mL of a 2% w/v alginate solution. The mixture of oily phase and aqueous phase was then emulsified by stirring. The resulting emulsion (liquid medium A) was added dropwise from a height of 6 cm over 24 mL of a 3% w/v CaCl₂ solution (liquid medium B), using a 10 mL syringe with an 18-gauge needle. The gel particles formed were stirred for approximately 10 minutes in liquid medium B. After this time, the particles were filtered to separate them from the liquid and were suspended in 24 mL of a 20 mM sodium citrate solution (liquid medium C).

### Example 78

150 mg of chloramphenicol was suspended in 1.2 mL of water. This suspension was mixed with 4.8 mL of a 2% w/v alginate solution. Once the mixture (liquid medium A) was homogenized, it was added dropwise from a height of 6 cm over 24 mL of a solution composed of 4.8 mL of a 1.0% w/v chitosan solution and 19.2 mL of a 7.5% w/v CaCl₂ solution (liquid medium B), using a 10 mL syringe with an 18-gauge needle. The gel particles formed were stirred for approximately 10 minutes in liquid medium B. After this time, the particles were filtered to separate them from the liquid and were suspended in 24 mL of a 20 mM sodium citrate solution (liquid medium C).

### Example 79

Chloramphenicol 150 mg was suspended in 1.2 mL canola oil with 1% v/v liquid soy lecithin. This suspension was mixed with 4.8 mL of a 2% w/v alginate solution. Once the mixture (liquid medium A) was homogenized, it was added dropwise from a height of 6 cm over 24 mL of a solution composed of 4.8 mL of a 1.0% w/v chitosan solution and 19.2 mL of a 7.5% w/v CaCl₂ solution (liquid medium B), using a 10 mL syringe with an 18-gauge needle. The gel particles formed were stirred for approximately 10 minutes in liquid medium B. After this time, the particles were filtered to separate them from the liquid and were suspended in 24 mL of a 20 mM sodium citrate solution (liquid medium C).

### Example 80

150 mg of chloramphenicol was suspended in 1.2 mL of a 3% w/v CaCl₂ solution. This suspension was mixed with 4.8 mL of a 1.0% w/v chitosan solution. Once the mixture (liquid medium A) was homogenized, it was added dropwise from a height of 6 cm over 72 mL of a 0.83% w/v alginate solution (liquid medium B), using a 10 mL syringe with an 18-gauge needle. The gel particles formed were stirred for approximately 7 minutes in liquid medium B. After this time, the particles were filtered to separate them from the liquid and were suspended in 24 mL of 20 mM sodium citrate solution (liquid medium C).

The results and observations of these example were shown in Table 9.

TABLE 9
Characterization Particles Containing Chloramphenicol. Examples 76-80.

| | | | | | | | | Shape and | Color and |
|---|---|---|---|---|---|---|---|---|---|
| | | | | | chitosan | gauge | | size of gel | texture of |
| | gelling | water | Canola | CaCl₂ % | 1.0% | needle | Temp. | particles | the gel |
| Example | agent | mL | oil, mL | w/v | w/v, mL | (G) | ° C. | (mm ± 0.4) | particles |
| 76 | Alginate | 1.2 | — | 3 | — | 18 | 25 | Spheroidal or | Transparent, |
| | 2.0% w/v | | | | | | | spheroidal | soft, stable |
| | | | | | | | | particles | |
| | | | | | | | | (+/−) | |
| | | | | | | | | 2.8 mm | |
| 77 | Alginate | — | 1.2 | 3 | — | 18 | 25 | Spheroidal or | Transparent, |
| | 2.0% w/v | | | | | | | spheroidal | soft, stable |
| | | | | | | | | particles | |
| | | | | | | | | (+/−) | |
| | | | | | | | | 2.7 mm | |
| 78 | Alginate | 1.2 | — | 3 | 4.8 | 18 | 25 | Spheroidal or | Transparent, |
| | 2.0% w/v | | | | | | | spheroidal | soft, stable |
| | | | | | | | | particles | |
| | | | | | | | | (+/−) | |
| | | | | | | | | 2.6 mm | |
| 79 | Alginate | — | 1.2 | 3 | 4.8 | 18 | 25 | Spheroidal or | Transparent, |
| | 2.0% w/v | | | | | | | spheroidal | soft, stable |
| | | | | | | | | particles | |
| | | | | | | | | (+/−) | |
| | | | | | | | | 2.6 mm | |
| 80 | Alginate | (72 mL) | — | 7.5 | 4.8 | 18 | 30 | Hollow | Transparent, |
| | 0.83% w/v | | | | | | | spherical of | soft, stable |
| | | | | | | | | spheroidal | |
| | | | | | | | | particles, ≅4.0 mm | |

Examples 76 to 79 resulted in gel particles in the form of micromatrix, while example 5 resulted in gel particles in the form of microcapsules.

The Weibull maximum concentration in liquid (Fmax) and release rate of Higuchi models for chloramphenicol, in liquid medium B and C for each of the above experiments is shown in Table 10.

TABLE 10
Chloramphenicol Release for Examples 76-79.

| | Maximum | Release rate | Maximum | Release rate |
|---|---|---|---|---|
| | concentration | in liquid | concentration | in liquid |
| | in liquid B, | medium B, | in liquid C, | medium C, |
| Example | mg/mL | mg/mL/min | mg/mL | mg/mL/min |
| 76 | 105.62 | 8.73 | 57.80 | 3.13 |
| 77 | 73.55 | 9.24 | 64.02 | 2.76 |
| 78 | 71.21 | 8.65 | 59.70 | 3.62 |
| 79 | 61.11 | 7.23 | 255.57 | 2.61 |

F. Examples with Acetyl Salicylic Acid as API

In the next set of examples chlorphenamine maleate was used as an API contained within the spheroid gel particles obtained according to the methods described herein.

### Example 81

100 mg of acetyl salicylic acid was suspended in 1.2 mL of water. This suspension was mixed with 4.8 mL of a 2% w/v alginate solution. Once the mixture (liquid medium A) was homogenized, it was added dropwise from a height of 6 cm over 24 mL of a CaCl₂ solution at 3% w/v (liquid medium B) at room temperature, using a 10 mL syringe with an 18-gauge needle. The gel particles formed were stirred for approximately 10 minutes in liquid medium B. After this time, the particles were filtered to separate them from the liquid and were suspended in 24 mL of a 20 mM sodium citrate solution (liquid medium C).

### Example 82

100 mg of acetyl salicylic acid was suspended in 1.2 mL of canola oil with 1% v/v of liquid soy lecithin. This suspension was mixed with 4.8 mL of a 2% w/v alginate solution. The mixture of oily phase and aqueous phase was then emulsified by stirring. The resulting emulsion (liquid medium A) was added dropwise from a height of 6 cm over 24 mL of a 3% w/v CaCl₂ solution (liquid medium B) at room temperature, using a 10 mL syringe with an 18-gauge needle. The gel particles formed were stirred for approximately 10 minutes in liquid medium B. After this time, the particles were filtered to separate them from the liquid and were suspended in 24 mL of a 20 mM sodium citrate solution (liquid medium C).

### Example 83

100 mg of acetyl salicylic acid was suspended in 1.2 mL of water. This suspension was mixed with 4.8 mL of a 2% w/v alginate solution. Once the mixture (liquid medium A) was homogenized, it was added dropwise from a height of 6 cm over 24 mL of a solution composed of 4.8 mL of a 1.0% w/v chitosan solution and 19.2 mL of a 7.5% w/v CaCl₂ solution (liquid medium B) at room temperature, using a 10 mL syringe with an 18-gauge needle. The gel particles formed were stirred for approximately 10 minutes in liquid medium B. After this time, the particles were filtered to separate them from the liquid and were suspended in 24 mL of a 20 mM sodium citrate solution (liquid medium C).

### Example 84

100 mg of acetyl salicylic acid was suspended in 1.2 mL of canola oil with 1% v/v of liquid soy lecithin. This suspension was mixed with 4.8 mL of a 2% w/v alginate solution. Once the mixture (liquid medium A) was homogenized, it was added dropwise from a height of 6 cm over 24 mL of a solution composed of 4.8 mL of a 1.0% w/v chitosan solution and 19.2 mL of a 7.5% w/v CaCl₂ solution (liquid medium B) at room temperature, using a 10 mL syringe with an 18-gauge needle. The gel particles formed were stirred for approximately 10 minutes in liquid medium B. After this time, the particles were filtered to separate them from the liquid and were suspended in 24 mL of a 20 mM sodium citrate solution (liquid medium C).

### Example 85

100 mg of acetyl salicylic acid was suspended in 1.2 mL of water. This suspension was mixed with 4.8 mL of a 2% w/v alginate solution. Once the mixture (liquid medium A) was homogenized, it was added dropwise from a height of 6 cm over 24 mL of a CaCl₂ solution at 3% w/v (liquid medium B), using a 10 mL syringe with a 27-gauge needle. The gel particles formed were stirred for approximately 10 minutes in liquid medium B. After this time, the particles were filtered to separate them from the liquid and were suspended in 24 mL of a 20 mM sodium citrate solution (liquid medium C).

### Example 86

100 mg of acetyl salicylic acid was suspended in 1.2 mL of canola oil with 1% v/v of liquid soy lecithin. This suspension was mixed with 4.8 mL of a 2% w/v alginate solution. The mixture of oily phase and aqueous phase was then emulsified by stirring. The resulting emulsion (liquid medium A) was added dropwise from a height of 6 cm over 24 mL of a 3% w/v CaCl₂ solution (liquid medium B), using a 10 mL syringe with a 27-gauge needle. The gel particles formed were stirred for approximately 10 minutes in liquid medium B. After this time, the particles were filtered to separate them from the liquid and were suspended in 24 mL of a 20 mM sodium citrate solution (liquid medium C).

### Example 87

100 mg of acetyl salicylic acid was suspended in 1.2 mL of water. This suspension was mixed with 4.8 mL of a 2% w/v alginate solution. Once the mixture (liquid medium A) was homogenized, it was added dropwise from a height of 6 cm over 24 mL of a solution composed of 4.8 mL of a 1.0% w/v chitosan solution and 19.2 mL of a 7.5% w/v CaCl₂ solution (liquid medium B) at room temperature, using a 10 mL syringe with a 27-gauge needle. The gel particles formed were stirred for approximately 10 minutes in liquid medium B. After this time, the particles were filtered to separate them from the liquid and were suspended in 24 mL of a 20 mM sodium citrate solution (liquid medium C).

### Example 88

100 mg of acetyl salicylic acid was suspended in 1.2 mL of canola oil with 1% v/v of liquid soy lecithin. This suspension was mixed with 4.8 mL of a 2% w/v alginate solution. Once the mixture (liquid medium A) was homogenized, it was added dropwise from a height of 6 cm over 24 mL of a solution composed of 4.8 mL of a 1.0% w/v chitosan solution and 19.2 mL of a 7.5% w/v CaCl₂ solution (liquid medium B) at room temperature, using a 10 mL syringe with a 27-gauge needle. The gel particles formed were stirred for approximately 10 minutes in liquid medium B. After this time, the particles were filtered to separate them from the liquid and were suspended in 24 mL of a 20 mM sodium citrate solution (liquid medium C).

### Example 89

100 mg of acetyl salicylic acid was suspended in 1.2 mL of water. This suspension was mixed with 4.8 mL of a 2% w/v gellan gum solution. Once the mixture (liquid medium A) was homogenized, it was heated to 40° C. and was added dropwise from a height of 6-10 cm over 24 mL of a 3% w/v CaCl₂ solution (liquid medium B) at room temperature, using a 10 mL syringe with an 18-gauge needle, always maintaining the temperature of the liquid medium A at 40° C. throughout the dripping. The gel particles formed were stirred for approximately 10 minutes in liquid medium B. After this time, the particles were filtered to separate them from the liquid and were suspended in 24 mL of a 20 mM sodium citrate solution (liquid medium C).

### Example 90

100 mg of acetyl salicylic acid was suspended in 1.2 mL of canola oil with 1% v/v liquid soy lecithin. This suspension was mixed with 4.8 mL of a 2% w/v gellan gum solution. Next, the mixture of oily phase and aqueous phase was emulsified by stirring, heating it to 40° C. The resulting emulsion (liquid medium A) was added dropwise from a height of 6 cm over 24 mL of a 3% w/v CaCl₂ solution (liquid medium B) at room temperature, using a 10 mL syringe with an 18-gauge needle, always maintaining the temperature of the liquid medium A at 40° C. throughout the dripping. The gel particles formed were stirred for approximately 10 minutes in liquid medium B. After this time, the particles were filtered to separate them from the liquid and were suspended in 24 mL of a 20 mM sodium citrate solution (liquid medium C).

### Example 91

100 mg of acetyl salicylic acid was suspended in 1.2 mL of water. This suspension was mixed with 4.8 mL of a 2% w/v gellan gum solution. Once the mixture (liquid medium A) was homogenized, it was added dropwise from a height of 6 cm over 24 mL of a solution composed of 4.8 mL of a 1.0% w/v chitosan solution and 19.2 mL of a 7.5% w/v CaCl₂ solution (liquid medium B) at room temperature, using a 10 mL syringe with an 18-gauge needle. The gel particles formed were stirred for approximately 10 minutes in liquid medium B. After this time, the particles were filtered to separate them from the liquid and were suspended in 24 mL of a 20 mM sodium citrate solution (liquid medium C).

### Example 92

100 mg of acetyl salicylic acid was suspended in 1.2 mL of canola oil with 1% v/v liquid soy lecithin. This suspension was mixed with 4.8 mL of a 2% w/v gellan gum solution. Once the mixture (liquid medium A) was homogenized, it was added dropwise from a height of 6 cm over 24 mL of a solution composed of 4.8 mL of a 1.0% w/v chitosan solution and 19.2 mL of a 7.5% w/v CaCl₂ solution (liquid medium B), using a 10 mL syringe with an 18-gauge needle. The gel particles formed were stirred for approximately 10 minutes in liquid medium B. After this time, the particles were filtered to separate them from the liquid and were suspended in 24 mL of a 20 mM sodium citrate solution (liquid medium C).

The results and observations of these examples are shown in Table 11.

TABLE 11
Characterization Particles Containing Acetyl Salicylic Acid. Examples 81-92.

| | | | | | | | | Shape and | Color and |
|---|---|---|---|---|---|---|---|---|---|
| | | | | | chitosan | gauge | | size of gel | texture of |
| | gelling | water | Canola | CaCl₂ % | 1.0% | needle | Temp | particles | the gel |
| Example | agent | mL | oil, mL | w/v | w/v, mL | (G) | ° C. | (mm ± 0.4) | particles |
| 81 | Alginate | 1.2 | — | 3 | — | 18 | 25 | Spheroidal | Transparent, |
| | 2.0% w/v | | | | | | | (+/−)2.70 | soft, stable |
| | | | | | | | | mm | |
| 82 | Alginate | — | 1.2 | 3 | — | 18 | 25 | Spheroidal | Transparent, |
| | 2.0% w/v | | | | | | | (+/−) | soft, stable |
| | | | | | | | | 2.65 mm | |
| 83 | Alginate | 1.2 | — | 3 | 4.8 | 18 | 25 | Spheroidal | Transparent, |
| | 2.0% w/v | | | | | | | (+/−) | soft, stable |
| | | | | | | | | 2.70 mm | |
| 84 | Alginate | — | 1.2 | 3 | 4.8 | 18 | 25 | Spheroidal | Transparent, |
| | 2.0% w/v | | | | | | | (+/−) | soft, stable |
| | | | | | | | | 2.70 mm | |
| 85 | Alginate | 1.2 | — | 3 | — | 27 | 25 | Spheroidal | Transparent, |
| | 2.0% w/v | | | | | | | 0.3-0.5 mm | soft, stable |
| 86 | Alginate | — | 1.2 | 3 | — | 27 | 25 | Spheroidal | Transparent, |
| | 2.0% w/v | | | | | | | 0.3-0.5 mm | soft, stable |
| 87 | Alginate | 1.2 | — | 3 | 4.8 | 27 | 25 | Spheroidal | Transparent, |
| | 2.0% w/v | | | | | | | (+/−) | soft, stable |
| | | | | | | | | 2.70 mm | |
| 88 | Alginate | — | 1.2 | 3 | 4.8 | 27 | 25 | Spheroidal | Transparent, |
| | 2.0% w/v | | | | | | | (+/−) | soft, stable |
| | | | | | | | | 2.70 mm | |
| 89 | Gellan Gum | 1.2 | — | 3 | — | 18 | 40 | Spheres | White, |
| | 2% w/v | | | | | | | (+/−) | less soft |
| | | | | | | | | 2.89 mm | |
| 90 | Gellan Gum | — | 1.2 | 3 | — | 18 | 40 | Spheres | White, |
| | 2% w/v | | | | | | | (+/−) | Soft stable |
| | | | | | | | | 2.89 mm | |
| 91 | Gellan Gum | 1.2 | — | 3 | 4.8 | 18 | 40 | Spheres | White, |
| | 2% w/v | | | | | | | (+/−) | Soft stable |
| | | | | | | | | 2.89 mm | |
| 92 | Gellan Gum | — | 1.2 | 3 | 4.8 | 18 | 40 | Spheres | White, |
| | 2% w/v | | | | | | | (+/−) | Soft stable |
| | | | | | | | | 2.89 mm | |

The Weibull maximum concentration in liquid (Fmax) and release rate of Higuchi models for acetyl salicylic acid, in liquid medium B and C for each of the above experiments is shown in Table 12.

TABLE 12
Acetyl Salicylic Acid Release for Examples 81-92.

| | Maximum | Release rate | Maximum | Release rate |
|---|---|---|---|---|
| | concentration | in liquid | concentration | in liquid |
| | in liquid B, | medium B, | in liquid C, | medium C, |
| Example | mg/mL | mg/mL/min | mg/mL | mg/mL/min |
| 81 | 46.63 | 7.45 | 75.4 | 2.22 |
| 82 | 55.72 | 8.43 | 27.52 | 3.49 |
| 83 | 55.81 | 9.66 | 9.30 | 1.55 |
| 84 | 61.84 | 10.20 | 24.10 | 2.23 |
| 85 | 43.65* | N.D. | 29.15 | 4.82 |
| 86 | 39.20* | N.D. | 27.36 | 4.75 |
| 87 | 31.80* | N.D. | 18.30 | 2.29 |
| 88 | 41.90* | N.D. | 15.56 | 3.10 |
| 89 | 27.34 | 4.62 | 13.35 | 1.64 |
| 90 | 22.41 | 3.49 | 8.07 | 1.37 |
| 91 | 31.24 | 5.01 | 9.37 | 1.60 |
| 92 | 47.98 | 7.59 | 12.11 | 1.71 |

(N.D.): not determined;
*at 10 min only, other samples were measured at 60 or 70 minutes.

G. Examples with Ibuprofen as API

In the next set of examples ibuprofen was used as an API contained within the spheroid gel particles obtained according to the methods described herein.

### Example 93

100 mg of ibuprofen was suspended in 1.2 mL of water. This suspension was mixed with 4.8 mL of a 2% w/v alginate solution. Once the mixture (liquid medium A) was homogenized, it was added dropwise from a height of 6 cm over 24 mL of a CaCl₂ solution at 3% w/v (liquid medium B) at room temperature, using a 10 mL syringe with an 18-gauge needle. The gel particles formed were stirred for approximately 10 minutes in liquid medium B. After this time, the particles were filtered to separate them from the liquid and were suspended in 24 mL of a 20 mM sodium citrate solution (liquid medium C).

### Example 94

100 mg of ibuprofen was suspended in 1.2 mL of canola oil with 1% v/v of liquid soy lecithin. This suspension was mixed with 4.8 mL of a 2% w/v alginate solution. The mixture of oily phase and aqueous phase was then emulsified by stirring. The resulting emulsion (liquid medium A) was added dropwise from a height of 6 cm over 24 mL of a 3% w/v CaCl₂ solution (liquid medium B) at room temperature, using a 10 mL syringe with an 18-gauge needle. The gel particles formed were stirred for approximately 10 minutes in liquid medium B. After this time, the particles were filtered to separate them from the liquid and were suspended in 24 mL of a 20 mM sodium citrate solution (liquid medium C).

### Example 95

100 mg of ibuprofen was suspended in 1.2 mL of water. This suspension was mixed with 4.8 mL of a 2% w/v alginate solution. Once the mixture (liquid medium A) was homogenized, it was added dropwise from a height of 6 cm over 24 mL of a solution composed of 4.8 mL of a 1.0% w/v chitosan solution and 19.2 mL of a 7.5% w/v CaCl₂ solution (liquid medium B) at room temperature, using a 10 mL syringe with an 18-gauge needle. The gel particles formed were stirred for approximately 10 minutes in liquid medium B. After this time, the particles were filtered to separate them from the liquid and were suspended in 24 mL of a 20 mM sodium citrate solution (liquid medium C).

### Example 96

100 mg of ibuprofen was suspended in 1.2 mL of canola oil with 1% v/v of liquid soy lecithin. This suspension was mixed with 4.8 mL of a 2% w/v alginate solution. The mixture of oily phase and aqueous phase was then emulsified by stirring. The resulting emulsion (liquid medium A) was added dropwise from a height of 6 cm over 24 mL of a 3% w/v CaCl₂ solution (liquid medium B) at room temperature, using a 10 mL syringe with an 18-gauge needle. The gel particles formed were stirred for approximately 10 minutes in liquid medium B. After this time, the particles were filtered to separate them from the liquid and were suspended in 24 mL of a 20 mM sodium citrate solution (liquid medium C).

The results and observations of these examples are shown in Table 13.

TABLE 13
Characterization Particles Containing Ibuprofen. Examples 93-96.

| | | | | | | | | Shape and | Color and |
|---|---|---|---|---|---|---|---|---|---|
| | | | | | chitosan | gauge | | size of gel | texture of |
| | gelling | water | Canola | CaCl₂ % | 1.0% | needle | Temp | particles | the gel |
| Example | agent | mL | oil, mL | w/v | w/v, mL | (G) | ° C. | (mm ± 0.4) | particles |
| 93 | Alginate | 1.2 | — | 3 | — | 18 | 25 | Spheroidal | Transparent, |
| | 2.0% w/v | | | | | | | (+/−) | soft, stable |
| | | | | | | | | 2.60 mm | |
| 94 | Alginate | — | 1.2 | 3 | — | 18 | 25 | Spheroidal | Transparent, |
| | 2.0% w/v | | | | | | | (+/−) | soft, stable |
| | | | | | | | | 2.6 mm | |
| 95 | Alginate | 1.2 | — | 3 | 4.8 | 18 | 25 | Spheroidal | Transparent, |
| | 2.0% w/v | | | | | | | (+/−) | soft, stable |
| | | | | | | | | 2.60 mm | |
| 96 | Alginate | — | 1.2 | 3 | 4.8 | 18 | 25 | Spheroidal | Transparent, |
| | 2.0% w/v | | | | | | | (+/−) | soft, stable |
| | | | | | | | | 2.60 mm | |

The Weibull maximum concentration in liquid (Fmax) and release rate of Higuchi models for ibuprofen, in liquid medium B and C for each of the above experiments was shown in Table 14. To the samples 3 and 4 no API release were observed (release <1%).

TABLE 14
Ibuprofen Release for Examples 93 and 94.

| | Maximum | Release rate | Maximum | Release rate |
|---|---|---|---|---|
| | concentration | in liquid | concentration | in liquid |
| | in liquid B, | medium B, | in liquid C, | medium C, |
| Example | mg/mL | mg/mL/min | mg/mL | mg/mL/min |
| 93 | 18.77 | 1.97 | 19.29 | 2.17 |
| 94 | 20.84 | 3.67 | 21.63 | 2.77 |

H. Examples with Cannabidiol (CBD) as API

In the next set of examples 10% cannabinoid diol (CBD) in olive oil was used as API contained within the spheroid gel particles obtained according to the methods described herein.

### Example 97

40 mg of CBD (10% in olive oil) was suspended in 0.8 mL of canola oil with 1% v/v liquid soy lecithin. This suspension was mixed with 4.8 mL of a 2% w/v alginate solution. The mixture of oily phase and aqueous phase was then emulsified by stirring. The resulting emulsion (liquid medium A) was added dropwise from a height of 6 cm over 24 mL of a 3% w/v CaCl₂ solution (liquid medium B), using a 10 mL syringe with an 18-gauge needle. The gel particles formed were stirred for approximately 10 minutes in liquid medium B. After this time, the particles were filtered to separate them from the liquid and were suspended in 24 mL of a 20 mM CaCl₂ solution (liquid medium C).

### Example 98

80 mg of CBD (10% in olive oil) was suspended in 0.4 mL of canola oil with 1% v/v liquid soy lecithin. This suspension was mixed with 4.8 mL of a 2% w/v alginate solution. The mixture of oily phase and aqueous phase was then emulsified by stirring. The resulting emulsion (liquid medium A) was added dropwise from a height of 6 cm over 24 mL of a 3% w/v CaCl₂ solution (liquid medium B), using a 10 mL syringe with an 18-gauge needle. The gel particles formed were stirred for approximately 10 minutes in liquid medium B. After this time, the particles were filtered to separate them from the liquid and were suspended in 24 mL of a 20 mM CaCl₂ solution (liquid medium C).

### Example 99

150 mg of CBD (10% in olive oil) was mixed with 4.2 mL of a 2% w/v alginate solution. Once the mixture (liquid medium A) was homogenized, it was added dropwise from a height of 6 cm over 24 mL of a solution comprises of 4.8 mL of a 1.0% w/v chitosan solution and 19.2 mL of a 7.5% w/v CaCl₂ solution (liquid medium B), using a 10 mL syringe with an 18-gauge needle. The gel particles formed were stirred for approximately 10 minutes in liquid medium B. After this time, the particles were filtered to separate them from the liquid and were suspended in 24 mL of a 20 mM CaCl₂ solution (liquid medium C).

### Example 100

600 mg of CBD (10% in olive oil) was mixed with 3.9 mL of a 2% w/v alginate solution. Once the mixture (liquid medium A) was homogenized, it was added dropwise from a height of 6 cm to 24 mL of a solution comprises of 4.8 mL of a 1.0% w/v chitosan solution and 19.2 mL of a 7.5% w/v CaCl₂ solution (liquid medium B), using a 10 mL syringe with an 18-gauge needle. The gel particles formed were stirred for approximately 10 minutes in liquid medium B. After this time, the particles were filtered to separate them from the liquid and were suspended in 24 mL of a 20 mM CaCl₂ solution (liquid medium C).

The results and observations of these experiments are shown in Table 15.

TABLE 15
Characterization Particles Containing CBD. Examples 97-100.

| | | | | | | | | Shape and | Color and |
|---|---|---|---|---|---|---|---|---|---|
| | | | | | chitosan | gauge | | size of gel | texture of |
| | gelling | water | Canola | CaCl₂ % | 1.0% | needle | Temp. | particles | the gel |
| Example | agent | mL | oil, mL | w/v | w/v, mL | (G) | ° C. | (mm ± 0.4) | particles |
| 97 | Alginate | — | 1.2 | 3 | — | 18 | 25 | Spheroidal or | Transparent, |
| | 2.0% w/v | | | | | | | spheroidal | soft, stable |
| | | | | | | | | particles | |
| | | | | | | | | 2.75 mm | |
| 98 | Alginate | — | 0.8 | 3 | — | 18 | 25 | Spheroidal or | Transparent, |
| | 2.0% w/v | | | | | | | spheroidal | soft, stable |
| | | | | | | | | particles | |
| | | | | | | | | 2.74 mm | |
| 99 | Alginate | — | — | 3 | 4.8 | 18 | 25 | Spheroidal or | Transparent, |
| | 2.0% w/v | | | | | | | spheroidal | soft, stable |
| | | | | | | | | particles | |
| | | | | | | | | 2.65 mm | |
| 100 | Alginate | — | — | 3 | 4.8 | 18 | 25 | Spheroidal or | Transparent, |
| | 2.0% w/v | | | | | | | spheroidal | soft, stable |
| | | | | | | | | particles | |
| | | | | | | | | 2.69 mm | |

I. Examples with 9Δ-Tetrahydrocannabinol (THC) as API

In the next set of examples 30% 9Δ-tetrahydrocannabinol (THC) in hemp rosin was used as API contained within the spheroid gel particles obtained according to the methods described herein.

### Example 101

40 mg THC (30% in hemp rosin) was suspended in 1.2 mL canola oil with 1% v/v liquid soy lecithin. This suspension was mixed with 4.8 mL of a 2% w/v alginate solution. The mixture of oily phase and aqueous phase was then emulsified by stirring. The resulting emulsion (liquid medium A) was added dropwise from a height of 6 cm over 24 mL of a 3% w/v CaCl₂ solution (liquid medium B), using a 10 mL syringe with an 18-gauge needle. The gel particles formed were stirred for approximately 10 minutes in liquid medium B. After this time, the particles were filtered to separate them from the liquid and were suspended in 24 mL of a 20 mM CaCl₂ solution (liquid medium C).

### Example 102

80 mg THC (30% in hemp rosin) was suspended in 1.2 mL canola oil with 1% v/v liquid soy lecithin. This suspension was mixed with 4.8 mL of a 2% w/v alginate solution. The mixture of oily phase and aqueous phase was then emulsified by stirring. The resulting emulsion (liquid medium A) was added dropwise from a height of 6 cm over 24 mL of a 3% w/v CaCl₂ solution (liquid medium B), using a 10 mL syringe with an 18-gauge needle. The gel particles formed were stirred for approximately 10 minutes in liquid medium B. After this time, the particles were filtered to separate them from the liquid and were suspended in 24 mL of a 20 mM CaCl₂ solution (liquid medium C).

### Example 103

150 mg THC (30% in hemp rosin) was suspended in 1.2 mL canola oil with 1% v/v liquid soy lecithin. This suspension was mixed with 4.8 mL of a 2% w/v alginate solution. Once the mixture (liquid medium A) was homogenized, it was added dropwise from a height of 6 cm over 24 mL of a solution comprises of 4.8 mL of a 1.0% w/v chitosan solution and 19.2 mL of a 7.5% w/v CaCl2 solution (liquid medium B), using a 10 mL syringe with an 18-gauge needle. The gel particles formed were stirred for approximately 10 minutes in liquid medium B. After this time, the particles were filtered to separate them from the liquid and were suspended in 24 mL of a 20 mM CaCl2 solution (liquid medium C).

The results and observations of these example are shown in Table 16.

TABLE 16
Characterization Particles Containing THC. Examples 101-103.

| | | | | | | | | Shape and | Color and |
|---|---|---|---|---|---|---|---|---|---|
| | | | | | chitosan | gauge | | size of gel | texture of |
| | gelling | water | Canola | CaCl₂ % | 1.0% | needle | Temp. | particles | the gel |
| Example | agent | mL | oil, mL | w/v | w/v, mL | (G) | ° C. | (mm ± 0.4) | particles |
| 101 | Alginate | — | 1.2 | 3 | — | 18 | 25 | Spheroidal or | Transparent, |
| | 2.0% w/v | | | | | | | spheroidal | soft, stable |
| | | | | | | | | particles | |
| | | | | | | | | 2.75 mm | |
| 102 | Alginate | — | 1.2 | 3 | — | 18 | 25 | Spheroidal or | Transparent, |
| | 2.0% w/v | | | | | | | spheroidal | soft, stable |
| | | | | | | | | particles | |
| | | | | | | | | 2.59 mm | |
| 103 | Alginate | — | 1.2 | 3 | 4.8 | 18 | 25 | Spheroidal or | Transparent, |
| | 2.0% w/v | | | | | | | spheroidal | soft, stable |
| | | | | | | | | particles | |
| | | | | | | | | 2.69 mm | |

J. Examples with Combined Acetaminophen and Acetyl Salicylic Acid as APIs

In the next set of examples acetaminophen and acetyl salicylic acid as one combined form of APIs contained within the spheroid gel particles obtained according to the methods described herein was used.

### Example 104

100 mg of acetaminophen and 100 mg of acetyl salicylic acid were suspended in 2.4 mL of water. This suspension was mixed with 4.8 mL of a 2.5% w/v alginate solution. Once the mixture (liquid medium A) was homogenized, it was added dropwise from a height of 6-10 cm over 24 mL of a CaCl₂ solution at 3% w/v (liquid medium B) at room temperature, using a 10 mL syringe with an 18-gauge needle. The gel particles formed were stirred for approximately 10 minutes in liquid medium B. After this time, the particles were filtered to separate them from the liquid and were suspended in 24 mL of a 20 mM sodium citrate solution (liquid medium C).

### Example 105

100 mg of acetaminophen and 100 mg of acetyl salicylic acid were suspended in 2.4 mL of canola oil with 1% v/v liquid soy lecithin. This suspension was mixed with 4.8 mL of a 2.5% w/v alginate solution. Once the mixture (liquid medium A) was homogenized, it was added dropwise from a height of 6-10 cm over 24 mL of a CaCl₂ solution at 3% w/v (liquid medium B) at room temperature, using a 10 mL syringe with an 18-gauge needle. The gel particles formed were stirred for approximately 10 minutes in liquid medium B. After this time, the particles were filtered to separate them from the liquid and were suspended in 24 mL of a 20 mM sodium citrate solution (liquid medium C).

### Example 106

100 mg of acetaminophen and 100 mg of acetyl salicylic acid were suspended in 2.4 mL of 1.2 mL of water. This suspension was mixed with 4.8 mL of a 2.5% w/v alginate solution. The resulting emulsion (liquid medium A) was added dropwise from a height of 6 cm over 24 mL of a solution comprises of 4.8 mL of a 1.0% w/v chitosan solution and 19.2 mL of a 3% w/v CaCl₂ solution (liquid medium B) at room temperature, using a 10 mL syringe with an 18-gauge needle, always maintaining the temperature of liquid medium A at 40° C. throughout the drip. The gel particles formed were stirred for approximately 10 minutes in liquid medium B. After this time, the particles were filtered to separate them from the liquid and were suspended in 24 mL of a 20 mM sodium citrate solution (liquid medium C).

### Example 107

100 mg of acetaminophen and 100 mg of acetyl salicylic acid were suspended in 2.4 mL of canola oil with 1% v/v liquid soy lecithin. This suspension was mixed with 4.8 mL of a 2.5% w/v alginate solution. The mixture of oily phase and aqueous phase was then emulsified by stirring. The resulting emulsion (liquid medium A) was added dropwise from a height of 6 cm over 24 mL of a solution comprises of 4.8 mL of a 1.0% w/v chitosan solution and 19.2 mL of a 6% w/v CaCl₂ solution (liquid medium B) at room temperature, using a 10 mL syringe with an 18-gauge needle, always maintaining the temperature of liquid medium A at 40° C. throughout the drip. The gel particles formed were stirred for approximately 10 minutes in liquid medium B. After this time, the particles were filtered to separate them from the liquid and were suspended in 24 mL of a 20 mM sodium citrate solution (liquid medium C).

The results and observations of these examples are shown in Table 17.

TABLE 17
Characterization Particles Containing Acetaminophen and Acetyl Salicylic Acid. Examples 104-107.

| | | | | | | | | Shape and | Color and |
|---|---|---|---|---|---|---|---|---|---|
| | | | | | chitosan | gauge | | size of gel | texture of |
| | gelling | water | Canola | CaCl₂ % | 1.0% | needle | Temp | particles | the gel |
| Example | agent | mL | oil, mL | w/v | w/v, mL | (G) | ° C. | (mm ± 0.4) | particles |
| 104 | Alginate | 1.2 | — | 3 | — | 18 | 25 | Spheres | Transparent, |
| | 2.0% w/v | | | | | | | (+/−) | soft, stable |
| | | | | | | | | 2.89 mm | |
| 105 | Alginate | — | 1.2 | 3 | — | 18 | 25 | Spheres | Transparent, |
| | 2.0% w/v | | | | | | | (+/−) | soft, stable |
| | | | | | | | | 2.89 mm | |
| 106 | Alginate | 1.2 | — | 3 | 4.8 | 18 | 25 | Spheres | Transparent, |
| | 2.0% w/v | | | | | | | (+/−) | soft, stable |
| | | | | | | | | 2.89 mm | |
| 107 | Alginate | — | 1.2 | 3 | 4.8 | 18 | 25 | Spheres | Transparent, |
| | 2.0% w/v | | | | | | | (+/−) | soft, stable |
| | | | | | | | | 2.89 mm | |

The Weibull maximum concentration in liquid (Fmax) and release rate of Higuchi models for acetaminophen and acetyl salicylic acid, in liquid medium B and C for each of the above experiments is shown in Table 18.

TABLE 18
Acetyl Salicylic Acid (ASA) and Acetaminophen
(A) Release for Examples 104-107.

| | Maximum | | Release rate | | Maximum | | Release rate | |
|---|---|---|---|---|---|---|---|---|
| | concentration | | in liquid | | concentration | | in liquid | |
| | in liquid B, | | medium B, | | in liquid C, | | medium C, | |
| Example | mg/mL | | mg/mL/min | | mg/mL | | mg/mL/min | |
| APIs | A | ASA | A | ASA | A | ASA | A | ASA |
| 104 | 34.33 | 13.06 | N.D. | N.D. | 19.07 | 16.27 | 2.62 | 2.58 |
| 105 | 34.11 | 13.37 | N.D. | N.D. | 20.79 | 19.05 | 2.83 | 2.60 |
| 106 | 35.88 | 15.98 | N.D. | N.D. | 18.33 | 15.98 | 2.37 | 2.92 |
| 107 | 34.17 | 14.80 | N.D. | N.D. | 17.82 | 17.99 | 2.74 | 2.83 |

N.D.—not determined

The obtained results, have proved that these pharmaceutical formulations are viable to obtain APIs formulations that contain spheres suspended in a liquid.

### Dissolution Studies

Previous to the dissolution studies, it was necessary to obtain the appropriate formulation when the excipients were included in the final composition. In the present Examples, these assays were shown. Specifically, the assays related to obtaining the best ratio between the amount of APIs that remains within the matrix versus the components of the matrix (solution B). The excipients used were: methyl paraben as preservative, potassium sorbate as an antimicrobial and preservative drug and vitamin E (dl-alpha-tocopherol) as antioxidant. The preparation methods performed are described below.

Exemplary Pharmaceutical Formulation 1

140 mg of acetaminophen was suspended in 1.0 mL canola oil and 0.2 mL of vitamin E. This suspension was mixed with 4.8 mL of a 1.5% w/v alginate solution with 0.2% w/v methyl paraben and 0.1% w/v of potassium sorbate. The mixture of oily phase and aqueous phase was then emulsified by stirring. The resulting emulsion (liquid medium A) was added dropwise from a height of 6 cm over 24 mL of a 3% w/v CaCl₂ solution (liquid medium B), using a 10 mL syringe with an 18-gauge needle. The gel particles formed were stirred for approximately 10 minutes in liquid medium B. After the stirring step, the particles were filtered to separate them from the liquid and were suspended in 24 mL of a 20 mM sodium citrate solution (liquid medium C).

Exemplary Pharmaceutical Formulation 2

280 mg of acetaminophen was suspended in 1.0 mL canola oil and 0.2 mL of vitamin E. This suspension was mixed with 7.0 mL of a 1.5% w/v alginate solution with 0.2% w/v methyl paraben and 0.1% w/v of potassium sorbate. The mixture of oily phase and aqueous phase was then emulsified by stirring. The resulting emulsion (liquid medium A) was added dropwise from a height of 6 cm over 36 mL of a 3% w/v CaCl₂ solution (liquid medium B), using a 10 mL syringe with an 18-gauge needle. The gel particles formed were stirred for approximately 10 minutes in liquid medium B. After the stirring step, the particles were filtered to separate them from the liquid and were suspended in 24 mL of a 20 mM sodium citrate solution (liquid medium C).

Exemplary Pharmaceutical Formulation 3

560 mg of acetaminophen was suspended in 1.8 mL canola oil and 0.2 mL of vitamin E. This suspension was mixed with 8.0 mL of a 1.5% w/v alginate solution with 0.2% w/v methyl paraben and 0.1% w/v of potassium sorbate. The mixture of oily phase and aqueous phase was then emulsified by stirring. The resulting emulsion (liquid medium A) was added dropwise from a height of 6 cm over 54 mL of a 3% w/v CaCl₂ solution (liquid medium B), using a 10 mL syringe with an 18-gauge needle. The gel particles formed are stirred for approximately 10 minutes in liquid medium B. After the stirring step, the particles are filtered to separate them from the liquid and were suspended in 24 mL of a 20 mM sodium citrate solution (liquid medium C).

Finally, the real mass of acetaminophen for each pharmaceutical formulation (1, 2, or 3 above) was determined in the spheres at stage B. This was done by measuring the mg that remained in the CaCl₂ aqueous solution (B) at 5 and 10 min.

The results and observations for exemplary formulations 1, 2 and 3 are shown in Table 19.

TABLE 19
Quantification of Acetaminophen in Aqueous Media and Spheres
Media at 5 and 10 Minutes for Examples 1-3 (B Solution).

| | mg of | mg of | % of |
|---|---|---|---|
| | acetaminophen | acetaminophen | acetaminophen |
| Samples | in aqueous solution | in spheres | in spheres |
| (mg) | (B) at 5 min | (B) at 5 min | (B) at 5 min |
| 1 (140) | 52.42 | 87.58 | 62.56 |
| 2 (280) | 56.00 | 224.00 | 80.00 |
| 3 (560) | 57.33 | 502.67 | 89.76 |
| | mg of | mg of | % of |
| | acetaminophen | acetaminophen | acetaminophen |
| Samples | in aqueous solution | in spheres | in spheres |
| (mg) | (B) at 10 min | (B) at 10 min | (B) at 10 min |
| 1 (140) | 71.40 | 68.60 | 49.00 |
| 2 (280) | 56.66 | 203.34 | 72.62 |
| 3 (560) | 78.40 | 481.60 | 86.00 |

The Weibull maximum concentration in liquid (Fmax) and release rate of Higuchi models for acetaminophen in liquid medium C for each of the above experiments is shown in Table 20. While the percentage in the C medium and in the spheres are presented in Table 21.

TABLE 20
Obtained Maximum Concentration in Liquid (Fmax)
and Release Rate for Examples 1-3, (Solution C).

| Example | Maximum | |
|---|---|---|
| pharmaceutical | concentration in | Release rate in iquid |
| formulation | liquid C, mg/mL | medium C, mg/mL/min |
| 1 | 40.85 | 5.04 |
| 2 | 31.55 | 4.03 |
| 3 | 48.84 | 2.70 |

TABLE 21
Amount (%) of API in Aqueous Solution and Inside the
Spheres for Each of the Above Experiments at 70 Min.

| pharmaceutical formulation | % in aqueous C | % Spheres C |
|---|---|---|
| 1 | 33.4 | 66.6 |
| 2 | 27.5 | 72.5 |
| 3 | 20.2 | 79.8 |

### Additional Exemplary Formulation and Dissolution Studies

Dissolution studies are based on the quantification of API by means of spectrophotometry, for which a set of samples with different APIs was prepared. These were maintained in the absence and presence of light. Samples were taken over time, and quantified based on a calibration curve of a given API. The results are shown in Table 22 below.

TABLE 22
Ranges of amounts of API, in the spheres
and in the liquid medium after 2 h.

| | | | Estimated Time |
|---|---|---|---|
| | Liquid C | Spheres | to Equilibrium |
| | (Range)% | (%) | (min.) |
| Acetaminophen | | | |
| Alg/water | 15-30 | 70-85 | 50-60 |
| Alg/oil | 10-25 | 75-90 | 50-60 |
| alg/water/Ch | 15-25 | 75-85 | 50-60 |
| Alg/oil/Ch | 15-30 | 70-85 | 50-60 |
| Chlorphenamine | | | |
| maleate | | | |
| Alg/water | 4-10 | 90-96 | 30-40 |
| Alg/oil | 4-10 | 90-96 | 30-40 |
| Alg/water/Ch | 4-10 | 90-96 | 30-40 |
| Alg/oil/Ch | 4-10 | 90-96 | 30-40 |
| Chloramphenicol | | | |
| Alg/water | 40-50 | 50-60 | At 70 min diffusion |
| | | | continued |
| | | | (estimated that in |
| | | | 24 hours almost all |
| | | | the Chloramphenicol |
| | | | is the liquid) |
| Alg/oil | 40-50 | 50-60 | At 70 min diffusion |
| | | | continued |
| | | | (estimated that in |
| | | | 24 hours almost all |
| | | | the Chloramphenicol |
| | | | is the liquid) |
| Alg/water/Ch | 40-50 | 50-60 | At 70 min diffusion |
| | | | continued |
| | | | (estimated that in |
| | | | 24 hours almost all |
| | | | the Chloramphenicol |
| | | | is the liquid) |
| Alg/oil/Ch | 40-50 | 50-60 | At 70 min diffusion |
| | | | continued |
| | | | (estimated that in |
| | | | 24 hours almost all |
| | | | the Chloramphenicol |
| | | | is the liquid) |
| Acetylsalicylic | | | |
| Acid (ASA) | | | |
| Alg/water | 15-30 | 70-85 | 70-90 |
| Alg/oil | 15-30 | 70-85 | 70-90 |
| Alg/water/Ch | 10-25 | 75-90 | 70-90 |
| Alg/oil/Ch | 10-25 | 75-90 | 70-90 |
| Ibuprofen | | | |
| Alg/water | 15-25 | 75-85 | 70-90 |
| Alg/oil | 15-25 | 75-85 | 70-90 |
| Alg/water/Ch | 4-10 | 90-96 | 70-90 |
| Alg/oil/Ch | 4-10 | 90-96 | 70-90 |
| Naproxen | | | |
| Alg/water | 20-40 | 60-80 | 40-60 |
| Alg/oil | 20-40 | 60-80 | 40-60 |
| Alg/water/Ch | 20-40 | 60-80 | 40-60 |
| Alg/oil/Ch | 20-40 | 60-80 | 40-60 |

### Exemplary Pharmaceutical Formulations

Using the best conditions to obtain a pharmaceutical formulation, the following compositions were obtained including approximately 500 mg of acetaminophen as API.

### Example: Pharmaceutical Formulation A

1120 mg of acetaminophen was suspended in 3.6 mL canola oil and 0.4 mL of vitamin E. This suspension was mixed with 16 mL of a 1.5% w/v alginate solution with 0.2% w/v methyl paraben and 0.1% w/v of potassium sorbate. The mixture of oily phase and aqueous phase was then emulsified by stirring. The resulting emulsion (liquid medium A) was added dropwise from a height of 6 cm over 108 mL of a 3% w/v CaCl₂ solution (liquid medium B), using a peristaltic bomb at 18.8 rpm, with an 18-gauge needle. The gel particles formed are stirred for approximately 10 minutes in liquid medium B. After this time, the particles are filtered and washed with deionized water. Wet spheres were divided into two equal amounts and each of them were suspended in 50 mL of a 20 mM sodium citrate solution (liquid medium C).

### Example: Pharmaceutical Formulation B

1120 mg of acetaminophen was suspended in 3.6 mL canola oil and 0.4 mL of vitamin E. This suspension was mixed with 16 mL of a 1.5% w/v alginate solution with 0.2% w/v methyl paraben and 0.1% w/v of potassium sorbate. The mixture of oily phase and aqueous phase was then emulsified by stirring. Once the mixture (liquid medium A) was homogenized, it was added dropwise from a height of 6 cm over 108 mL of a solution comprises of 21.6 mL of a 1.0% w/v chitosan solution, 43.5 mL of a 7.5% w/v CaCl₂ solution and 42.75 mL of water (liquid medium B), using a peristaltic bomb at 18.8 rpm, with an 18-gauge needle. The gel particles formed were stirred for approximately 10 minutes in liquid medium B. After this time, the particles were filtered and washed with deionized water. Wet spheres were divided into two equal amounts and each of them were suspended in 50 mL of a 20 mM sodium citrate solution (liquid medium C).

The dissolution studies were carried out on ERWEKA DT light Series delivers, equipped with USP 2 paddle method, paddles at 50 rpm, the bath temperature was maintained at 37±0.5° C. The spheres and reference drugs were putting in 500 mL. Testing simulated intestinal fluid (pH 6.8), simulated duodenum fluid (pH 5.6) and stomach gastric fluid (pH 1.2), The time interval used for the drug release tests was 80 min.

To obtain the release profiles, the aqueous and solid components of formulation were separated in 50 mL of 20 mM sodium citrate solution and spheres. Then, the amount of acetaminophen was quantified for both cases.

TABLE 23
Example of Acetaminophen (A) Quantity Quantification
of the Solid and Aqueous Components of the
Proposed Pharmaceutical Formulation.

| Example | A (mg) in 50 mL | A (mg) in spheres | Total A (mg) in |
|---|---|---|---|
| pharmaceutical | of pharmaceutical | of pharmaceutical | pharmaceutical |
| formulation | formulation | formulation | formulation |
| pharmaceutical | 180.47 | 277.88 | 458.35 |
| formulation A_1 | | | |
| pharmaceutical | 183.36 | 276.34 | 459.70 |
| formulation A_2 | | | |
| pharmaceutical | 177.72 | 332.18 | 509.90 |
| formulation B_1 | | | |
| pharmaceutical | 148.84 | 349.30 | 498.14 |
| formulation B_2 | | | |

### Testing Simulated Intestinal Fluid (pH 6.8)

Release of API from the spheres in the pharmaceutical formulation was measured at pH 6.8. The buffer was prepared according to normative USP41-NF36_4492.

TABLE 24
Comparison of Acetaminophen of the Average Release
Parameters According to the Higuchi and Weibull
Models for Simulated Intestinal Fluid (pH 6.8).

| | Maximum | Release rate |
|---|---|---|
| | concentration | in liquid |
| | in liquid, mg/mL | medium, mg/mL/s |
| Example | (Weibull model) | (Higuchi, K_{H}) |
| pharmaceutical | 424.01 ± 1.51 | 52.55 ± 0.60 |
| formulation A | | |
| pharmaceutical | 407.72 ± 3.54 | 45.90 ± 0.80 |
| formulation B | | |
| Panadol ® | 489.30 | 80.68 |
| Paracetamol ® | 484.73 | 76.94 |
| Daytime Cold ® and | 314.53 | 43.81 |
| Flu Relief ® | | |
| TAPSIN ® chewable | 386.30 | 44.47 |

### Testing Simulated Duodenum Fluid (pH 5.6)

Release of API from the spheres in the pharmaceutical formulation was measured at pH 5.6. The buffer was prepared according to normative USP41-NF36_4492.

TABLE 25
Comparison of Acetaminophen of the Average Release
Parameters According to the Higuchi and Weibull
Models for Simulated Intestinal Fluid (pH 5.6).

| | Maximum | Release rate |
|---|---|---|
| | concentration | in liquid |
| | in liquid, mg/mL, | medium, mg/mL/s, |
| Example | Weibull model | Higuchi, K_{H} |
| pharmaceutical | 381.10 ± 1.5 | 63.26 ± 0.80 |
| formulation A | | |
| pharmaceutical | 375.27 ± 2.3 | 47.6 ± 0.50 |
| formulation B | | |
| Panadol ® | 2142 | 60.27 |
| Paracetamol ® Lab Chile | 495.21 | 78.14 |
| Daytime Cold ® and Flu | 335.25 | 47.53 |
| Relief ® | | |
| TAPSIN ® chewable | 638.66 | 41.94 |

### Testing Simulated Stomach Gastric Fluid (pH 1.2)

Release of API from the spheres in the pharmaceutical formulation was measured at pH 1.2. The buffer was prepared according to normative USP41-NF36_4492.

TABLE 26
Comparison of Acetaminophen of the Average Release
Parameters According to the Higuchi and Weibull
Models for Simulated Intestinal Fluid (pH 1.2).

| | Maximum | Release rate |
|---|---|---|
| | concentration | in liquid |
| | in liquid, mg/mL | medium, mg/mL/s |
| Example | (Weibull model) | (Higuchi, K_{H}) |
| pharmaceutical | 300.44 ± 5.3 | 42.88 ± 0.3 |
| formulation A | | |
| pharmaceutical | 410.07 ± 3.5 | 53.01 ± 1.3 |
| formulation B | | |
| Panadol ® | 485.84 | 81.60 |
| Paracetamol ® | 483.67 | 79.06 |
| Daytime Cold ® and Flu | 320.77 | 46.41 |
| Relief ® | | |
| TAPSIN ® chewable | 377.12 | 42.47 |

### Stability Studies

Stability testing was performed to provide evidence on how the quality of APIs change in the time function, under the effect of a different factors such as temperature, humidity and light, and to establish a shelf life for the drug product and the recommended storage conditions.

In particular, another important point to consider is the stability of the spheres suspended in the liquid medium C. It was determined that if the liquid medium C was 20 mM CaCl2, these spheres were stable for only 2-2.5 months, whereas when the spheres were suspended in medium liquid C (e.g., sodium citrate 20 mM), these spheres were stable for more than 4 months. By adding excipients such as vitamin E, methyl paraben and potassium sorbate, among others, the stability of the spheres was extended to more than 4-5 months. Finally, in the absence of light, the spheres were stable for longer than 7 months. It is important to note that the stability was also dependent on the chemical stability of the API itself.

TABLE 27
Stability Testing for Some APIs and Formulations Described Herein.

| | | C Liquid | | Rh | | | | Appearance |
|---|---|---|---|---|---|---|---|---|
| No | Sample | Medium | Temperature | (%) | Time | | Color | Spheres |
| 1 | Acetaminophen/Alg/ | 20 Mm Cacl₂ | 21° C. ± 2° C. | 30 | 60 | Days | Brown | Non-Uniform |
| | W | | | | | | | |
| 2 | Acetaminophen/Alg/ | 20 Mm Cacl₂ | 21° C. ± 2° C. | 30 | 60 | Days | Brown | Non-Uniform |
| | Oil | | | | | | | |
| 3 | Acetaminophen/Alg/ | 20 Mm Cacl₂ | 21° C. ± 2° C. | 30 | 60 | Days | Brown | Non-Uniform |
| | W/Ch | | | | | | | |
| 4 | Acetaminophen/Alg/ | 20 Mm Cacl₂ | 21° C. ± 2° C. | 30 | 60 | Days | Brown | Non-Uniform |
| | Oil/Ch | | | | | | | |
| 5 | Acetaminophen/Alg/ | 20 Mm Sodium | 21° C. ± 2° C. | 30 | 90 | Days | Brown | Uniform |
| | W | Citrate | | | | | | |
| 6 | Acetaminophen/Alg/ | 20 Mm Sodium | 21° C. ± 2° C. | 30 | 90 | Days | Brown | Uniform |
| | Oil | Citrate | | | | | | |
| 7 | Acetaminophen/Alg/ | 20 Mm Sodium | 21° C. ± 2° C. | 30 | 90 | Days | Brown | Uniform |
| | W/Ch | Citrate | | | | | | |
| 8 | Acetaminophen/Alg/ | 20 Mm Sodium | 21° C. ± 2° C. | 30 | 90 | Days | Brown | Uniform |
| | Oil/Ch | Citrate | | | | | | |
| 9 | Acetaminophen/Alg/ | 20 Mm Sodium | 25° C. ± 2° C. | 21 | 90 | Days | Brown | Uniform |
| | Water | Citrate | | | | | | |
| 10 | Acetaminophen/Alg/ | 20 Mm Sodium | 25° C. ± 2° C. | 21 | 90 | Days | Brown | Uniform |
| | Oil | Citrate | | | | | | |
| 11 | Acetaminophen/Alg/ | 20 Mm Sodium | 25° C. ± 2° C. | 21 | 90 | Days | Brown | Uniform |
| | W/Ch | Citrate | | | | | | |
| 12 | Acetaminophen/Alg/ | 20 mm Sodium | 25° C. ± 2° C. | 21 | 90 | Days | Brown | Uniform |
| | Oil/Ch | Citrate | | | | | | |
| 13 | Acetaminophen/Alg/ | 20 Mm Sodium | 5° C. ± 2° C. | 75 | 120 | Days | White | Uniform |
| | W | Citrate | | | | | | |
| 14 | Acetaminophen/Alg/ | 20 Mm Sodium | 5° C. ± 2° C. | 75 | 120 | Days | White | Uniform |
| | Oil | Citrate | | | | | | |
| 15 | Acetaminophen/Alg/ | 20 Mm Sodium | 5° C. ± 2° C. | 75 | 120 | Days | White | Uniform |
| | W/Ch | Citrate | | | | | | |
| 16 | Acetaminophen/Alg/ | 20 Mm Sodium | 5° C. ± 2° C. | 75 | 120 | Days | White | Uniform |
| | Oil/Ch | Citrate | | | | | | |
| 17 | Acetaminophen/Gg/ | 20 Mm Sodium | 25° C. ± 2° C. | 21 | 90 | Days | Light- | Uniform |
| | W | Citrate | | | | | Brown | |
| 18 | Acetaminophen/Gg/ | 20 Mm Sodium | 25° C. ± 2° C. | 21 | 90 | Days | Light- | Uniform |
| | Oil | Citrate | | | | | Brown | |
| 19 | Acetaminophen/Gg/ | 20 Mm Sodium | 25° C. ± 2° C. | 21 | 90 | Days | Light- | Uniform |
| | W/Ch | Citrate | | | | | Brown | |
| 20 | Acetaminophen/Gg/ | 20 Mm Sodium | 25° C. ± 2° C. | 21 | 90 | Days | Light- | Uniform |
| | Oil/Ch | Citrate | | | | | Brown | |
| 21 | MA/Alg/W | 20 Mm Sodium | 25° C. ± 2° C. | 21 | 90 | Days | White | Uniform |
| | | Citrate | | | | | | |
| 22 | MA/Alg/Oil | 20 Mm Sodium | 25° C. ± 2° C. | 21 | 90 | Days | White | Uniform |
| | | Citrate | | | | | | |
| 23 | MA/Alg/W/Ch | 20 Mm Sodium | 25° C. ± 2° C. | 21 | 90 | Days | White | Uniform |
| | | Citrate | | | | | | |
| 24 | MA/Alg/Oil/Ch | 20 Mm Sodium | 25° C. ± 2° C. | 21 | 90 | Days | White | Uniform |
| | | Citrate | | | | | | |
| 25 | MA/Alg/W | 20 Mm Sodium | 5° C. ± 2° C. | 75 | 120 | Days | White | Uniform |
| | | Citrate | | | | | | |
| 26 | MA/Alg/Oil | 20 Mm Sodium | 5° C. ± 2° C. | 75 | 120 | Days | White | Uniform |
| | | Citrate | | | | | | |
| 27 | MA/Alg/W/Ch | 20 Mm Sodium | 5° C. ± 2° C. | 75 | 120 | Days | White | Uniform |
| | | Citrate | | | | | | |
| 28 | MA/Alg/Oil/Ch | 20 Mm Sodium | 5° C. ± 2° C. | 75 | 120 | Days | White | Uniform |
| | | Citrate | | | | | | |
| 29 | ASA/Alg/W | 20 Mm Sodium | 25° C. ± 2° C. | 21 | 90 | Days | White | Uniform |
| | | Citrate | | | | | | |
| 30 | ASA/Alg/Oil | 20 Mm Sodium | 25° C. ± 2° C. | 21 | 90 | Days | White | Uniform |
| | | Citrate | | | | | | |
| 31 | ASA/Alg/W/Ch | 20 Mm Sodium | 25° C. ± 2° C. | 21 | 90 | Days | White | Uniform |
| | | Citrate | | | | | | |
| 32 | ASA/Alg/Oil/Ch | 20 Mm Sodium | 25° C. ± 2° C. | 21 | 90 | Days | White | Uniform |
| | | Citrate | | | | | | |
| 33 | ASA/Alg/W | 20 Mm Sodium | 5° C. ± 2° C. | 75 | 120 | Days | White | Uniform |
| | | Citrate | | | | | | |
| 34 | ASA/Alg/Oil | 20 Mm Sodium | 5° C. ± 2° C. | 75 | 120 | Days | White | Uniform |
| | | Citrate | | | | | | |
| 35 | ASA/Alg/W/Ch | 20 Mm Sodium | 5° C. ± 2° C. | 75 | 120 | Days | White | Uniform |
| | | Citrate | | | | | | |
| 36 | ASA/Alg/Oil/Ch | 20 Mm Sodium | 5° C. ± 2° C. | 75 | 120 | Days | White | Uniform |
| | | Citrate | | | | | | |
| 37 | CM/Alg/W | 20 Mm Sodium | 25° C. ± 2° C. | 21 | 90 | Days | White | Uniform |
| | | Citrate | | | | | | |
| 38 | CM/Alg/Oil | 20 Mm Sodium | 25° C. ± 2° C. | 21 | 90 | Days | White | Uniform |
| | | Citrate | | | | | | |
| 39 | CM/Alg/W/Ch | 20 Mm Sodium | 25° C. ± 2° C. | 21 | 90 | Days | White | Uniform |
| | | Citrate | | | | | | |
| 40 | CM/Alg/Oil/Ch | 20 Mm Sodium | 25° C. ± 2° C. | 21 | 90 | Days | White | Uniform |
| | | Citrate | | | | | | |
| 41 | CM/Alg/W | 20 Mm Sodium | 5° C. ± 2° C. | 75 | 120 | Days | White | Uniform |
| | | Citrate | | | | | | |
| 42 | CM/Alg/Oil | 20 Mm Sodium | 5° C. ± 2° C. | 75 | 120 | Days | White | Uniform |
| | | Citrate | | | | | | |
| 43 | CM/Alg/W/Ch | 20 Mm Sodium | 5° C. ± 2° C. | 75 | 120 | Days | White | Uniform |
| | | Citrate | | | | | | |
| 44 | CM/Alg/Oil/Ch | 20 Mm Sodium | 5° C. ± 2° C. | 75 | 120 | Days | White | Uniform |
| | | Citrate | | | | | | |
| 45 | C/Alg/W | 20 Mm Sodium | 25° C. ± 2° C. | 21 | 90 | Days | White | Uniform |
| | | Citrate | | | | | | |
| 46 | C/Alg/Oil | 20 Mm Sodium | 25° C. ± 2° C. | 21 | 90 | Days | White | Uniform |
| | | Citrate | | | | | | |
| 47 | C/Alg/W/Ch | 20 Mm Sodium | 25° C. ± 2° C. | 21 | 90 | Days | White | Uniform |
| | | Citrate | | | | | | |
| 48 | C/Alg/Oil/Ch | 20 Mm Sodium | 25° C. ± 2° C. | 21 | 90 | Days | White | Uniform |
| | | Citrate | | | | | | |
| 49 | C/Alg/W | 20 Mm Sodium | 5° C. ± 2° C. | 75 | 120 | Days | White | Uniform |
| | | Citrate | | | | | | |
| 50 | C/Alg/Oil | 20 Mm Sodium | 5° C. ± 2° C. | 75 | 120 | Days | White | Uniform |
| | | Citrate | | | | | | |
| 51 | C/Alg/W/Ch | 20 Mm Sodium | 5° C. ± 2° C. | 75 | 120 | Days | White | Uniform |
| | | Citrate | | | | | | |
| 52 | C/Alg/Oil/Ch | 20 Mm Sodium | 5° C. ± 2° C. | 75 | 120 | Days | White | Uniform |
| | | Citrate | | | | | | |
| 53 | Pharmaceutical | 20 Mm Sodium | 25° C. ± 2° C. | 21 | 120 | Days | White | Uniform |
| | Formulation A | Citrate | | | | | | |
| 54 | Pharmaceutical | 20 Mm Sodium | 5° C. ± 2° C. | 75 | 180 | Days | White | Uniform |
| | Formulation A | Citrate | | | | | | |
| 55 | Pharmaceutical | 20 Mm Sodium | 25° C. ± 2° C. | 21 | 120 | Days | White | Uniform |
| | Formulation B | Citrate | | | | | | |
| 56 | Pharmaceutical | 20 Mm Sodium | 5° C. ± 2° C. | 75 | 180 | Days | White | Uniform |
| | Formulation B | Citrate | | | | | | |

Alg: Sodium alginate;
GG; gellan gum;
W: Water
Ch: Chitosan;
MA: Mefenamic acid;
ASA: Acetyl salicylic acid;
C: Chloramphenicol

Effect of Incorporation of Emulsion and Different Gelling Agents in the Release of API from the Pharmaceutical Formulation

The release of the API from the spherical or spheroidal particles, as described herein can be controlled to modify the amount of API that is partitioned between the spherical- and/or spheroidal-shaped particles and the liquid medium containing them. Thus, the maximum amount of API that is transferred from the particles to the liquid medium B or, particularly, to the liquid medium C, can be controlled directly through the formulation of spherical- and/or spheroidal-shaped particles described herein. Additionally, the formulation of particles described herein allows altering the release rate of the API from the particles towards the liquid medium that contains them. Surprisingly, the composition of the liquid medium is also involved in obtaining the release rate as well as the maximum amount of API released.

This combination of factors allows to design and produce a particular system of spherical- and/or spheroidal-shaped particles following the methods described herein, in order to obtain a desired release rate and maximum for the release of API towards the liquid containing the particles. The release also depends directly on the physicochemical properties of the API, so the methods described herein can be used and adapted to formulate systems of particles contained in a liquid medium, specifically for each API of interest. Thus, each API may be formulated in different ways according to the methods described herein to obtain the desired effect and to obtain API stability.

The factors that can be modified to specifically formulate a delivery system for each API are described below. As a first factor, the use of different gelling agents according to the methods described herein makes it possible to control the release rate and the maximum release of the API in the liquid medium where the spherical- and/or spheroidal-shaped particles are contained. As a second factor, the incorporation of the API as part of an emulsion also makes it possible to control, in a surprising and not obvious way, the rate of release of the API and the maximum amount of API that can be released from the particles contained in a liquid. As a third factor, the composition of the liquid medium can also be manipulated in a way to cause an additional effect on these same factors. Finally, as a fourth factor, also the size of the particles can be modified to alter the release of the API.

An example of the surprising effects of modification of these formulation design factors is shown below for the same API (acetaminophen or paracetamol), as an example of the different effects that can be achieved using the same amount of API in all formulations (120 mg of total acetaminophen in each example). To demonstrate the effect of the liquid medium, the release of API from the same stabilized spherical- and/or spheroidal-shaped particles in liquid medium B and then transferred to liquid medium C was measured. It was shown that this change of medium causes an additional release, and different release rates from the particles, depending on their characteristics. Surprisingly, the release changes do not follow a specific pattern that would be predictable or obvious to someone skilled in the art.

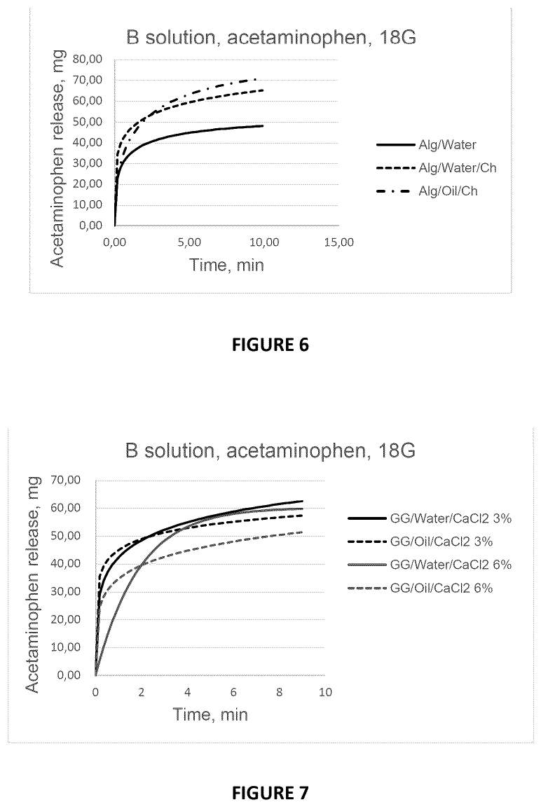

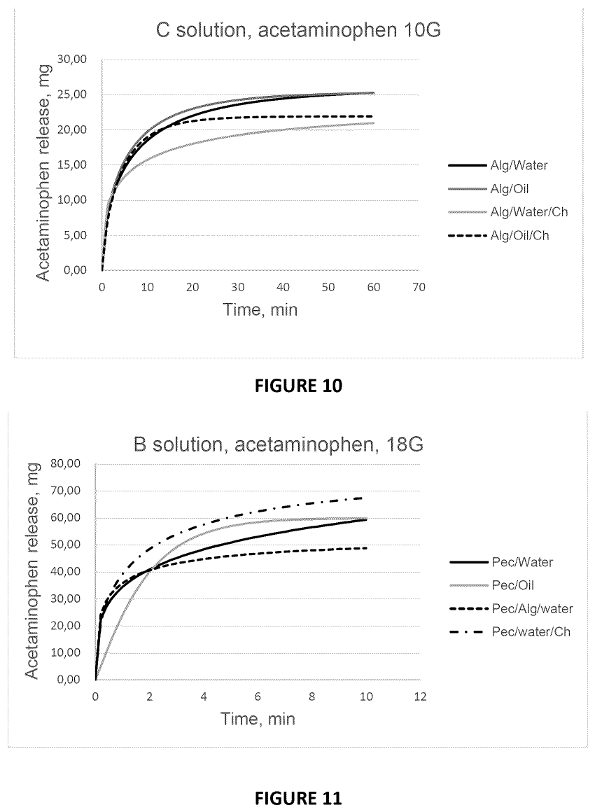

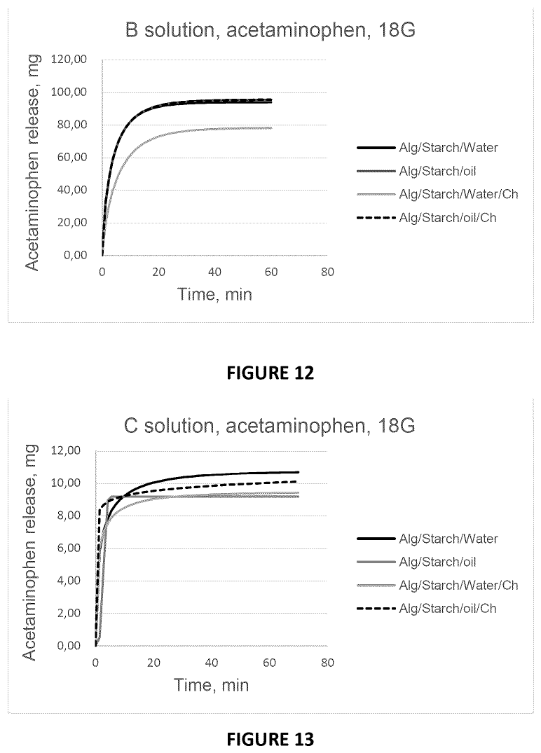

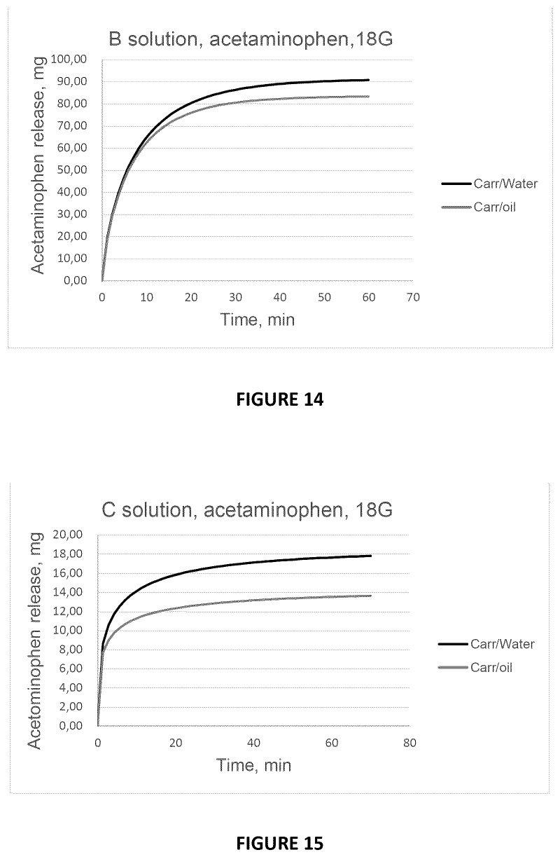

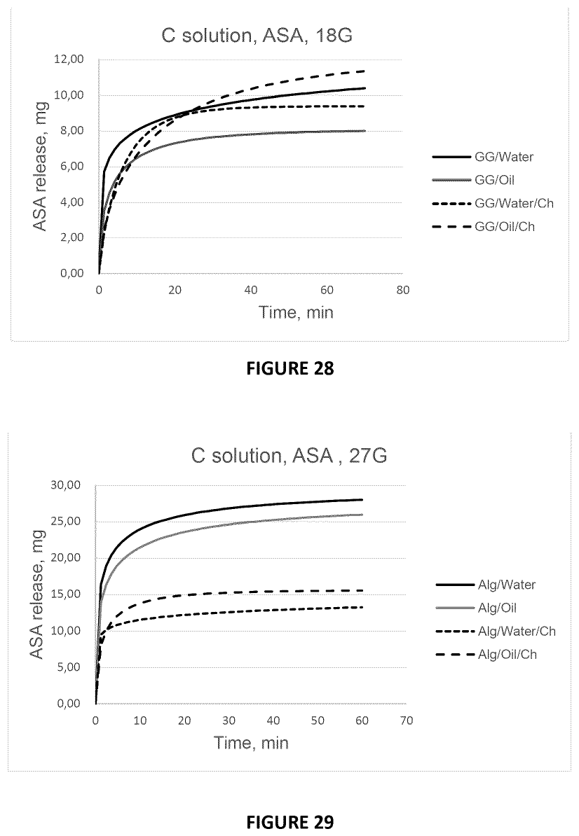

FIGS. 5 to 34 show the difference in shape of the acetaminophen release curves in two different liquid media. The particles were first manufactured following the methods described herein using a liquid medium B containing 3% w/v CaCl₂) (=0.21 M=210 mM). After coming to equilibrium the particles in medium B for approximately 40 minutes, the spherical- and/or spheroidal-shaped particles were transferred to liquid C containing 0.285% w/v CaCl2 (0.02 M=20 mM) or sodium citrate 20 mM. In this liquid C an additional portion of API is released, with different speed and a different release maximum. These characteristics depends on each particular combination of gelling agents and the size of particles.

In FIG. 5 to FIG. 34 it can be seen that the release maximum and release rate was obtained by combining the various factors that allow surprising behaviors to be obtained that are neither obvious nor predictable by one skilled in the art, that it depend on the nature of each API.

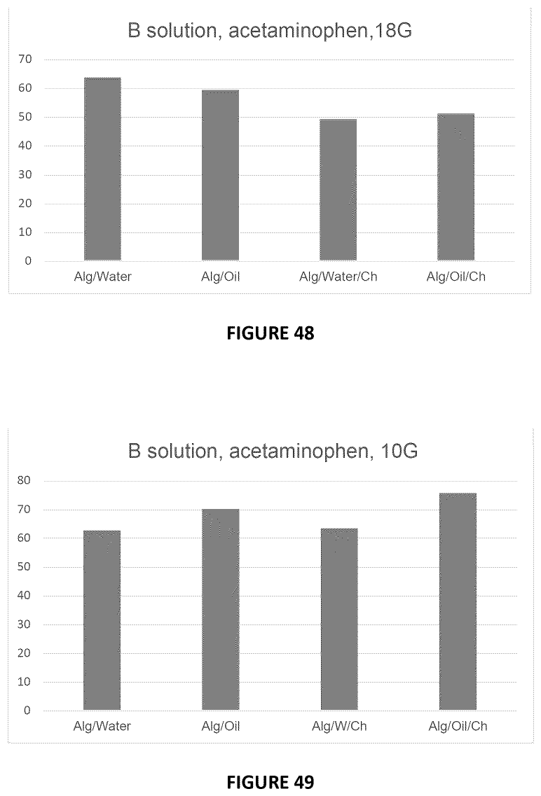

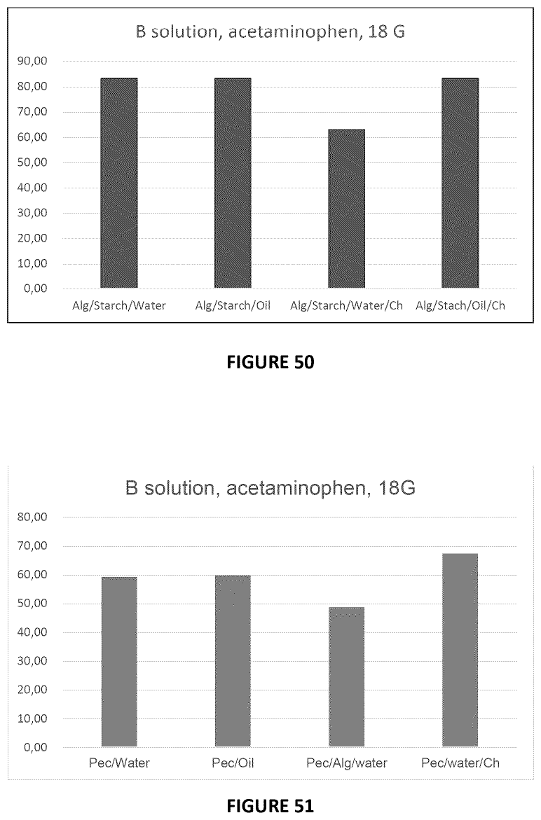

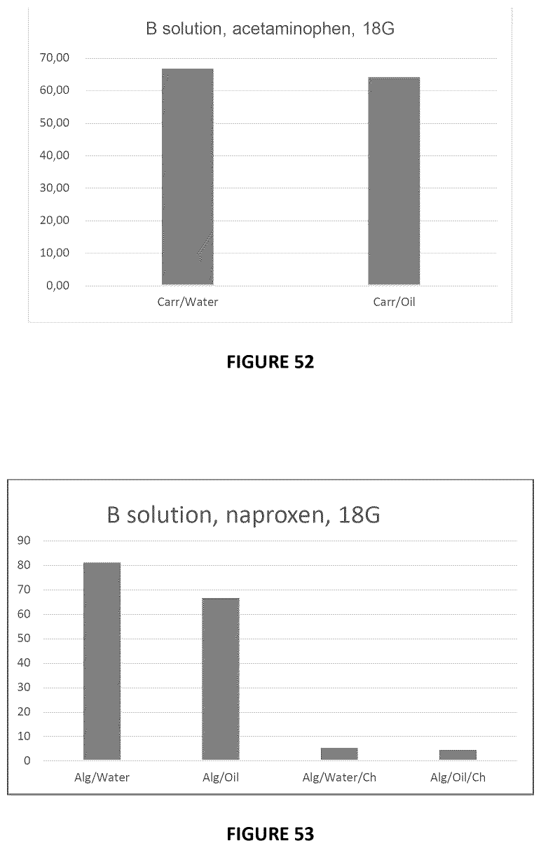

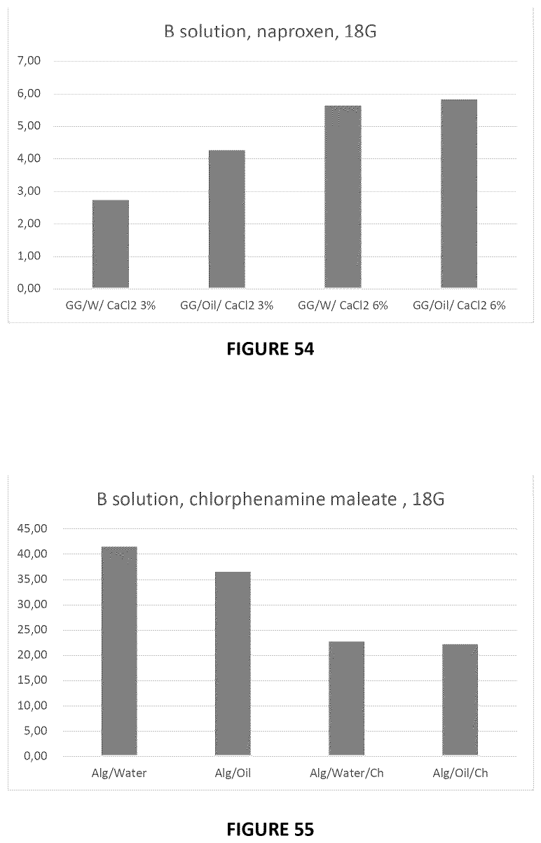

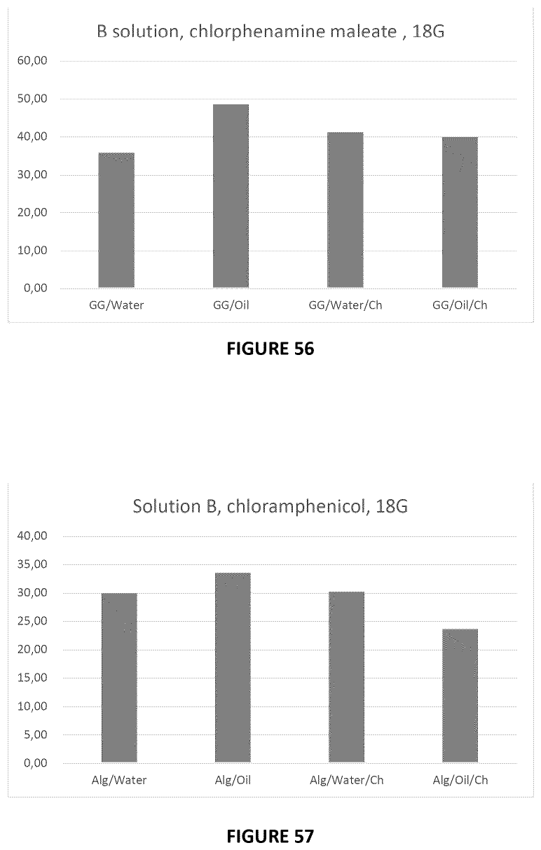

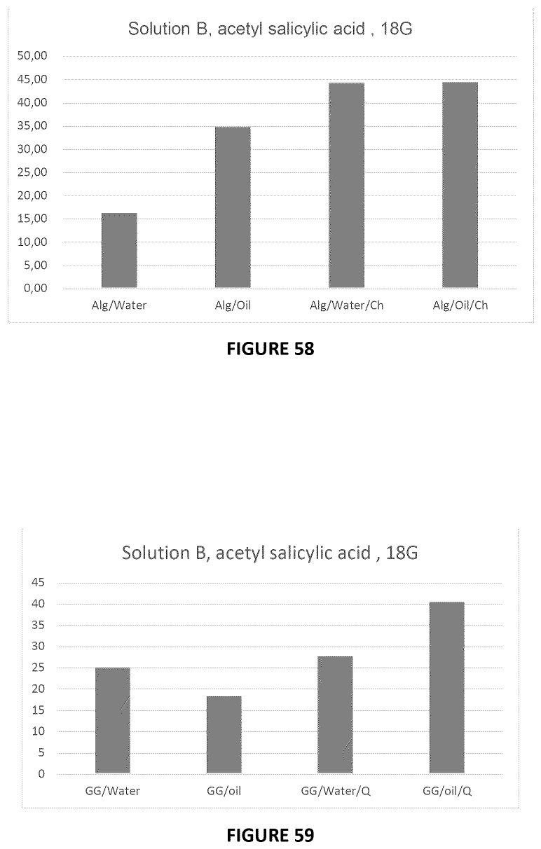

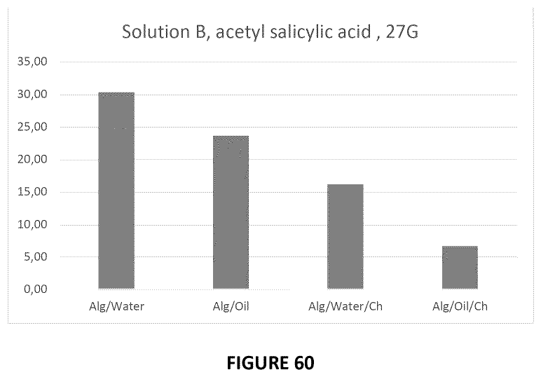

FIG. 47 to FIG. 60 compared the results for the release maximum obtained in each of the experiments, solution B, at 10 min of preparation of spheres.

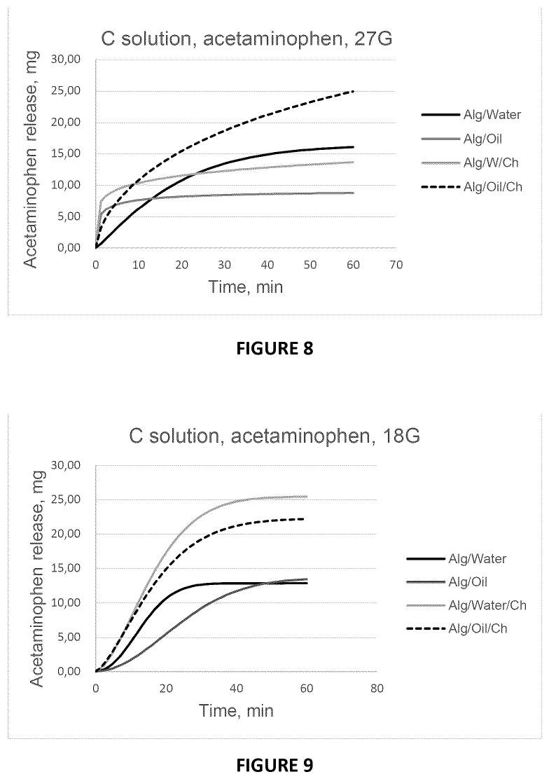

For combinations of gelling agents and acetaminophen as API, FIG. 5 to FIG. 7 show a similar release maximum in medium B, while in FIG. 8 to FIG. 10 release maximum with a variation of two times (minimum≈14 mg; maximum≈28 mg) can be observed in liquid medium C, by varying the type of gelling agent and the formulation of the API in the form of an emulsion in the spheric or spherical particles described herein. Additionally, it was shown that the particle size also influences the maximum release of API to the liquid medium containing the particles. Surprisingly, the effect of particle size was also not obvious, since spheroid alginate particles with emulsion formulated API that were larger in size (3-4 mm ø) release API to a greater extent than smaller spheroid particles (1-4 mm ø). This behavior was contrary to what one skilled in the art might have expected from traditional release models for active ingredients. The presence of emulsion also had an unexpected effect, since it increased the amount of API released to the liquid medium in which the spheroid particles of the example were contained, both in the case of liquid medium B and liquid medium C.

Furthermore, others combinations of gelling agents such as gellan gum, pectin, alginate/starch and carrageenan (see FIGS. 7, 11, 12, 13 and 14) were tested. The results showed that for pectin alginate/starches and carrageenan, these were not satisfactory since more than 80% of the API was released in the liquid medium B. While that for gellan gum, acetaminophen release was similar at alginate.

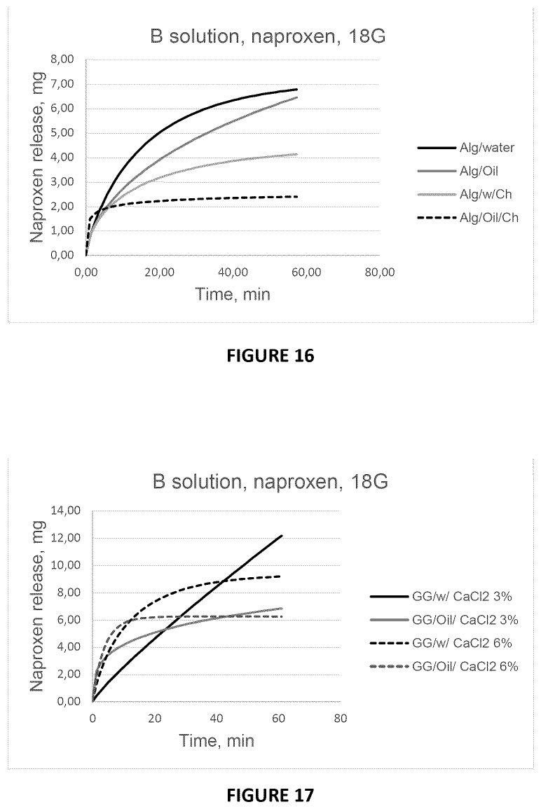

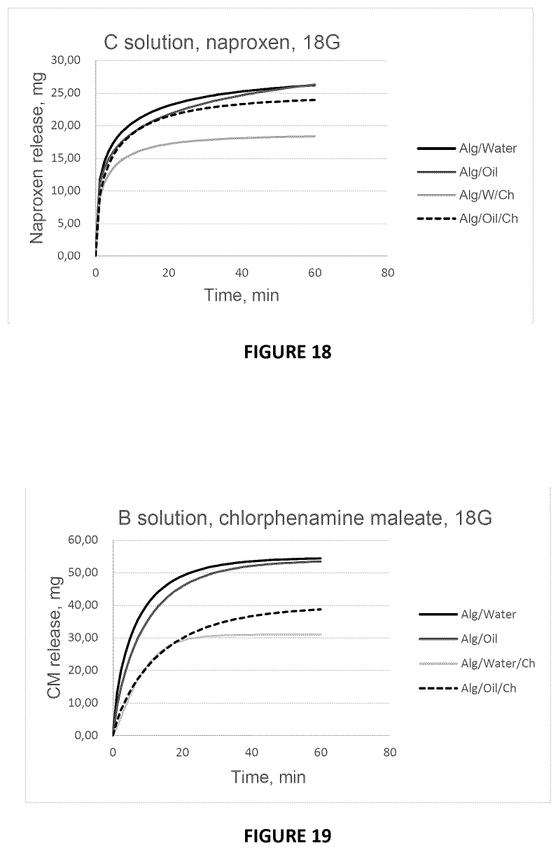

For combinations of gelling agents and naproxen, FIG. 16 to FIG. 18 show that, for similar release maximum in medium B, but in that case, it was possible to observe the formation of a calcium complex with naproxen. For this reason, two conditions of calcium chloride concentration were used to determine in which condition the formation of this precipitate is prevented (see Table 3). Once the sphere was formed (FIG. 18) release maximum with a variation of two times (minimum=17 mg; maximum=28 mg) can be observed in liquid medium C, by varying the type of gelling agent and the formulation of the API in the form of an emulsion in the spheric or spherical particles described herein. The presence of chitosan also had an unexpected and surprising effect, since it increased the amount of API released to the liquid medium in which the spheroid particles of the example were contained, both liquid medium B and liquid medium C.

Surprisingly, with mefenamic acid, no release was observed in any media (B or C). This API remains totally insoluble.

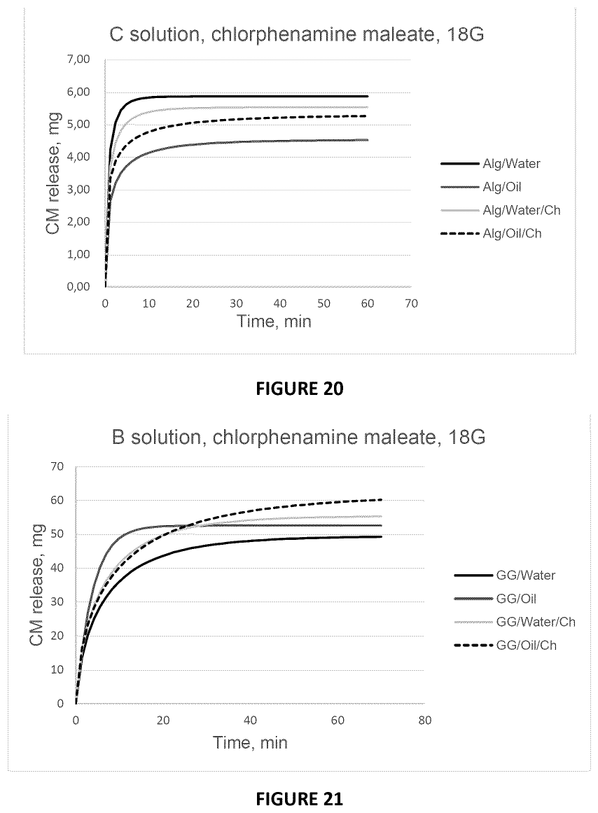

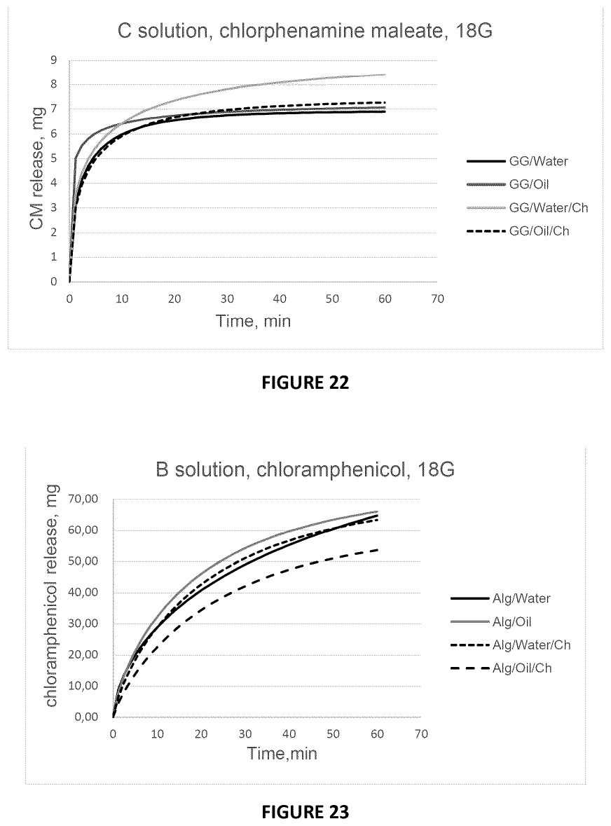

For combinations of gelling agents and chlorphenamine maleate, FIG. 19, FIG. 21 and FIG. 22 show similar release maximum in medium B, for alginate and gellan gum. On the other hand, FIGS. 20 and 22 demonstrate that release maximum with a variation of two times (minimum=4 mg; maximum=6 mg) can be observed in liquid medium C, by varying the type of gelling agent and the formulation of the API in the form of an emulsion in the spheric or spherical particles described herein. Additionally, it was shown that the particle size also influences the maximum release of API to the liquid medium containing the particles.

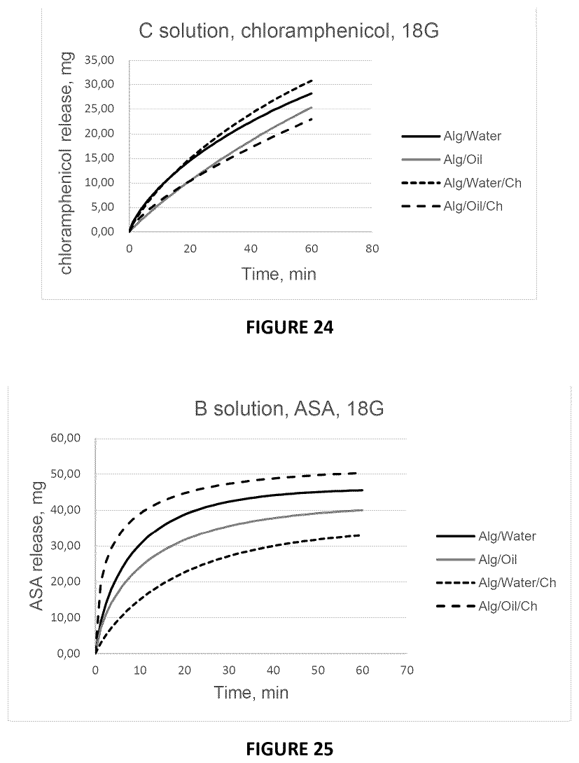

For combinations of gelling agents and chloramphenicol, e.g., FIG. 23, surprisingly showed a maintained release over time, for all combinations of alginate and chitosan, in B medium liquid. Once the spheres were suspended in the liquid medium C, chloramphenicol continued to be released (FIG. 24), and the presence of emulsion did not influence the release after one hour of being suspended—it continued to be released into the medium C liquid. Additionally, the presence of emulsion and/or chitosan also did not show an unexpected effect, since it increased the amount of API released into the liquid medium in which the particles of the example were contained, both in the case of liquid medium B and liquid medium C.

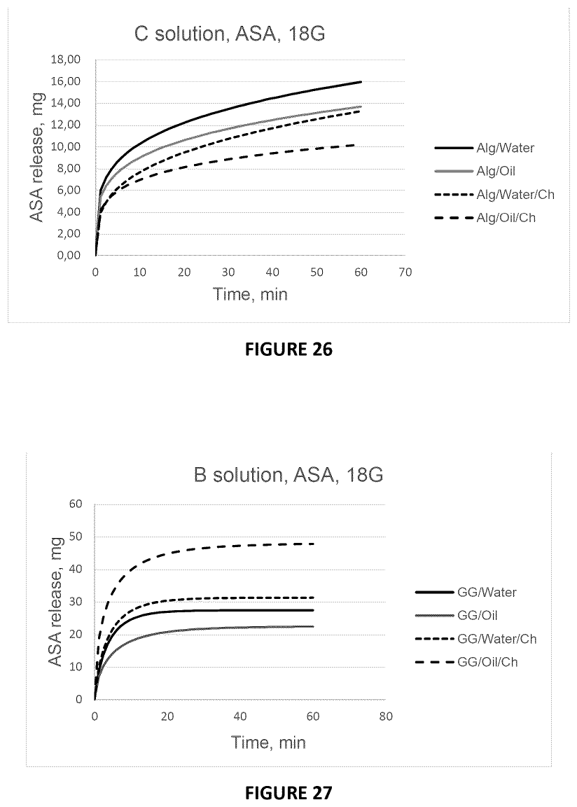

For combinations of gelling agents and acetyl salicylic acid, FIG. 25 and FIG. 27 show similar release maximum in medium B, for both alginate and gellan gum. While in FIGS. 26, 28 and 29 release maximum with a variation of two times (minimum=10 mg; maximum=30 mg) can be observed in liquid medium C, by varying the type of gelling agent, the gauge of needle and the formulation of the API in the spheric or spherical particles described herein.

In particular, in the liquid medium B, the particle size influenced the maximum release of API toward liquid medium containing the particles. Additionally, the presence of emulsion did not have an unexpected effect for both liquid medium B and liquid medium C. On the other hand, the incorporation of gelling agent chitosan had an unexpected effect, due to its increased release.

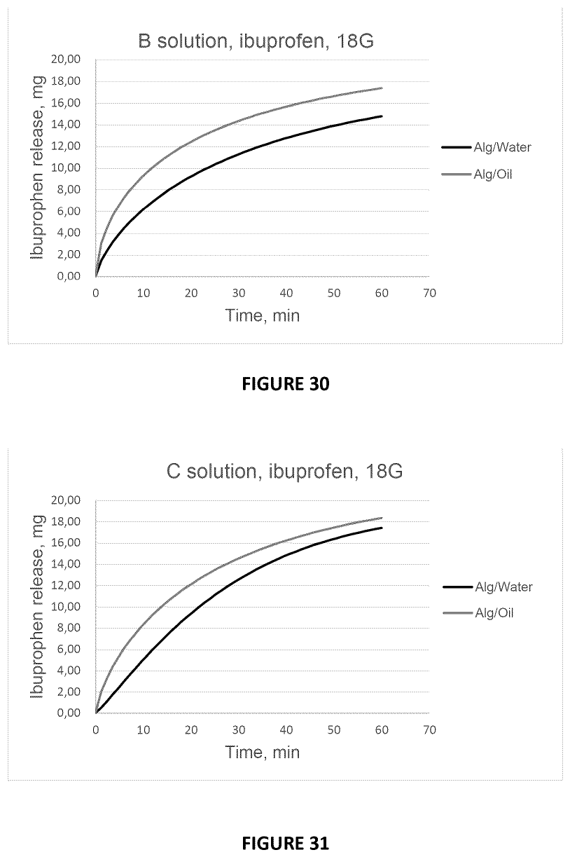

For sodium alginate and ibuprofen, FIG. 30 shows similar release maximum in medium B, for alginate/water and alginate/oil. While for the FIG. 31 release maximum with a variation of two times (minimum=16 mg; maximum=18 mg) can be observed in liquid medium C. In this case, the presence of emulsion did not influence the release after one hour of being suspended—it continued to be released into the medium C liquid.

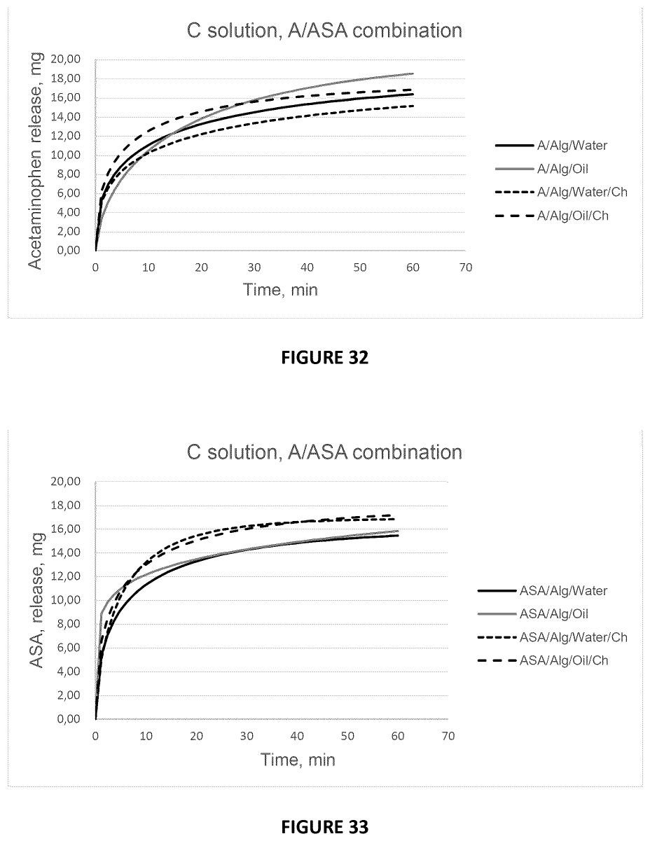

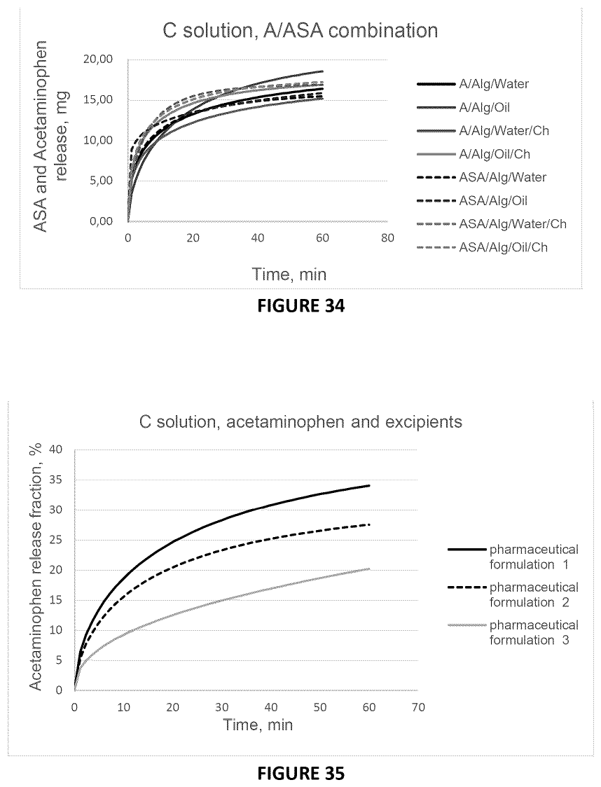

For combinations of gelling agents and, acetaminophen and acetyl salicylic acid, FIG. 32 and FIG. 33 show similar release maximum in medium C until 1 h. But it is important to note, acetaminophen had a continues released into the medium C liquid after 60 min, while for salicylic acid, after 40 min the release reached a constant point. When comparing the behavior of both APIs in the same matrix, FIG. 34, an independent behavior was observed. That means, the presence of one APIs did not modify the profile release of the other API.

It is interesting to note that in the aforementioned FIGS. 5 to 34, the maximum release and the release rate can be moderated according to the modification of factors such as: the gelling agent, the spherifying agent, the size of the spheres and/or spheroids. Thus, depending on the nature of the API, a desired pharmaceutical formulation can be proposed.

Release test was performed, to compare the effects of the described drug formulations with commercially available drugs, in different pH media. Firstly, a simulated pharmaceutical formulation with different amounts of acetaminophen (140 mg, 280 and 560 mg) was obtained that further contained methyl paraben, vitamin E and potassium sorbate excipients. Table 18 shows one of the surprising results of the described formulations because it was possible to modulate the amount of acetaminophen inside the sphere, by modifying the amount of gelling agent, when it was being formed in the liquid medium B. Surprisingly, at least 86% of API within the sphere was retained at 10 min of stirring (see release profile in FIG. 35).

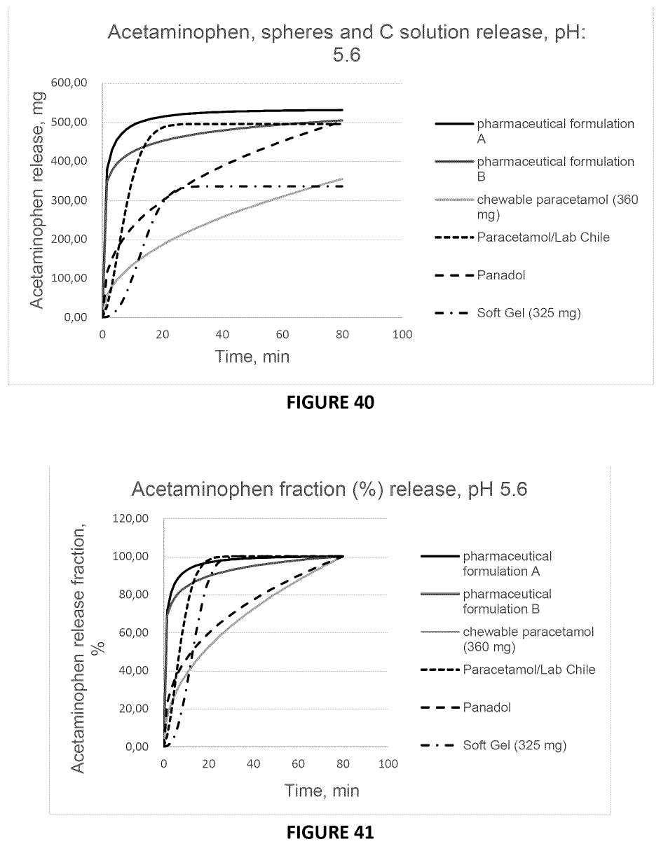

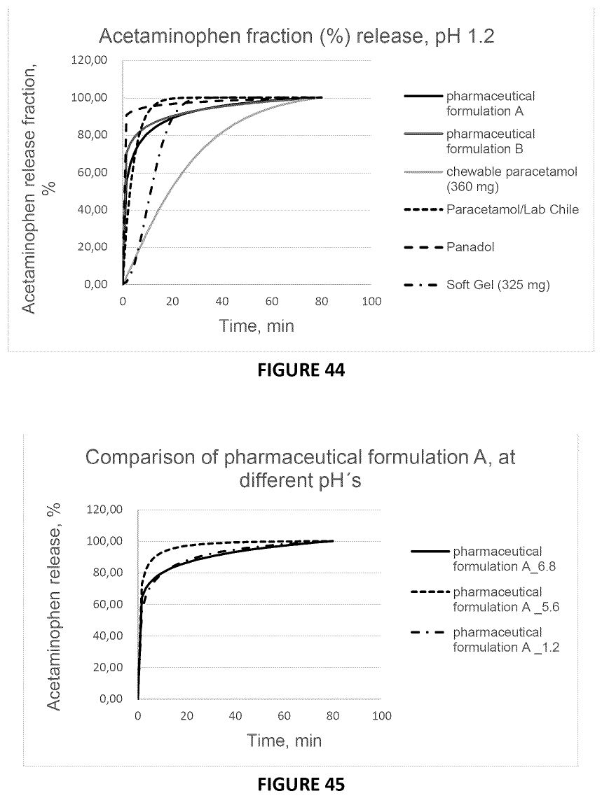

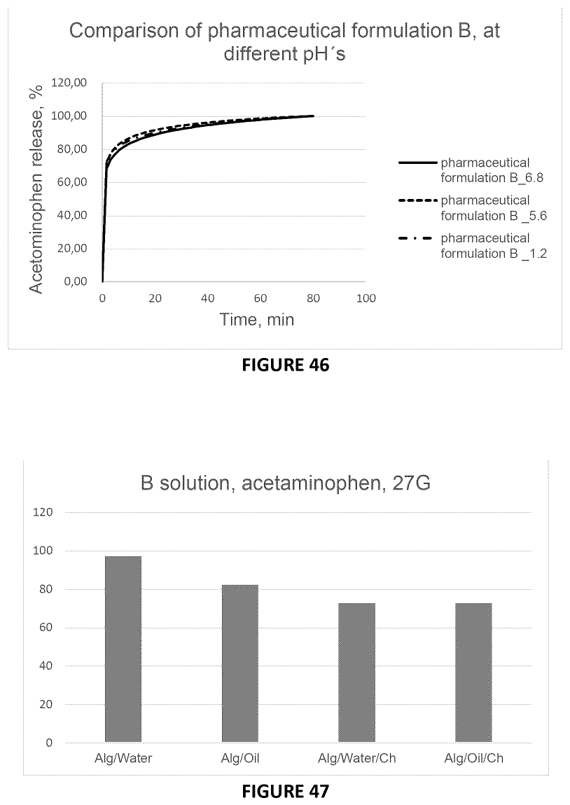

Considering the results disclosed in the previous paragraph, two exemplary formulations—A and B—can be proposed. Formulations A and B contain sodium alginate in oil and sodium alginate/chitosan in oil, and suitable excipients. Release test were performed and the tests showed that the formulations had a similar release profile as compared to the commercially available products (FIG. 35 to FIG. 44). Specifically, these commercially available products include micro gelatinized particles (Paracetamol/Lab Chile) and soft capsules (Soft Gel).

The spheres suspended in a liquid media, as described herein, firstly, the acetaminophen release (mg) only inside the spheres was measured. Second, the acetaminophen release in medium liquid C was measured at three different pH's. For example, in FIG. 43, to the stomach gastric fluid (pH 1.2), the total acetaminophen quantity (mg) in the formulation was obtained.

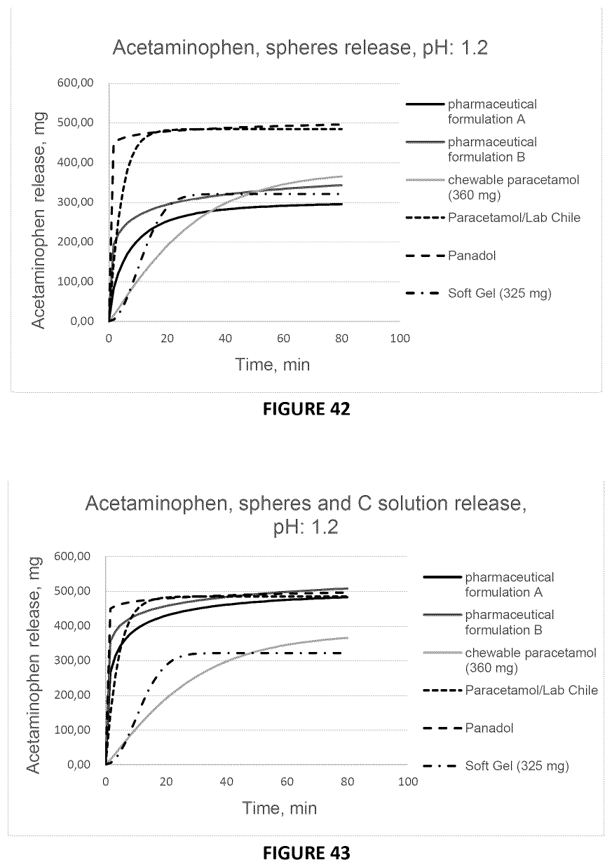

FIG. 42 shows at pH 1.2 (stomach gastric fluid), that practically 90% of the acetaminophen was released within 20 to 30 min. Although the release was slower than coated capsules, the release profiles were similar. On the other hand, release from the described formulations was faster than soft capsules and chewable tablets.

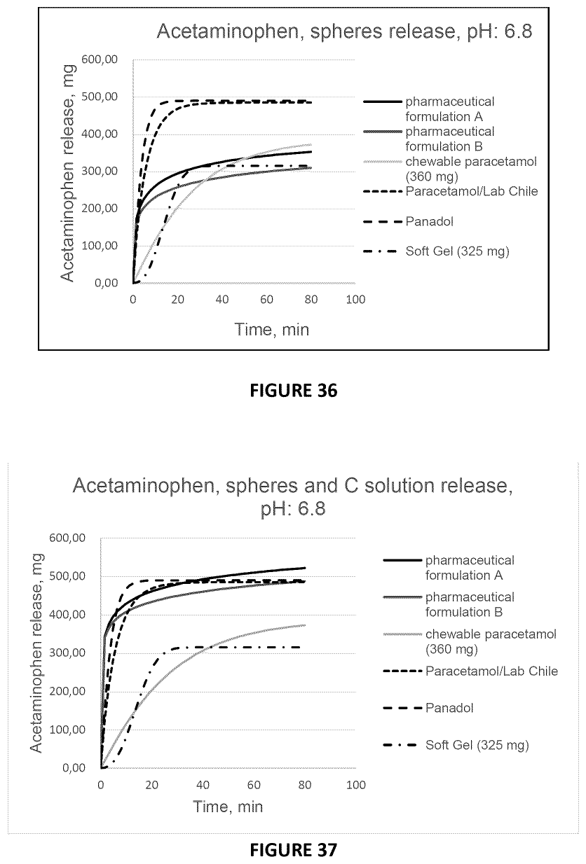

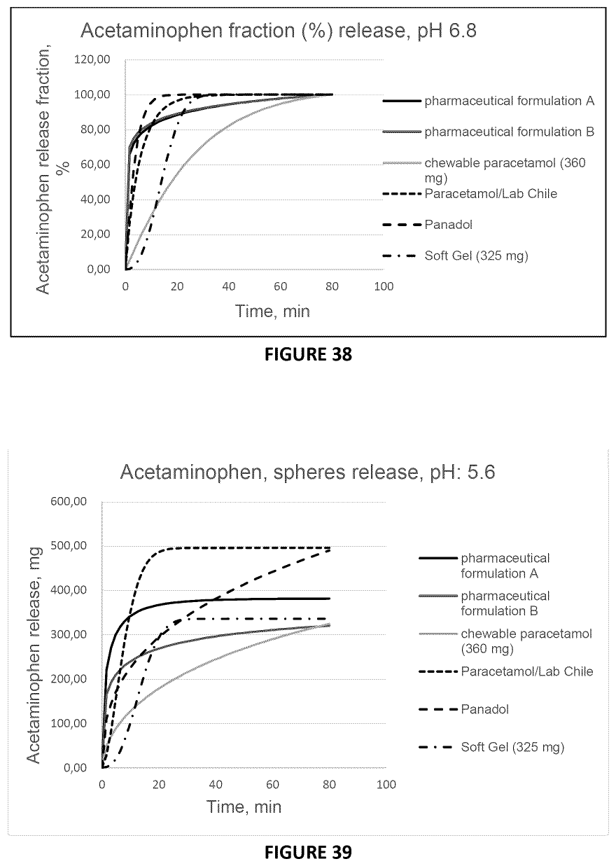

The release of acetaminophen at pH 1.2 surprisingly presents a similar behavior to another pHs media studied: simulated intestinal fluid (pH 6.8) and simulated duodenum fluid (pH 5.6) (FIG. 36 to FIG. 41). Once again, release was faster than soft capsules and chewable tablets. On the other hand, when release percentage of the described formulations was compared to commercial products, the described formulations presented a fraction release very similar to rapid-intermediate release product, FIGS. 38, 41 and 44. Furthermore, the Weibull maximum concentration in liquid (Fmax) and release rate of Higuchi models obtained to formulations proposed, had similar release values for soft capsules and it close to the tablets release containing gelatinized microparticles.

The above descriptions are only preferred embodiments of the present invention, and are not intended to limit the scope. Any modifications, equivalent replacements, improvements, made within the spirit and principles of the present invention shall be included in the protection of the present invention.

1. A single dose therapeutic beverage, comprising:
a beverage type liquid medium; and
a plurality of spherical- and/or spheroidal-shaped particles dispersed within the liquid medium;
wherein the plurality of the spherical- and/or spheroidal-shaped particles comprise at least one active pharmaceutical ingredient (API) disposed within the plurality of the spherical- and/or spheroidal-shaped particles;
wherein the API partially permeates into the beverage type liquid medium from within the plurality of the spherical- and/or spheroidal-shaped particles so that an amount of the API is partitioned between the plurality of the spherical- and/or spheroidal-shaped particles and the liquid medium containing the particles; and
wherein, upon administration to a subject, the therapeutic beverage is configured to provide an immediate dose of the API and a subsequent delayed release dose of the API.

2. The single dose therapeutic beverage of claim 1, wherein the percentage of APIs present in the liquid medium is from about 10% to about 50% w/v and the percentage of APIs present in the plurality of the spherical- and/or spheroidal-shaped particles is from about 50% to about 90% w/w; or
wherein the percentage of APIs present in the liquid medium is from about 15% to about 35% w/v and the percentage of APIs present in the plurality of the spherical- and/or spheroidal-shaped particles is from about 65% to about 85% w/w or
wherein the percentage of APIs present in the liquid medium is about 30% w/v and the percentage of APIs present in the plurality of the spherical- and/or spheroidal-shaped particles is about 70% w/w.

3. The single dose therapeutic beverage of claim 1, wherein the API is selected from the group consisting of analgesics, nonsteroidal anti-inflammatory drugs (NSAIDs), Cannabis derivatives, antiallergics, expectorants, antitussives, antibiotics, diuretics, hypotensives, hypoglycemics, antispasmodics, antidepressants, anti-ulcer drugs, vitamins, and supplements.

4. The single dose therapeutic beverage of claim 1, wherein the plurality of the spherical- and/or spheroidal-shaped particles further comprises at least one of:
one or more gelling agents selected from the group consisting of gellan gum, methyl cellulose, methyl ethyl cellulose, hydroxy propyl methyl cellulose, locust bean gum, carrageenan, sodium alginate, xanthan gum, gelatine, chitosan, derivatives of chitosan, and/or mixtures thereof; and
one or more agents for spherification selected from the group consisting of calcium chloride, calcium acetate, calcium citrate, calcium phosphate, monobasic calcium phosphate, and calcium gluconolactate.

5. The single dose therapeutic beverage of claim 4, further comprising one or more emulsifying agents selected from the group consisting of lecithin, fatty acid diglycerides and fatty acid esters, and polysaccharides.

6. The single dose therapeutic beverage of claim 5, further comprising one or more oily dispersing agents selected from the group consisting of canola oil, olive oil, almond oil, chia oil, corn oil, marigold oil, coconut oil, soybean oil, mineral oils and the like.

7. The single dose therapeutic beverage of claim 1, further comprising excipients selected from the group consisting of stabilizers, preservatives, antioxidants, viscosifying agents, flavoring agents, sweeteners, colorings, and pH stabilizers.

8. The single dose therapeutic beverage of claim 1, wherein the plurality of the spherical- and/or spheroidal-shaped particles have a diameter in a range from about 100 μm to about 6000 μm; from about 100 μm to about 4000 μm, from about 1000 μm to about 3000 μm, or from about 2000 μm to about 3000 μm.

9. The single dose therapeutic beverage of claim 1, wherein the therapeutic beverage is a packaged beverage provided in a unit that contains between about 10-1000 mL of the beverage, or between about 10-500 mL of the therapeutic beverage.

10. The single dose therapeutic beverage of claim 1, wherein the therapeutic beverage is in a form of an instant beverage.

11. The single dose therapeutic beverage of claim 1, wherein the beverage is for daily consumption once or more times a day.

12. The single dose therapeutic beverage of claim 1, wherein the API comprises a first API and a second API, different from the first API, and the first API is disposed within a first set of the plurality of spherical- and/or spheroidal-shaped particles and the second API is disposed with a second set of the plurality of spherical- and/or spheroidal-shaped particles.

13. A therapeutic beverage, comprising:
a beverage type liquid medium; and
a plurality of spherical- and/or spheroidal-shaped particles dispersed within the liquid medium,
wherein the plurality of the spherical- and/or spheroidal-shaped particles comprise at least one active pharmaceutical ingredient (API) disposed within the plurality of the spherical- and/or spheroidal-shaped particles;
wherein the spherical- and/or spheroidal-shaped particles have a diameter in a range from about 100 μm to about 6000 μm; and
wherein the API partially permeates into the beverage like liquid medium from within the plurality of the spherical- and/or spheroidal-shaped particles so that an amount of the API is partitioned between the plurality of the spherical- and/or spheroidal-shaped particles and the beverage like liquid medium containing the particles; and
wherein, upon administration to a subject, the therapeutic beverage is configured to provide an immediate dose of the API and a subsequent delayed release dose of the API.

14. The therapeutic beverage of claim 13, wherein the therapeutic beverage is a single dose therapeutic beverage disposed in a beverage container.

15. The therapeutic beverage of claim 13, wherein the percentage of APIs present in the liquid medium is from about 10% to about 50% w/v and the percentage of APIs present in the plurality of the spherical- and/or spheroidal-shaped particles is from about 50% to about 90% w/w; or the percentage of APIs present in the liquid medium is from about is from about 15% to about 35% w/v and the percentage of APIs present in the plurality of the spherical- and/or spheroidal-shaped particles is from about 65% to about 85% w/w; or the percentage of APIs present in the liquid medium is about 30% w/v and the percentage of APIs present in the spherical- and/or spheroidal-shaped particles is about 70% w/w.

16. The therapeutic beverage of claim 13, wherein the API comprises a first API and a second API, different from the first API, and the first API is disposed within a first set of the plurality of spherical- and/or spheroidal-shaped particles and the second API is disposed with a second set of the plurality of spherical- and/or spheroidal-shaped particles.

17. The therapeutic beverage of claim 13, wherein the API is selected from the group consisting of analgesics, nonsteroidal anti-inflammatory drugs (NSAIDs), Cannabis derivatives, antiallergics, expectorants, antitussives, antibiotics, diuretics, hypotensives, hypoglycemics, antispasmodics, antidepressants, anti-ulcer drugs, vitamins, and supplements.

18. The therapeutic beverage of claim 13, further comprising excipients selected from the group consisting of stabilizers, preservatives, antioxidants, viscosifying agents, flavoring agents, sweeteners, colorings, and pH stabilizers.

19. A single dose therapeutic beverage, comprising:
a beverage container;
a beverage type liquid medium disposed within the container;
a plurality of spherical- and/or spheroidal-shaped particles dispersed within the liquid medium;
wherein the plurality of the spherical- and/or spheroidal-shaped particles comprise at least one pharmaceutically active ingredient (API) disposed within the plurality of the spherical- and/or spheroidal-shaped particles;
wherein the spherical- and/or spheroidal-shaped particles have a diameter in a range from about 1000 μm to about 3000 μm;
wherein the beverage container contains between about 10-1000 mL of the single dose therapeutic beverage or between about 10-500 mL of the therapeutic beverage;
wherein the API partially permeates into the beverage like liquid medium from within the spherical- and/or spheroidal-shaped particles so that an amount of the API is partitioned between the spherical- and/or spheroidal-shaped particles and the beverage like liquid medium containing the particles; and
wherein, upon administration to a subject, the therapeutic beverage is configured to provide an immediate dose of the API and a subsequent delayed release dose of the API.

20. The single dose therapeutic beverage of claim 19, wherein the API comprises a first API and a second API, different from the first API, and the first API is disposed within a first set of the plurality of spherical- and/or spheroidal-shaped particles and the second API is disposed with a second set of the plurality of spherical- and/or spheroidal-shaped particles.

21. The single dose therapeutic beverage of claim 19, wherein the percentage of APIs present in the liquid medium is from about 10% to about 50% w/v and the percentage of APIs present in the plurality of the spherical- and/or spheroidal-shaped particles is from about 50% to about 90% w/w; or the percentage of APIs present in the liquid medium is from about is from about 15% to about 35% w/v and the percentage of APIs present in the plurality of the spherical- and/or spheroidal-shaped particles is from about 65% to about 85% w/w; or the percentage of APIs present in the liquid medium is about 30% w/v and the percentage of APIs present in the spherical- and/or spheroidal-shaped particles is about 70% w/w.

22. The single dose therapeutic beverage of claim 19, wherein the therapeutic beverage is in a form of an instant beverage.

23. The therapeutic beverage of claim 19, wherein the API is selected from the group consisting of analgesics, nonsteroidal anti-inflammatory drugs (NSAIDs), Cannabis derivatives, antiallergics expectorants, antitussives, antibiotics, diuretics, hypotensives, hypoglycemics, antispasmodics, antidepressants, anti-ulcer drugs, vitamins, and supplements.

24. The therapeutic beverage of claim 19, further comprising excipients selected from the group consisting of stabilizers, preservatives, antioxidants, viscosifying agents, flavoring agents, sweeteners, colorings, and pH stabilizers.

25. A single dose therapeutic beverage, comprising:
a beverage container;
a beverage type liquid medium disposed within the container;
a plurality of spherical- and/or spheroidal-shaped particles dispersed within the liquid medium;
wherein the spherical- and/or spheroidal-shaped particles comprise at least one pharmaceutically active ingredient (API) disposed within the spherical- and/or spheroidal-shaped particles;
wherein the spherical- and/or spheroidal-shaped particles have a diameter in a range from about 100 μm to about 6000 μm;
wherein the beverage container contains between 10-1000 mL of the therapeutic beverage;
wherein the API partially permeates into the beverage like liquid medium from within the spherical- and/or spheroidal-shaped particles so that an amount of the API is partitioned between the spherical- and/or spheroidal-shaped particles and the beverage like liquid medium containing the particles;
wherein, upon administration to a subject, the therapeutic beverage is configured to provide an immediate dose of the API and a subsequent delayed release dose of the API; and
wherein the percentage of APIs present in the liquid medium is from about 10% to about 50% w/v and the percentage of APIs present in the plurality of the spherical- and/or spheroidal-shaped particles is from about 50% to about 90% w/w.

26. The single dose therapeutic beverage of claim 25, wherein the API comprises a first API and a second API, different from the first API, and the first API is disposed within a first set of the plurality of spherical- and/or spheroidal-shaped particles and the second API is disposed with a second set of the plurality of spherical- and/or spheroidal-shaped particles.

27. The single dose therapeutic beverage of claim 25, wherein the percentage of APIs present in the liquid medium is about 30% w/v and the percentage of APIs present in the spherical- and/or spheroidal-shaped particles is about 70% w/w.

28. The therapeutic beverage of claim 25, wherein the API is selected from the group consisting of analgesics, nonsteroidal anti-inflammatory drugs (NSAIDs), Cannabis derivatives, antiallergics expectorants, antitussives, antibiotics, diuretics, hypotensives, hypoglycemics, antispasmodics, antidepressants, anti-ulcer drugs, vitamins, and supplements.

29. A single dose therapeutic beverage, comprising:
a beverage container;
a beverage type liquid medium disposed within the container;
a plurality of spherical- and/or spheroidal-shaped particles dispersed within the liquid medium;
wherein the plurality of the spherical- and/or spheroidal-shaped particles comprise at least one active pharmaceutical ingredient (API) disposed within the plurality of the spherical- and/or spheroidal-shaped particles;
wherein the spherical- and/or spheroidal-shaped particles have a diameter in a range from about 100 μm to about 6000 μm;
wherein the beverage container contains between about 10-1000 mL of the single dose therapeutic beverage, or between about 10-500 mL of the single dose therapeutic beverage; and
wherein the percentage of APIs present in the liquid medium is from about 10% to about 50% w/v and the percentage of APIs present in the plurality of the spherical- and/or spheroidal-shaped particles is from about 50% to about 90% w/w; or the percentage of APIs present in the liquid medium is from about is from about 15% to about 35% w/v and the percentage of APIs present in the plurality of the spherical- and/or spheroidal-shaped particles is from about 65% to about 85% w/w; or the percentage of APIs present in the liquid medium is about 30% w/v and the percentage of APIs present in the plurality of the spherical- and/or spheroidal-shaped particles is about 70% w/w.

30. The single dose therapeutic beverage of claim 29, wherein the plurality of the spherical- and/or spheroidal-shaped particles have a diameter in a range from about 100 μm to about 4000 μm, from about 1000 μm to about 3000 μm, or from about 2000 μm to about 3000 μm.

31. The single dose therapeutic beverage of claim 29, wherein the API is selected from the group consisting of analgesics, nonsteroidal anti-inflammatory drugs (NSAIDs), Cannabis derivatives, antiallergics, expectorants, antitussives, antibiotics, diuretics, hypotensives, hypoglycemics, antispasmodics, antidepressants, anti-ulcer drugs, vitamins, and supplements.

32. The single dose therapeutic beverage of claim 29, wherein the plurality of the spherical- and/or spheroidal-shaped particles further comprises at least one of:
one or more gelling agents selected from the group consisting of gellan gum, methyl cellulose, methyl ethyl cellulose, hydroxy propyl methyl cellulose, locust bean gum, carrageenan, sodium alginate, xanthan gum, gelatine, chitosan, derivatives of chitosan, and/or mixtures thereof; and
one or more agents for spherification selected from the group consisting of calcium chloride, calcium acetate, calcium citrate, calcium phosphate, monobasic calcium phosphate, and calcium gluconolactate.

33. The single dose therapeutic beverage of claim 32, further comprising one or more emulsifying agents selected from the group consisting of lecithin, fatty acid diglycerides and fatty acid esters, and polysaccharides.

34. The single dose therapeutic beverage of claim 33, further comprising one or more oily dispersing agents selected from the group consisting of canola oil, olive oil, almond oil, chia oil, corn oil, marigold oil, coconut oil, soybean oil, mineral oils and the like.

35. The single dose therapeutic beverage of claim 29, further comprising excipients selected from the group consisting of stabilizers, preservatives, antioxidants, viscosifying agents, flavoring agents, sweeteners, colorings, and pH stabilizers.

36. The single dose therapeutic beverage of claim 29, wherein the therapeutic beverage is in a form of an instant beverage.

37. The single dose therapeutic beverage of claim 29, wherein the beverage is for daily consumption once or more times a day.

38. The single dose therapeutic beverage of claim 29, wherein the API comprises a first API and a second API, different from the first API, and the first API is disposed within a first set of the plurality of spherical- and/or spheroidal-shaped particles and the second API is disposed with a second set of the plurality of spherical- and/or spheroidal-shaped particles.

39. A method of preparing a single dose therapeutic beverage, comprising:
i) suspending at least one active pharmaceutical ingredient (API) in an aqueous medium and/or in an oily medium;
ii) adding at least one gelling agent to step i) while stirring to produce a mixture;
iii) dropping the mixture obtained in step ii) onto a spherification agent while stirring to produce spherical- and/or spheroidal-shaped particles comprising the at least one API;
iv) removing by filtration the spherification agent to separate spherical- and/or spheroidal-shaped particles comprising the at least one API;
v) washing the separated spherical- and/or spheroidal-shaped particles comprising the at least one API with demineralized and/or deionized water; and
vi) suspending the washed spherical- and/or spheroidal-shaped particles comprising the at least one API in the liquid medium; and
vii. introducing the single dose therapeutic beverage into a beverage container;
wherein the API partially permeates into the beverage like liquid medium from within the spherical- and/or spheroidal-shaped particles so that an amount of the API is partitioned between the spherical- and/or spheroidal-shaped particles and the beverage like liquid medium containing the particles;
wherein, upon administration to a subject, the therapeutic beverage is configured to provide an immediate dose of the API and a subsequent delayed release dose of the API; and
wherein the percentage of APIs present in the liquid medium is from about 10% to about 50% w/v and the percentage of APIs present in the plurality of the spherical- and/or spheroidal-shaped particles is from about 50% to about 90% w/w.

40. The method of claim 39, wherein in step iii) the spherification agent can contain at least one gelling agent.

41. The method of claim 39, wherein in step ii) the temperature is from about 25° C. to about 60° C.

42. The method of claim 39, wherein in step iii) the spherification agent is CaCl₂ solution in the range from about 1.5% to about 6% w/v.

43. A method of preparing a single dose therapeutic beverage, comprising:
i) suspending at least one active pharmaceutical ingredient (API) in an alkaline medium and/or in an oily medium;
ii) adding a gelling agent to step i) while stirring to produce a mixture;
iii) dropping the mixture obtained in step ii) onto another gelling agent while stirring to produce spherical- and/or spheroidal-shaped particles comprising the at least one API;
iv) removing by filtration the spherification agent to separate spherical- and/or spheroidal-shaped particles comprising the at least one API;
v) washing the separated spherical- and/or spheroidal-shaped particles comprising the at least one API with demineralized and/or deionized water; and
vi) suspending the washed spherical- and/or spheroidal-shaped particles comprising the at least one API in the liquid medium; and
vii. introducing the single dose therapeutic beverage into a beverage container;
wherein the API partially permeates into the beverage like liquid medium from within the spherical- and/or spheroidal-shaped particles so that an amount of the API is partitioned between the spherical- and/or spheroidal-shaped particles and the beverage like liquid medium containing the particles;
wherein, upon administration to a subject, the therapeutic beverage is configured to provide an immediate dose of the API and a subsequent delayed release dose of the API; and
wherein the percentage of APIs present in the liquid medium is from about 10% to about 50% w/v and the percentage of APIs present in the plurality of the spherical- and/or spheroidal-shaped particles is from about 50% to about 90% w/w.

44. The method of claim 43, wherein in step ii) the temperature is from about 25° C. to about 60° C.

45. The method of claim 43, wherein in step iii) the spherification agent is CaCl₂ solution in the range from about 1.5% to about 6% w/v.
